(19) United States
(12) Patent Application Publication
(10) Pub. No.: US 2024/0024067 A1
(43) Pub. Date: Jan. 25, 2024

(54) MEDICAL DEVICE SUPPORT AND METHOD OF USE

(71) Applicant: EDWARDS LIFESCIENCES CORPORATION, Irvine, CA (US)

(72) Inventors: Robert Bowes, Trabuco Canyon, CA (US); Sergio Delgado, Irvine, CA (US); Ismail Thazhapilavil, Irvine, CA (US); Mindy Lee Ann Black, Huntington Beach, CA (US); Waina Michelle Chu, Tustin, CA (US); Michael J. Popp, Irvine, CA (US); Hsingching C. Hsu, Santa Ana, CA (US); Raffi Sempad Pinedjian, Fountain Valley, CA (US); Scot Herbst, Santa Cruz, CA (US); Jeffrey William Servaites, Oakland, CA (US); Carl Hermann James Poppe, Sebastopol, CA (US)

(21) Appl. No.: 18/476,857

(22) Filed: Sep. 28, 2023

Related U.S. Application Data
(63) Continuation of application No. PCT/US2022/021758, filed on Mar. 24, 2022.
(60) Provisional application No. 63/301,989, filed on Jan. 21, 2022.
(60) Provisional application No. 63/168,204, filed on Mar. 30, 2021.

(51) Int. Cl. A61B 90/57

(52) U.S. Cl. CPC A61B 90/57; A61B 2090/571; A61M 25/0113

(57) ABSTRACT

A stabilizing base includes one or more legs and a platform attached to the one or more legs. One or more of a height of the platform relative to an operating table, a position of the platform along a width of the operating table, a position of the platform along a length of the operating table, and an angular position of the platform relative to the operating table can be adjusted.

### RELATED APPLICATIONS

The present application is a continuation of Patent Cooperation Treaty Application No. PCT/US2022/021758, filed on Mar. 24, 2022, which claims the benefit of U.S. Provisional Patent Application No. 63/301,989, filed on Jan. 21, 2022, titled “Medical Device Support and Method of Use” and U.S. Provisional Patent Application No. 63/168,204, filed on Mar. 30, 2021, which are both incorporated herein by reference in their entirety.

### BACKGROUND

Endovascular delivery systems can be used in various procedures to deliver medical devices or instruments to a target location inside a patient's body that are not readily accessible by surgery or where access without surgery is desirable. The systems described herein can be used to deliver medical devices (stents, heart valve, grafts, clips, rings, repair devices, valve treatment devices, etc.) to a location in a patient's body.

Access to a target location inside the patient's body can be achieved by inserting and guiding the delivery system through a pathway or lumen in the body, including, but not limited to, a blood vessel, an esophagus, a trachea, any portion of the gastrointestinal tract, a lymphatic vessel, to name a few. Catheters are known in the art and have been commonly used to reach target locations inside a patient's body.

In some procedures, one or more catheters can be used to deliver a device for repairing or replacing a native heart valve. The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant implantable devices or implants (e.g., an implantable prosthetic device, a prosthetic spacer device, a valve repair device, a valve replacement device, etc.) in a manner that is much less invasive than open heart surgery. As one example, a transvascular technique useable for accessing the native mitral and aortic valves is a trans-septal technique. Trans-septal techniques can comprise advancing a catheter into the right atrium (e.g., inserting a catheter into the right femoral vein, up the inferior vena cava and into the right atrium). The septum can then be punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Stabilizing systems and/or apparatuses can be used to support and/or hold one or more components of a delivery system, e.g., a catheter assembly, at a stable position. Systems can include one or more stabilizing systems/apparatuses and a delivery system/catheter assembly. A valve repair device, a replacement valve, an annuloplasty ring or another implantable device can be coupled to the delivery system/catheter assembly.

An example stabilizing systems and/or apparatus for a medical system includes a base, a post, a rail, and an adjustment assembly. The base can be placed on one or more surfaces, such as a table. The post is attached to the base and supports the rail. The adjustment assembly can allow the post and/or the rail to move relative to the base. The rail is moveably attached to the post and is configured to receive the medical system.

An example stabilizing apparatus for a medical system includes a frame, at least one base, a rail, and an adjustment assembly. The bases can be placed on one or more surfaces, such as a table. The frame is attached to the bases and supports the rail. The adjustment assembly can allow the frame and/or the rail to move relative to the base. The rail is moveably attached to the frame and is configured to receive the medical system.

An example stabilizing apparatus for a medical system includes a mount, an arm, a rail, and an adjustment assembly. The mount can be attached to one or more surfaces, such as a table. The arm is attached to the mount and supports the rail. The adjustment assembly can allow the arm and/or the rail to move relative to the mount. The rail is moveably attached to the arm and is configured to receive the medical system.

An example stabilizing apparatus for a medical system includes a stabilizing base configured as a mat having at least one channel. The mat can be placed directly on a patient's body. The channel is configured to receive the medical system.

In some implementations, an example system for supporting a medical device includes a stabilizing base that comprises a plurality of legs and a platform attached to the plurality of legs. The system also includes a stabilizing system for receiving the medical device, wherein the stabilization system is removably attachable to the platform.

In some implementations, one or more of the plurality of legs is adjustable to change a vertical, horizontal, and/or angular position of the platform.

In some implementations, the stabilizing system is connected to the platform via metal plates.

In some implementations, one or more of the plurality of legs comprise an outer portion and an inner portion movably disposed within said outer portion.

In some implementations, one or more of the plurality of legs comprise a gas spring mechanism to adjust the vertical position of the inner portion relative to the outer portion.

In some implementations, the stabilizing base includes a first plate and a second plate, wherein the first plate and the second plate are parallel to one another and extend vertically downward from the platform on either side of the platform.

In some implementations, a knob mechanism extends through at least one of the first plate and the second plate.

In some implementations, the system includes a mechanism to control the length of one or more of the plurality of legs. In some implementations, the mechanism is an electromechanical mechanism. In some implementations, the mechanism is a mechanical mechanism. In some implementations, the mechanism is a motorized mechanism. In some implementations, the mechanism is a hydraulic mechanism.

In some implementations, the stabilizing system is a rail system for receiving the medical device, wherein the rail system is removably attachable to the platform.

In some implementations, the medical device is a delivery system couplable to the stabilizing system and the overall system includes the delivery system;

In some implementations, the system includes at least one retaining member for retaining each of the plurality of support legs in the stowed position;

In some implementations, the retaining member is attached to a bottom cover of the platform;

In some implementations, the retaining member comprises an elastomeric pad.

In some implementations, the system includes a latch mechanism for prohibiting the rotation of the first pair of legs and the second pair of legs when the first pair of legs and the second pair of legs are in the stowed position and the deployed position.

In some implementations, the system the latch mechanism includes rotating catch members and sliding latch members. The rotating catch members extend between the support legs of the first pair of legs and the support legs of the second pair of legs. The sliding latch members engage the rotating catch members to prohibit rotation of the rotating catch members.

In some implementations, the system includes a tightening screw that engages extension members of the rotating catch members.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain implementations will be made by reference to various aspects of the appended drawings. It is appreciated that these drawings depict only typical implementations of the present disclosure and are therefore not to be considered limiting of the scope of the disclosure. Moreover, while the figures may be drawn to scale for some implementations, the figures are not necessarily drawn to scale for all implementations. Implementations and other features and advantages of the present disclosure will be described and explained with additional specificity and detail through the use of the accompanying drawings in which:

FIG. 1 shows an operating table having thereon an example stabilizing base for supporting a medical device/system (e.g., delivery system) useable to implant an implantable device;

FIGS. 2-3 show an example stabilizing base for supporting a medical device/system (e.g., delivery system);

FIGS. 4-7 show various views of an example stabilizing base for supporting a medical device/system (e.g., delivery system);

FIG. 8 shows an example stabilizing base for supporting a medical device/system (e.g., delivery system);

FIGS. 9-12 show various views of an example stabilizing base for supporting a medical device/system (e.g., delivery system);

FIGS. 13-16 show various views of an example stabilizing base for supporting a medical device/system (e.g., delivery system);

FIGS. 17-20 show various views of an example stabilizing base for supporting a medical device/system (e.g., delivery system);

FIGS. 21-25 show various views of an example stabilizing base for supporting a medical device/system (e.g., delivery system);

FIG. 26 shows an example stabilizing base for supporting a medical device/system (e.g., delivery system);

FIGS. 27-30 show various views of an example stabilizing base for supporting a medical device/system (e.g., delivery system);

FIGS. 31-34 show various views of an example stabilizing base for supporting a medical device/system (e.g., delivery system);

FIGS. 35-36 show various views of an example stabilizing base for supporting a medical device/system (e.g., delivery system);

FIGS. 37-40 show various views of an example stabilizing base for supporting a medical device/system (e.g., delivery system);

FIGS. 41-44 show various views of an example stabilizing base for supporting a medical device/system (e.g., delivery system);

FIGS. 45-48 show various views of an example stabilizing base for supporting a medical device/system (e.g., delivery system);

FIGS. 49-51 show various views of an example stabilizing base for supporting a medical device/system (e.g., delivery system);

FIGS. 52-54 show various views of an example stabilizing base for supporting a medical device/system (e.g., delivery system);

FIG. 55 shows an example stabilizing base for supporting a medical device/system (e.g., delivery system);

FIGS. 56-66 show various views of an example stabilizing base for supporting a medical device/system (e.g., delivery system);

FIGS. 67-72 show various views of an example stabilizing base for supporting a medical device/system (e.g., delivery system);

FIGS. 73-74 show various views of an example stabilizing base for supporting a medical device/system (e.g., delivery system);

FIGS. 75-84 show various views of an example stabilizing base for supporting a medical device/system (e.g., delivery system) and components thereof;

FIGS. 85-87 show various views of an example stabilizing base for supporting a medical device/system (e.g., delivery system);

FIG. 88 shows a perspective view of an example stabilizing base for supporting a medical device/system (e.g., delivery system);

FIGS. 89-90 show various views of an example stabilizing base for supporting a medical device/system (e.g., delivery system);

FIGS. 91-94 show various views of an example stabilizing base for supporting a medical device/system (e.g., delivery system);

FIGS. 95-104 show various views of an example stabilizing base for supporting a medical device/system (e.g., delivery system); and

FIGS. 105-118 show various views of an example stabilizing base for supporting a medical device/system (e.g., delivery system).

### DETAILED DESCRIPTION

The following description refers to the accompanying drawings, which illustrate specific implementations of the present disclosure. Other implementations having different structures and operation do not depart from the scope of the present disclosure.

Example implementations of the present disclosure are directed to devices and systems for stabilizing medical devices and systems. It should be noted that various implementations of stabilization devices are disclosed herein, and any combination of the features of these implementations can be made unless specifically excluded. In other words, individual components of the disclosed devices and systems can be combined unless mutually exclusive or otherwise physically impossible.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

During surgical procedures using one or more catheters, it typically is beneficial for the operator to be able to precisely control the operation of the catheter because the catheter must be directed through a patient's vasculature. This includes mechanisms that allow the catheter to be bent to assist in navigating the vasculature, and mechanisms that control deployment of the implantable device. During a procedure, the operator can control the catheter using a handle, which can provide controls for extending, retracting, and bending the catheter, including during navigating the patient's vasculature to the delivery or repair site.

Transcatheter procedures may have a long duration, and it may be inconvenient for an operator to manually maintain the position of the catheter handle during the entire procedure. While it may be desirable to adjust the location of the catheter handle relative to the patient at some points during the procedure, at other times it can be desirable to maintain the position of the catheter handle relative to the patient, such as to maintain the depth of insertion of the catheter or the rotational position of the handle.

Stabilizing systems/devices and stabilizing bases for supporting the same can be used to hold a medical device or medical system-such as a delivery system (e.g., delivery catheter, series of catheters, catheter assembly, handles, etc.) for implanting an implantable device-relative to a patient during a surgical procedure. The stabilizing base can also be used as a work surface to hold other tools, implements, or materials, or the like that are needed for the particular procedure. Example stabilizing systems/devices support and position the medical device/system in a desired location so that the medical device/system does not move without the direction of the operator of the device. The stabilizing system/sdevices disclosed herein can also be easily adjusted so that when the operator does want to reconfigure or move the medical system/device (e.g., delivery system), such movements are easily accomplished.

The example stabilizing systems/devices and stabilizing bases disclosed herein can also accommodate a sterile barrier, such as a drape, arranged between the stabilizing system/device and the stabilizing base. The stabilizing system/device can remain adjustable relative to the base without removing the sterile barrier. This arrangement provides significant advantages over prior art methods of fixing stabilizing systems/devices to stabilizing bases using clamps that must be opened and moved to adjust the position of the stabilizing system/device relative to the stabilizing base. To accommodate medical device/systems of different lengths, the example stabilizing systems/devices disclosed herein can span multiple example stabilizing bases, thereby enabling an operator to configure work surfaces of different lengths using the same components.

The stabilizing systems/devices herein can comprise one or more rail systems. The one or more rail systems can be configured to be assembled to a stabilizing base with a sterile barrier provided between the two. A platform of the stabilizing system/device can engage the rail system to securely attach the rail system to the stabilizing base. Various mechanisms can be employed to connect one or more rail systems and the stabilizing base to facilitate adjustment of the relative positions of these components, and the locking or release thereof. The stabilizing methods for various implementations can be different and are more fully discussed below with respect to each implementation. Additional information regarding these as well as various other types stabilization systems, devices, methods, etc. that the inventions herein can be used with can be found in U.S. Provisional Application No. 63/073,392, U.S. patent application Ser. No. 15/951,830, U.S. patent application Ser. No. 15/905,257, U.S. patent application Ser. No. 16/582,307, U.S. patent application Ser. No. 17/066,416, and PCT Application No. PCT/US2021/048333, each of which is incorporated herein by reference in its entirety for all purposes.

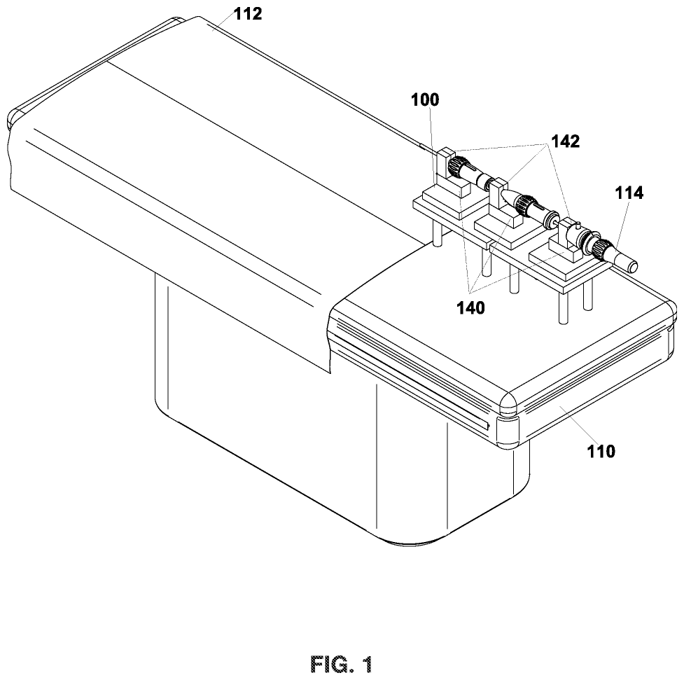

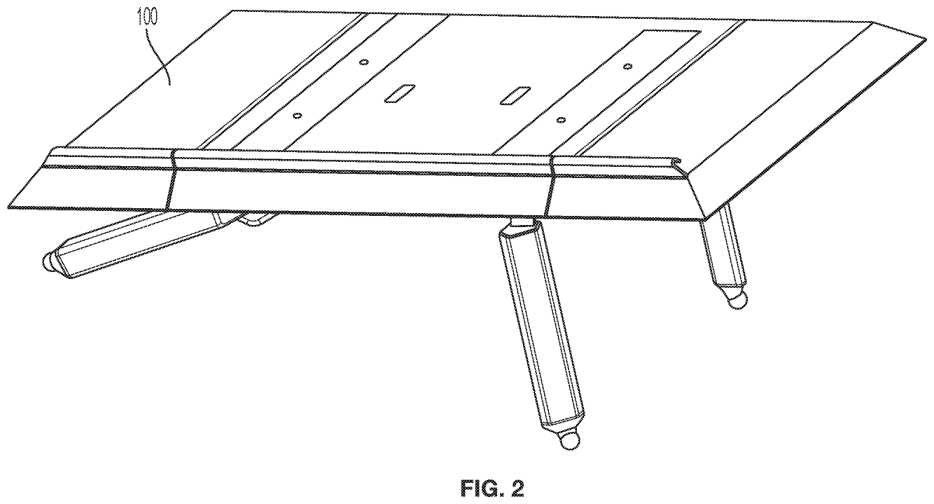

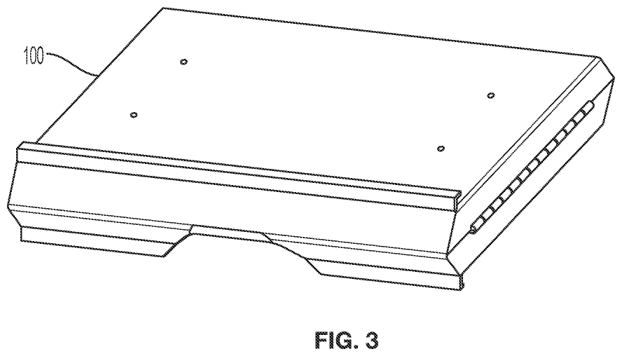

FIG. 1 shows a table, e.g., an operating table, having thereon an example stabilizing base for supporting a medical device/medical system (e.g., a delivery device, delivery system, one or more catheters, one or more handles, a catheter assembly, one or more devices, etc.) useable to implant an implantable device. A patient can also be positioned on the operating table, with the stabilizing base positioned near the patient, e.g., to the side of the patient, between the patient's legs, on a portion of the patient, etc. FIGS. 2-3 also show an example stabilizing base 100 which can be the same as or similar to the example stabilizing base 100 in FIG. 1 or can have one or more differences therefrom.

The stabilizing base 100 of any of FIGS. 1-3 can incorporate any of the features of stabilizing bases disclosed herein and can be made from any suitable material, such as, for example, metal or plastic. The stabilizing base 100 has a base, such as legs, posts or a frame, for elevating and supporting a platform above a patient undergoing a procedure with a medical device/system, such as a catheter assembly 114, that is stabilized by the stabilizing base 100. The base can include feet (not shown) to further stabilize the legs and/or so that fewer than four legs are needed to support the stabilizing base 100 in a stable base condition. The stabilizing base 100 can also include a variety of base attachment locations for reconfiguring the stabilizing method, for example, to avoid obstacles. In some implementations, the legs are movable to different angles. In some implementations, the legs are extendable or retractable to different lengths to change the angle of the platform. In some implementations, the legs can be set at fixed angles, while the legs can still be extended or retracted to different lengths to change the angle of the platform.

In some implementations, a stabilizing base comprises a plurality of legs and a platform attached to the plurality of legs. In some implementations a stabilizing system for receiving a medical device is removably attachable to the platform. In some implementations, the stabilizing system is connected to the platform via metal plates.

In some implementations, one or more of the plurality of legs is adjustable to change a vertical, horizontal, and/or angular position of the platform. In some implementations, the length of one or more legs is adjustable. In some implementations, an angle of one or more legs is adjustable.

In some implementations, the system includes a mechanism to control the length of one or more of the plurality of legs. In some implementations, the mechanism is an electromechanical mechanism. In some implementations, the mechanism is a mechanical mechanism. In some implementations, the mechanism is a motorized mechanism. In some implementations, the mechanism is a hydraulic mechanism.

In some implementations, one or more of the plurality of legs comprise an outer portion and an inner portion movably disposed within said outer portion. In some implementations, the inner portion and the outer portion can telescope or otherwise move relative to each other to change a length of a leg. In some implementations, one or more of the plurality of legs comprise a mechanism (e.g., a hydraulic mechanism, an electromechanical mechanism, a motorized mechanism, a gas spring mechanism, a mechanical mechanism, etc.) to adjust the vertical position of the inner portion relative to the outer portion.

In some implementations, the stabilizing base includes a first plate and a second plate, wherein the first plate and the second plate are parallel to one another and extend vertically downward from the platform on either side of the platform. In some implementations, a knob mechanism extends through at least one of the first plate and the second plate.

In some implementations, the stabilizing system is a rail system for receiving the medical device, wherein the rail system is removably attachable to the platform.

In some implementations, the stabilizing system comprises a rail system 140. In some implementations, the rail system is moveably—and optionally, removably—attached to the platform of the stabilizing base 100 so that the delivery system/catheter assembly 114 can be moved along with the rail system relative to the stabilizing base 100. Example rail systems that can be used with the stabilizing bases of the present application are disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties for all purposes.

The stabilizing base 100 can rest on a support surface, such as a table 110, e.g. an operating table. Alternatively or additionally, the stabilizing base 100 can be attached to the table 110 such as by mounting the stabilizing base 100 to bed rails on the side of the table 110 or another component of the table. Clamps 142 can be attached to the rail system 140 to secure the medical device/system, such as the catheter assembly 114, to the rail system 240. Example clamps that can be used with the stabilizing bases of the present application are disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties.

A sterile barrier 112, such as a drape, can be placed on top of the table 110, either below the stabilizing base 100 or in between portions of the stabilizing base 100. The illustrated barrier 112 can be moved between the stabilizing base 100 and the rail system 140 or an additional barrier can be placed between the stabilizing base 100 and the rail system 140. The rail system 140 can be configured to be entirely or partially retained by features of the stabilizing base 100 so that when the stabilizing base is covered by a sterile barrier 112, such as a drape, the rail system 140 can still be attached to the stabilizing base 100. That is, the rail system 140 can be attached to the stabilizing base 100 without attachment means, such as fasteners, that would pierce or puncture the sterile barrier arranged between the stabilizing base 100 and the rail system 140. In some implementations, the rail system 140 can be attached to the stabilizing base 100 using magnetic plates.

The stabilizing base 100 can take a wide variety of different forms. In the example illustrated by FIG. 1, the stabilizing base 100 can comprise one or more (two in the illustrated implementation) tables or bases that each have four legs. In some implementations, the stabilizing base 100 includes two or three adjustable legs. The legs can be adjusted to change the height and angle of the stabilizing base 100 relative to the operating table 110, e.g., by changing an angle of one or more legs, by changing a length of one or more legs, and/or a combination of these. As illustrated by FIG. 3, the table of FIG. 2 can be folded to a smaller configuration for shipping and/or storage. In some implementations, the rail system 140 and/or clamps 142 can be stored inside the folded stabilizing base.

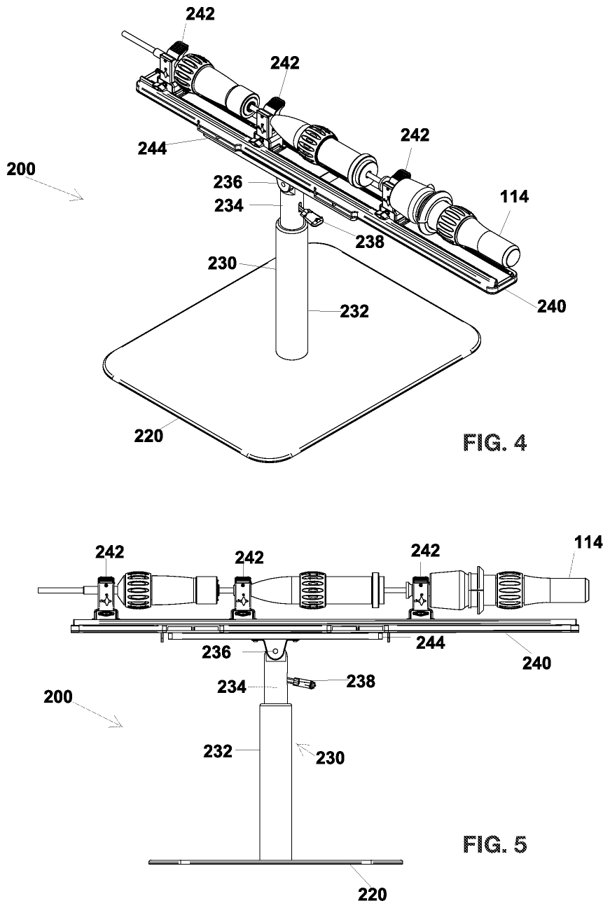

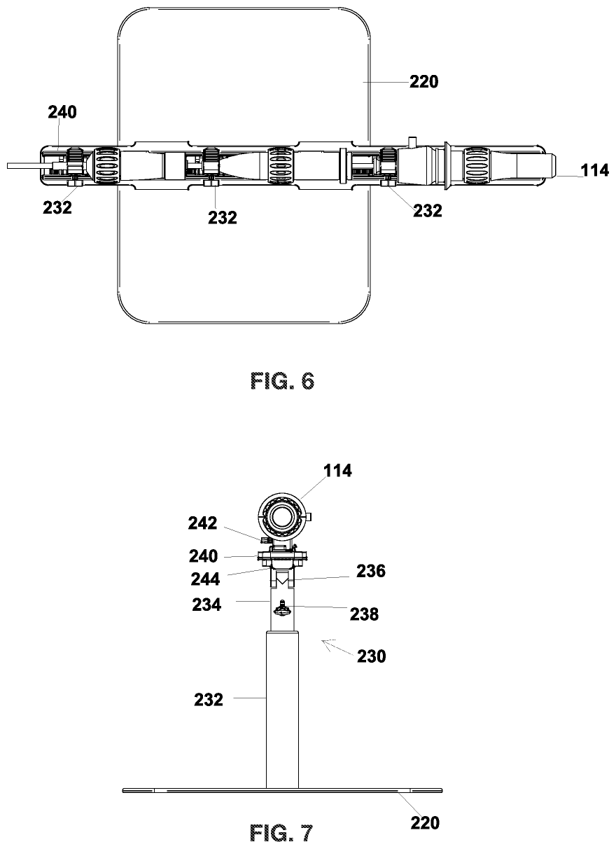

Referring now to FIGS. 4-7 an example stabilizing base 200 for supporting a medical device/system is shown. The stabilizing base 200 can incorporate any of the features of stabilizing bases disclosed herein and can be made from any suitable material, such as metal or plastic.

The stabilizing base 200 includes a base plate 220, a post 230, a platform 244, and a rail system 240. The post 230 extends vertically upwards from the base plate 220 and attaches to the platform 244. The platform 244 removably attaches to the rail system 240 (though in some implementations, the platform and rail system can be integrally formed). Clamps 242 can be attached to the rail system 240 to secure the medical device/system, such as a delivery system or catheter assembly 114, to the rail system 240. In some implementations, the rail system 240 and/or the clamps 242 are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 200 maintains stability by having a broad base plate 220 to rest on a surface such as a patient's table.

The base plate 220 can be a substantially flat plate of any suitable shape and dimension to provide counterbalance and support to the medical device/system secured to the stabilizing base 200. The base plate can be square, round, hexagonal, and the like. The base plate 420 can be solid or can have a void space within the center of the plate. In some implementations, the base plate 220 can be a solid rectangular plate.

The post 230 can be joined to the base plate 220 by any suitable means, such as welding, an adhesive, or the like. In some implementations, the post 230 can be movably attached to the base plate 220, such as by a hinge mechanism, to allow the post 230 to pivot relative to the base plate 220. The post 230 can be any suitable shape, such as square, circular, or oblong. In some implementations, the post 230 has an outer portion 232 and an inner portion 234, which is movably disposed within the outer portion 232. The vertical position of the inner portion 234 of the post 230 within the outer portion 232 of the post 230 can be adjusted via a gas spring mechanism (not shown) within the post 230. The gas spring mechanism can be locked in place using an actuator 238, such as a button, a tab, a switch, or the like. In some implementations, the gas spring mechanism is tuned to balance out the weight of the catheter assembly 114, such that the vertical position of the post 230 can be adjusted easily by a user.

The gas spring mechanism within the post 230 can be adjusted by unlocking the actuator 238 and moving the inner portion 234 of the post 230 upwards or downwards within the outer portion 232 of the post 230. The movement of the inner portion 234 within the outer portion 232 of the post 230 changes the distance between the base plate 220 and the platform 244. Adjusting the height of the platform 244 allows the rail system 240, and the catheter assembly 114 secured thereto, to be positioned at a desired height above a patient.

The platform 244 can be joined to the post 230 by any suitable means, such as welding, fasteners, and/or an adhesive, or the like. In some implementations, the inner portion 234 of the post 230 can connect to the platform 244 via an adjustable connection, such as a hinge 236, which allows the platform 244 to pivot relative to the post 230. In some implementations, the hinge 236 can be unlocked, to allow for the adjustment of the radial position of the platform 244 relative to the post 230, and the hinge 236 can be locked to prevent further movement of the platform 244 once the desired radial position has been established. In some implementations, the hinge 236 can be tuned or selected, such as by a spring or counterweight mechanism, to prevent radial movement of the platform 244 unless the hinge 236 is acted upon by applying a sufficient upwards or downwards force to the platform 244.

The rail system 240 can be removably attached to or combined with the platform 244 of the stabilizing base 200 by any suitable means, such as, for example, with fasteners, threaded fasteners, snaps, clamps, latches, friction fit, spring-loaded clamps, hook and loop fasteners, magnets, or the like. The rail system 240 can be configured to be entirely or partially retained by features of the platform 244 of the stabilizing base 200 so that when the stabilizing base is covered by a sterile barrier (not shown), such as a drape, the rail system 240 can still be attached to the stabilizing base 200. The illustrated barriers 112 can be placed between the stabilizing base 200 and the rail system 240. The rail system 240 can be attached to the stabilizing base 200 without attachment means, such as fasteners, that would pierce or puncture the sterile barrier arranged between the platform 244 and the rail system 240. In some implementations, the rail system can be attached to the stabilizing base 200 using magnetic plates or snap connections that do not puncture a sterile barrier. In some implementations, the horizontal position of rail system 240 can be adjusted relative to the platform 244, such as by sliding the rail system 240 forwards and backwards in relation to the platform 244.

The base plate 220 can be placed flush against the surface of a table 110 (as shown in FIG. 1), and a patient's legs can rest atop the base plate 220, such that the post 230 extends vertically upwards in between the patient's legs. The position of the delivery system/catheter assembly 114 relative to the patient can be optimized by adjusting the positions of the various elements of the stabilizing base 200, such as by raising and lowering the height of the post 230, adjusting the pitch of the platform 244 relative to the post 230, and/or sliding the rail system 240 forward or backwards relative to the platform 244.

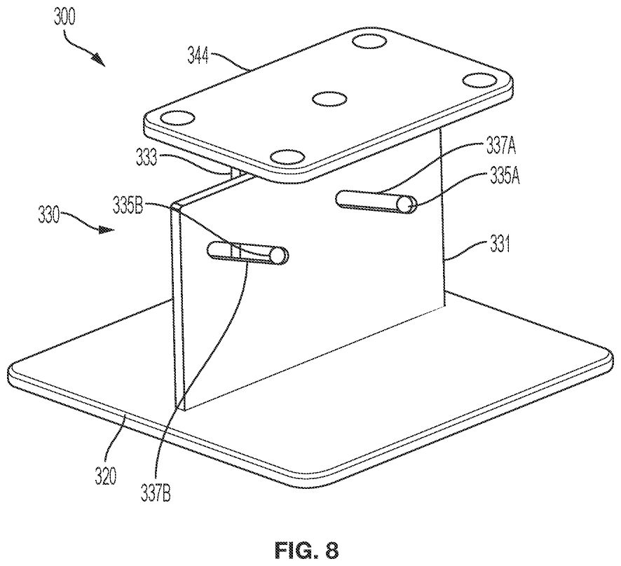

Referring now to FIG. 8 an example stabilizing base 300 for supporting a medical device/system, such as a catheter assembly 114 is shown. The stabilizing base 300 can incorporate any of the features of stabilizing bases disclosed herein and can be made from any suitable material, such as metal or plastic.

The stabilizing base 300 includes a base plate 320, a post 330, and a platform 344. The post 330 comprises a lower portion 331, which extends vertically upwards from the base plate 320, and an upper portion 333, which extends vertically downwards from the platform 344. The platform 344 can removably attach to a rail system (not shown).

The base plate 320 can be a substantially flat plate of any suitable shape and dimension to provide counterbalance and support to the medical device/system secured to the stabilizing base 300. The base plate can be square, round, hexagonal, and the like. The base plate 420 can be solid or can have a void space within the center of the plate. In some implementations, the base plate 320 can be a solid rectangular plate.

The lower portion 331 of the post 330 has a first slot 337A and a second slot 337B. The first and second slots, 337A, 337B are diagonal and set at some distance from one another. The upper portion 333 has a first peg 335A and a second peg 335B that extend outward from the surface of the upper portion that is proximate to the lower portion 331, such that the first peg 335A is disposed within the first slot 337A of the lower portion 331 of the post 330, and the second peg 335B is disposed within the second slot 337B of the lower portion 331 of the post 330. The height of the platform 344 in relation to base plate 320 can be adjusted by slidably moving the first and second pegs 335A, 335B within the first and second slots 335A, 335B. When the first and second pegs 335A, 335B are proximate to lower ends of the first and second slots, 337A, 337B the platform 344 will be closer to the base plate 320. When the first and second pegs 335A, 335B are slid upwards towards upper ends of the first and second slots, 337A, 337B, the platform 344 will move farther away from the base plate 320.

In some implementations, the first and second pegs 335A, 335B can be secured within the first and second slots, 337A, 337B by any suitable method, such as by friction engagement between the slots 337A, 337B and the pegs 335A, 335B or between the upper and lower portions 331, 333 of the post 330. In some implementations, the first and second pegs, 335A, 335B extend through the first and second pegs 335A, 335B and screw into nuts (not shown) which can be tightened or loosen to increase or decrease, respectively, the friction engagement between the upper and lower portions 331, 333 of the post 330.

A rail and/or clamp system (such as the rail and/or clamp systems mentioned above) can be removably attached to the platform 344 of the stabilizing base 300 by any suitable means, such as, for example, with fasteners, threaded fasteners, snaps, clamps, latches, friction fit, spring-loaded clamps, hook and loop fasteners, magnets, or the like. The rail system can be configured to be entirely or partially retained by features of the platform 344 of the stabilizing base 300 so that when the stabilizing base is covered by a sterile barrier (not shown), such as a drape, the rail system can still be attached to the stabilizing base 300. That is, the rail system can be attached to the stabilizing base 300 without attachment means, such as fasteners, that would pierce or puncture the sterile barrier arranged between the platform 344 and the rail system. In some implementations, the rail system can be attached to the stabilizing base 300 using magnetic plates. Example rails that can be used with the stabilizing base 300 are disclosed by U.S. Provisional Patent Application Ser. No. 63/073,392 and/or PCT Application No. PCT/US2021/048333, filed on Aug. 31, 2021.

The base plate 320 can be placed flush against the surface of a table 110 (see FIG. 1), and a patient's legs can rest atop the base plate 320, such that the post 330 extends vertically upwards in between the patient's legs. The position of the delivery system/catheter assembly 114 relative to the patient can be optimized by adjusting the positions of the various elements of the stabilizing base 300, such as by raising and lowering the height of the platform 344 relative to the base plate 320.

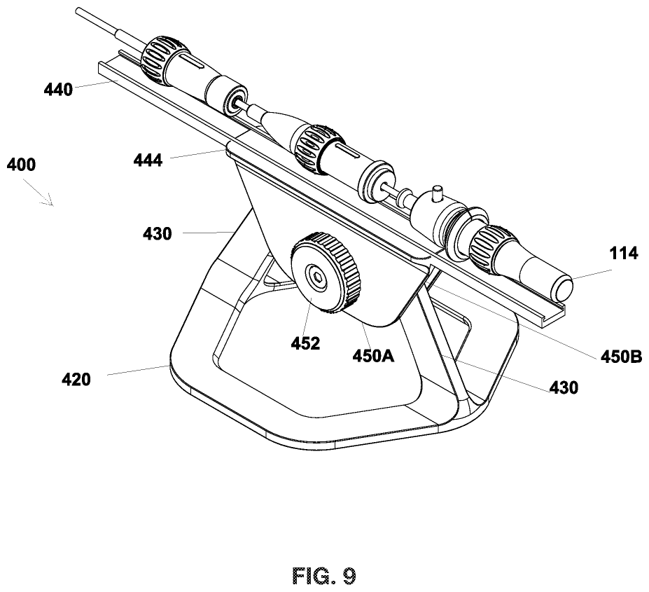

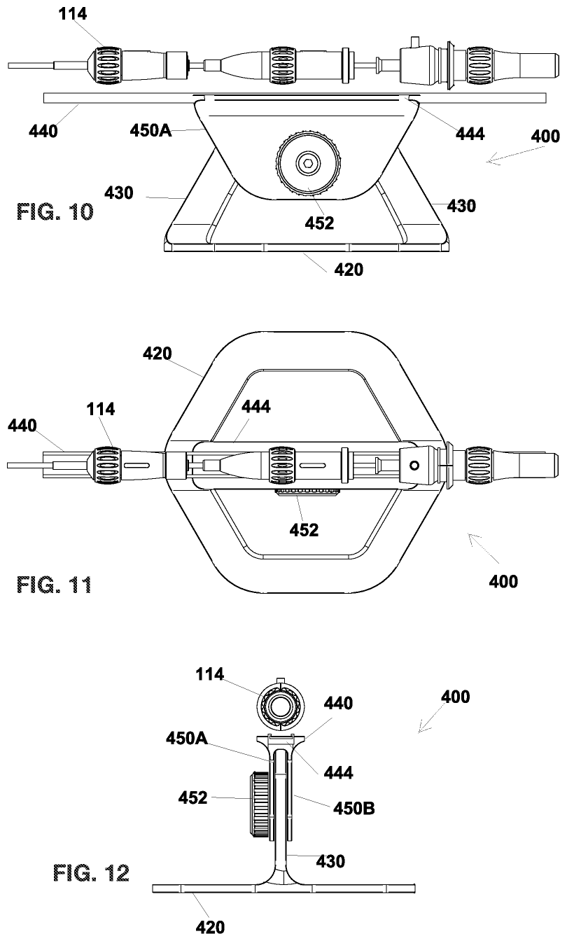

Referring now to FIGS. 9-12, an example stabilizing base 400 for supporting a medical device/system is shown. The stabilizing base 400 can incorporate any of the features of stabilizing bases disclosed herein and can be made from any suitable material, such as metal or plastic.

The stabilizing base 400 includes a base plate 420, a post 430, post plates 450A, 450B, a platform 444, and a rail system 440. The post 430 extends vertically upwards from the base plate 420 and attaches to the platform 444. The platform 444 removably attaches to the rail system 440 (though in some implementations, the rail system can be integrally formed as part of the platform). Clamps can be attached to the rail system 440 to secure the medical device/system, such as a catheter assembly 114, to the rail system 440. The clamps are not shown in FIGS. 9-12 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 400 illustrated by FIGS. 9-12.

The base plate 420 can be a substantially flat plate of any suitable shape and dimension to provide counterbalance and support to the medical device/system secured to the stabilizing base 400. The base plate can be square, round, hexagonal, and the like. The base plate 420 can be solid or can have a void space within the center of the plate. In some implementations, the base plate 420 is a flat, hexagonal frame.

The post 430 can be joined to the base plate 420 by any suitable means, such as welding, an adhesive, or the like. The post 430 can also be molded out of the same material as the base plate 420, such that the post 430 and the base plate 420 are one solid piece. The post 430 can be any suitable shape, such as square, circular, or oblong. In some implementations, the post 430 is arch shaped, such that the post 430 extends vertically from the base plate 420 on a first end and a second end.

The platform 444 can be joined to the post 430 by any suitable means, such as welding, an adhesive, or the like. The platform 444 can also be movably or removably attached to the post 430, such that the position of the platform 444 can be adjusted relative to the post 430. In some implementations, a first post plate 450A and a second post plate 450B parallel thereto, extend vertically downward from the platform 444 on either side of the post 430. The first and second post plates 450A, 450B are such a distance from one another that the post 430 fits tightly within the void space therebetween, but such that the first and second post plates 450A, 450B can still move relative to the post 430. A knob mechanism 452, proximate the first plate 450A, extends through the first plate 450A and attaches to the second plate 450B by any suitable means. When the knob mechanism 452 is tightened, the knob mechanism 452 decreases the distance between the first and second post plates 450A, 450B, thereby increasing the friction engagement between the post plates 450A, 450B and the post 430. When the knob mechanism 452 is loosened, the distance between the first and second post plates 450A, 450B increases, thereby decreasing the friction engagement between the post plates 450A, 450B and the post 430.

In some implementations, a rail system 440 is removably attached to the platform 444 of the stabilizing base 400 by any suitable means, such as, for example, with fasteners, threaded fasteners snaps, clamps, latches, friction fit, spring-loaded clamps, hook and loop fasteners, magnets, or the like. The rail system 440 can be configured to be entirely or partially retained by features of the platform 444 of the stabilizing base 400 so that when the stabilizing base is covered by a sterile barrier (not shown), such as a drape, the rail system 440 can still be attached to the stabilizing base 400. The barriers 112 illustrated herein (see FIG. 1) can be placed between the stabilizing base 400 and the rail system 440. The rail system 440 can be attached to the stabilizing base 400 without attachment means, such as fasteners, that would pierce or puncture the sterile barrier arranged between the platform 444 and the rail system 440. In some implementations, the rail system can be attached to the stabilizing base 400 using magnetic plates. In some implementations, the horizontal position of rail system 440 can be adjusted relative to the platform 444, such as by sliding the rail system 440 forwards and backwards in relation to the platform 444.

The base plate 420 can be placed flush against the surface of a table 110 (see FIG. 1), and a patient's legs can rest atop the base plate 420, such that the post 430 extends vertically upwards in between the patient's legs. In some implementations, when the knob mechanism 452 of the stabilizing base 400 is loosened, the post plates 450A, 450B can move freely in all directions, such as rotationally, translationally, and vertically, in relation to the post 430. Thus, the position of the platform 444 can be adjusted to an optimal distance and radial position relative to the patient. The knob mechanism 452 can then be tightened such that the platform 444, and thus the rail system 440 and delivery system/catheter assembly secured thereto, can be held in the optimal position relative to the patient.

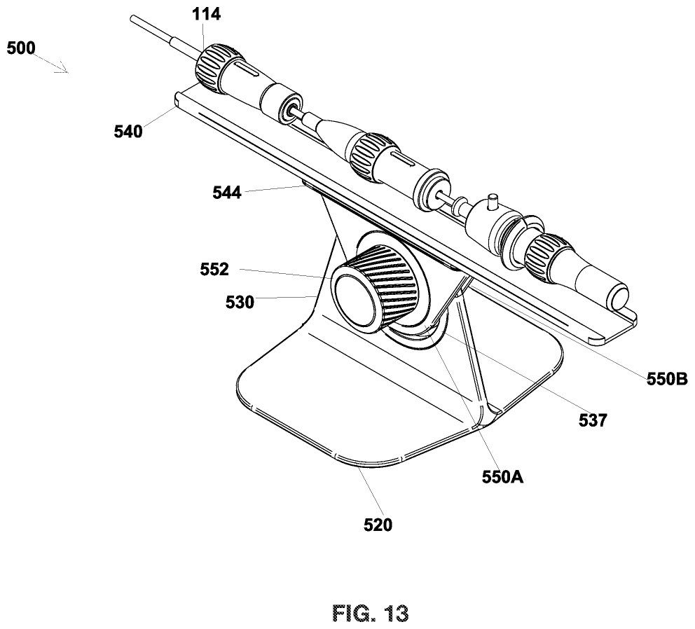

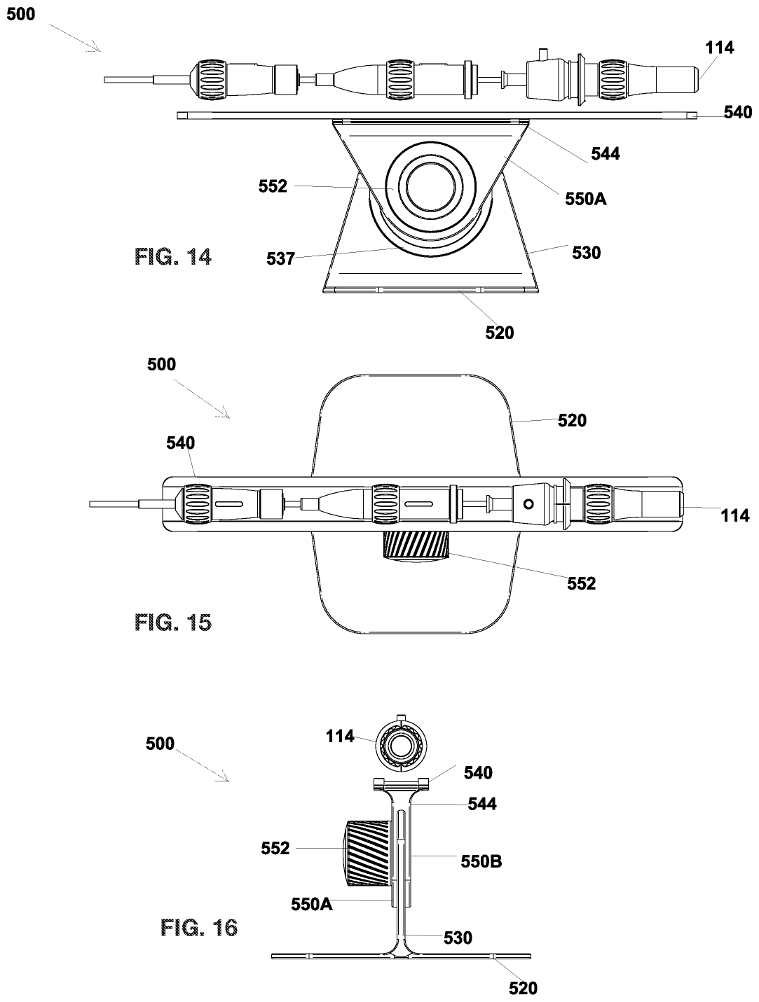

Referring now to FIGS. 13-16, an example stabilizing base 500 for supporting a medical device/system is shown. The stabilizing base 500 can incorporate any of the features of stabilizing bases disclosed herein and can be made from any suitable material, such as metal or plastic.

The stabilizing base 500 includes a base plate 520, a post 530, post plates 550A, 550B, a platform 544, and a rail system 540. The post 530 extends vertically upwards from the base plate 520 and attaches to the platform 544. The platform 544 removably attaches to the rail system 540 (though in some implementations, the rail system and platform can be integrally formed). Clamps can be attached to the rail system 540 to secure the medical device/system, such as a catheter assembly 114, to the rail system 540. The clamps are not shown in FIGS. 13-16 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 500 illustrated by FIGS. 13-16.

The base plate 520 can be a substantially flat plate of any suitable shape and dimension to provide counterbalance and support to the medical device/system secured to the stabilizing base 500. The base plate can be square, round, hexagonal, and the like. The base plate 520 can be solid or can have a void space within the center of the plate. In some implementations, the base plate 520 is a solid, substantially rectangular plate.

The post 530 can be joined to the base plate 520 by any suitable means, such as welding, an adhesive, or the like. The post 530 can also be molded out of the same material as the base plate 520, such that the post 530 and the base plate 520 are one solid piece. The post 530 can be any suitable shape, such as square, circular, or oblong. In some implementations, the post 530 is a flat triangular piece with an orifice 537 through the center of the post. The orifice 537 can be any suitable shape, such as circular, rectangular, or oblong. In some implementations, the orifice 537 is circular.

The platform 544 can be joined to the post 530 by any suitable means, such as welding, an adhesive, or the like. The platform 544 can also be movably or removably attached to the post 530, such that the position of the platform 544 can be adjusted relative to the post 530. In some implementations, a first post plate 550A and a second post plate 550B parallel thereto, extend vertically downward from the platform 544 on either side of the post 530. The first and second post plates 550A, 550B are such a distance from one another that the post 530 fits tightly within the void space therebetween, but such that the first and second post plates 550A, 550B can still move relative to the post 530. A knob mechanism 552, proximate the first plate 550A, extends through the first plate 550A, through the orifice 537 in the post 530, and attaches to the second plate 550B by any suitable means. The edge of the void space or orifice 537 can be made of a high-friction material, such as rubber or silicon, to increase the friction engagement of the post 530 with the first post plate 550A and the second post plate 550B. When the knob mechanism 552 is tightened, the knob mechanism 552 decreases the distance between the first and second post plates 550A, 550B, thereby increasing the friction engagement between the post plates 550A, 550B and the post 530. When the knob mechanism 552 is loosened, the distance between the first and second post plates 550A, 550B increases, thereby decreasing the friction engagement between the post plates 550A, 550B and the post 530.

In some implementations, a rail system 540 is removably attached to the platform 544 of the stabilizing base 500 by any suitable means, such as, for example, with fasteners, threaded fasteners snaps, clamps, latches, friction fit, spring-loaded clamps, hook and loop fasteners, magnets, or the like. The rail system 540 can be configured to be entirely or partially retained by features of the platform 544 of the stabilizing base 500 so that when the stabilizing base is covered by a sterile barrier (not shown), such as a drape, the rail system 540 can still be attached to the stabilizing base 500. That is, the rail system 540 can be attached to the stabilizing base 500 without attachment means, such as fasteners, that would pierce or puncture the sterile barrier arranged between the platform 544 and the rail system 540. In some implementations, the rail system can be attached to the stabilizing base 500 using magnetic plates. The barriers 112 illustrated herein (see FIG. 1) can be placed between the stabilizing base 500 and the rail system 540. In some implementations, the horizontal position of rail system 540 can be adjusted relative to the platform 544, such as by sliding the rail system 540 forwards and backwards in relation to the platform 544.

The base plate 520 can be placed flush against the surface of a table 110 (see FIG. 1), and a patient's legs can rest atop the base plate 520, such that the post 530 extends vertically upwards in between the patient's legs. In some implementations, when the knob mechanism 552 of the stabilizing base 500 is loosened, the post plates 550A, 550B can move freely in all directions, such as rotationally, translationally, and vertically, in relation to the post 530. Thus, the position of the platform 544 can be adjusted to an optimal distance and radial position relative to the patient. The knob mechanism 552 can then be tightened such that the platform 544, and thus the rail system 540 and delivery system/catheter assembly secured thereto, can be held in the optimal position relative to the patient.

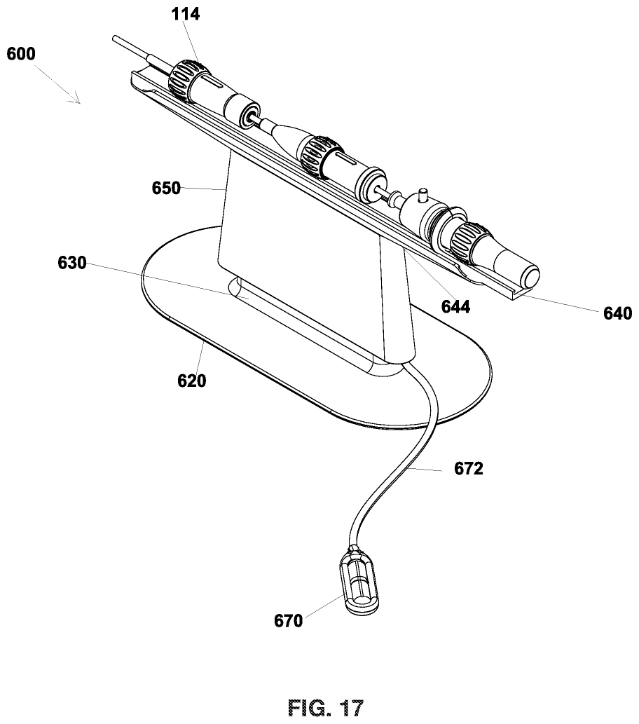

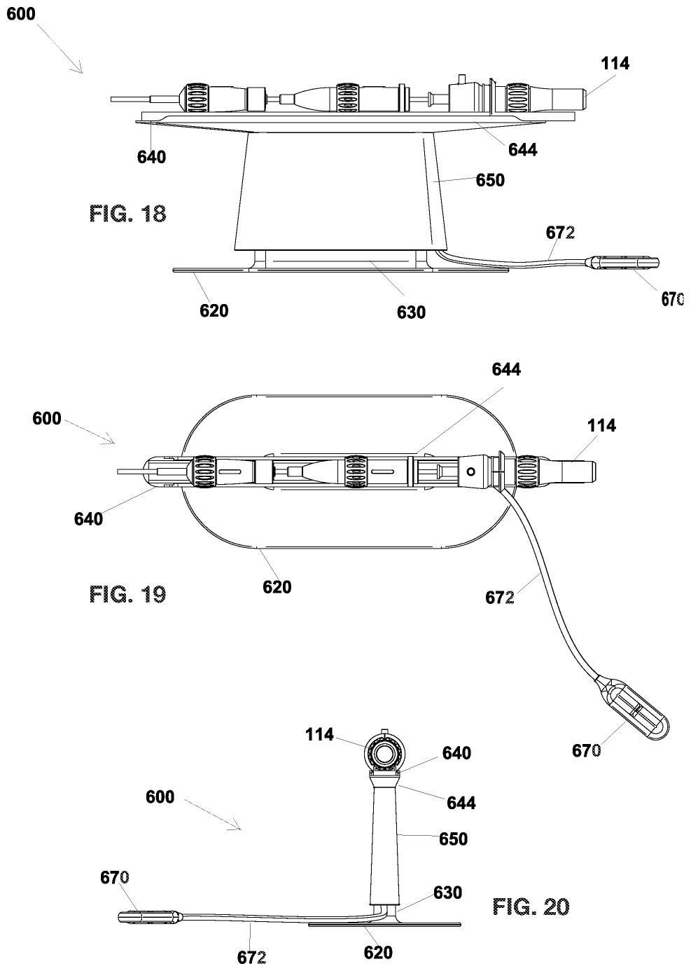

Referring now to FIGS. 17-20, an example stabilizing base 600 for supporting a medical device/system is shown. The stabilizing base 600 can incorporate any of the features of stabilizing bases disclosed herein and can be made from any suitable material, such as metal or plastic.

The stabilizing base 600 includes a base plate 620, a post 630, a post cover 650, a platform 644, and a rail system 640. The post 630 extends vertically upwards from the base plate 620 and attaches to the platform 644. The platform 644 removably attaches to the rail system 640 (though in some implementations, the rail system and platform can be integrally formed). Clamps can be attached to the rail system 640 to secure the medical device/system, such as a catheter assembly 114, to the rail system 640. The clamps are not shown in FIGS. 17-20 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 600 illustrated by FIGS. 17-20.

The base plate 620 can be a substantially flat plate of any suitable shape and dimension to provide counterbalance and support to the medical device/system secured to the stabilizing base 600. The base plate can be square, round, oblong, hexagonal, and the like. The base plate 620 can be solid or can have a void space within the center of the plate. In some implementations, the base plate 620 is a solid, oblong plate.

The post 630 can be joined to the base plate 620 by any suitable means, such as welding, an adhesive, or the like. The post 630 can also be molded out of the same material as the base plate 620, such that the post 630 and the base plate 620 are one solid piece. The post 630 can be any suitable shape, such as square, circular, or oblong. In some implementations, the post 630 is trapezoidal.

The platform 644 can be joined to the post 630 by any suitable means, such as welding, an adhesive, or the like. The platform 644 can also be movably or removably attached to the post 630, such that the position of the platform 644 can be adjusted relative to the post 630. In some implementations, a post cover 650 extends vertically downward from the platform 644 and surrounds the post 630. The area between the post 630 and post cover 650 can house a mechanism (not shown) to control the position of the platform 644 relative to the post 630, such as electromagnetic, electromechanical, hydraulic, pneumatic, gears and the like. The mechanism can be controlled manually and/or electronically. In some implementations, the mechanism is operated by a controller 670, such as a toggle, button, joystick, or the like. The controller 670 can be located anywhere on the stabilizer base 600, such as on the base plate 620, the post 630, or the post cover 650. In some implementations, the controller 670 is remotely attached to the stabilizer base 600 via a cord 672. The controller 670 can also wirelessly operate the mechanism, such as through a computer, tablet, or similar electronic device. The mechanism can control the position of the platform 644 relative to the post 630 by raising and lowering the platform 644, moving the platform 644 forward and backward, changing the pitch of the platform 644 relative to the base plate 620, or shifting the platform 644 left or right.

The rail system 640 is removably attached to the platform 644 of the stabilizing base 600 by any suitable means, such as, for example, with fasteners, threaded fasteners, snaps, clamps, latches, friction fit, spring-loaded clamps, hook and loop fasteners, magnets, or the like. The rail system 640 can be configured to be entirely or partially retained by features of the platform 644 of the stabilizing base 600 so that when the stabilizing base is covered by a sterile barrier (not shown), such as a drape, the rail system 640 can still be attached to the stabilizing base 600. That is, the rail system 640 can be attached to the stabilizing base 600 without attachment means, such as fasteners, that would pierce or puncture the sterile barrier arranged between the platform 644 and the rail system 640. In some implementations, the rail system can be attached to the stabilizing base 600 using magnetic plates. In some implementations, the horizontal position of rail system 640 can be adjusted relative to the platform 644, such as by sliding the rail system 640 forwards and backwards in relation to the platform 644. The barriers 112 illustrated herein (see FIG. 1) can be placed between the stabilizing base 600 and the rail system 640.

The base plate 620 can be placed flush against the surface of a table 110 (as shown in FIG. 1), and a patient's legs can rest atop the base plate 620, such that the post 630 extends vertically upwards in between the patient's legs. In some implementations, the mechanism (not shown) within the post 630 can be controlled, either electronically or manually by the user, to move the platform 644 in all directions, such as rotationally, translationally, and vertically, in relation to the post 630. Thus, the position of the platform 644 can be adjusted to an optimal distance and radial position relative to the patient.

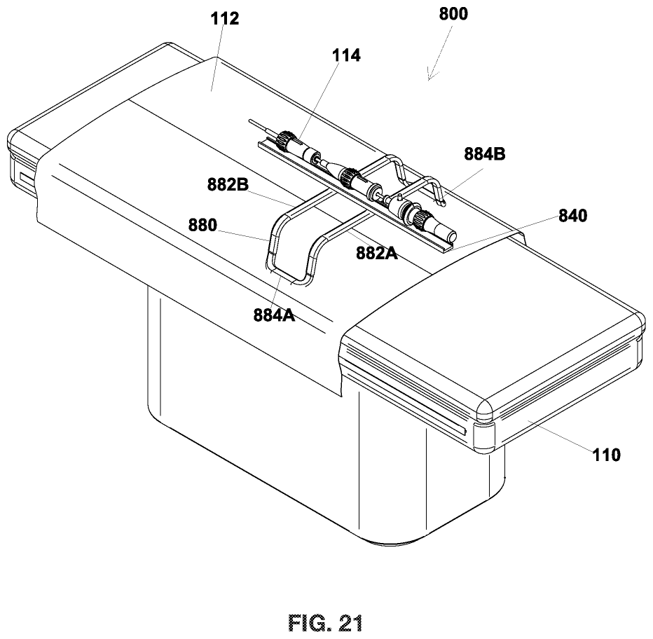

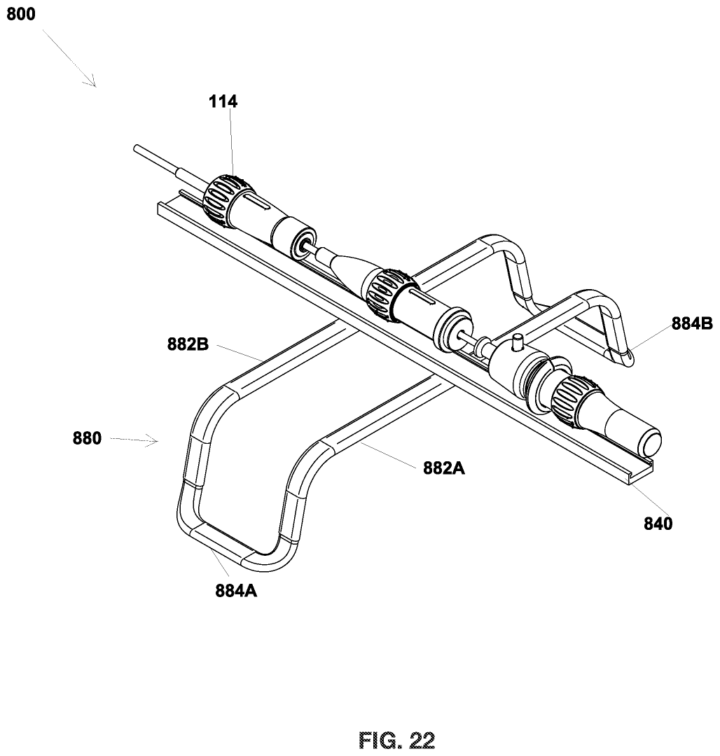

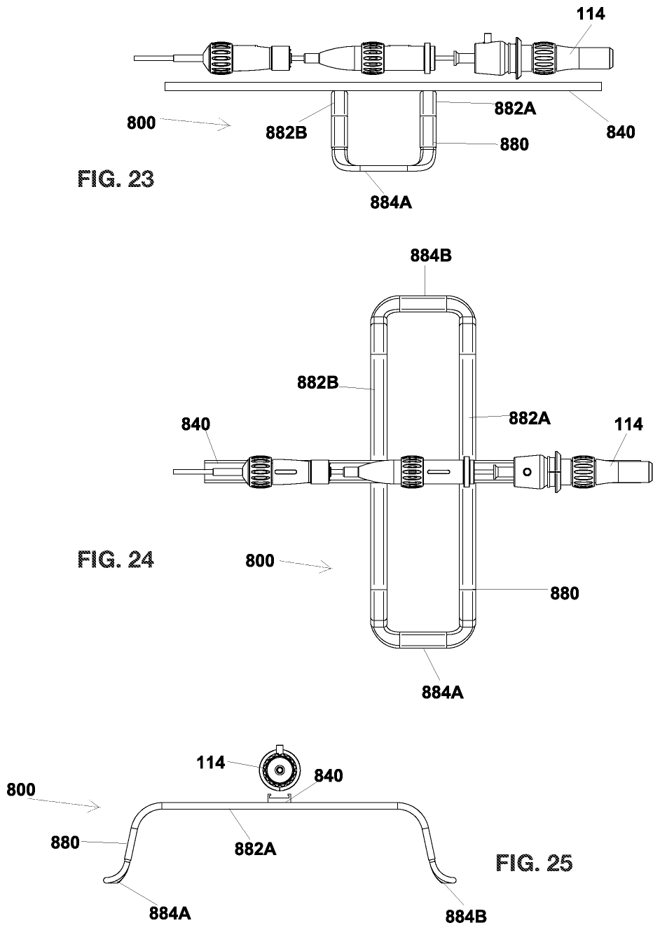

Referring now to FIGS. 21-25, an example stabilizing base 800 for supporting a medical device/system is shown. The stabilizing base 800 can incorporate any of the features of stabilizing bases disclosed herein and can be made from any suitable material, such as metal or plastic.

The stabilizing base 800 includes a frame 880 and a rail system 840. The rail system 840 is movably attached to the top of the frame 880. Clamps can be attached to the rail system 840 to secure the medical device/system, such as a catheter assembly 114, to the rail system 840. The clamps are not shown in FIGS. 21-25 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 800 illustrated by FIGS. 21-25.

The frame 880 has at least one track portion 882A, 882B and at least one support member 884A, 884B. The support members 884A, 884B support and stabilize the frame 880 on a surface, such as a table 110. The support members 884A, 884B can be any suitable shape and configuration, such as posts, bars, triangular or rectangular frames, and the like. The support members 884A, 884B support the at least one track portion 882A, 882B which extend parallel to the surface on which the frame 880 is resting, and at a sufficient height above the surface such that a body of a patient can fit between the surface and the at least one track portion 882A, 882B. The support members 884A, 884B can be attached to the at least one track portion 882A, 882B by any suitable means, either permanently, such as by welding, an adhesive, or the like, or removably, such that the position of the track portions 882A, 882B can be adjusted relative to the support members 884A, 884B.

In some implementations, the frame 880 has a first support member 884A, a second support member 884B, a first track portion 882A, and a second track portion 882B. In some implementations, the first and second support members 884A, 884B are horizontal bars that rest on the surface (e.g. table 110) at opposite ends of the frame 880. One end of the first track portion 882A attaches to one end of the first support member 884A and one end of the second track portion 882B attaches to the other end of the first support member 884A. The other end of the first track portion 882A attaches to one end of the second support member 884B and the other end of the second track portion 882B attaches to the other end of the second support member 884B, forming a U-shape on either end of the frame 880.

In some implementations, the ends of the first and second support members 884A, 884B are recessed within the ends of the first and second track portions 882A, 882B such that the position of the first and second track members 882A, 882B can be vertically adjusted relative to the first and second support members 884A, 884B. Optionally, the ends of the first and second track portions 882A, 882B can be recessed within the ends of the first and second support members 884A, 884B.

The rail system 840 can be joined to the frame 880 by any suitable means, such as welding, or the like. The rail system 840 can also be movably or removably attached to the frame 880, such that the position of the rail system 840 can be adjusted relative to the frame 880. The rail system 840 can be configured to be entirely or partially retained by features of the frame 880 of the stabilizing base 800 such that if the stabilizing base is covered by a sterile barrier (not shown), such as a drape, the rail system 840 can still be attached to the stabilizing base 800. That is, the rail system 840 can be attached to the stabilizing base 800 without attachment means, such as fasteners, that would pierce or puncture the sterile barrier arranged between the frame 880 and the rail system 840. In some implementations, the rail system can be attached to the stabilizing base 800 using magnetic plates. In some implementations, the stabilizing base 800 rests on top of a drape 112. The barriers 112 illustrated herein (see FIG. 1) can be placed between the stabilizing base 800 and the rail system 840. In some implementations, the horizontal position of rail system 840 can be adjusted, such as by sliding the rail system 840 forwards and backwards in relation to the frame 880, or sliding the rail system 840 left to right along the at least one track portion 882A, 882B of the frame 880.

The first and second support members 884A, 884B can be placed flush against the surface of a table 110 (as shown in FIG. 21), and a patient's body can lie underneath the frame 880 of the stabilizing base 800, such that the at least one track portion 882A, 882B are overtop the patient, and the first and second support members 884A, 884B are on either side of the patient. In some implementations, the position of the rail system 840 can be adjusted relative to the frame 880 of the stabilizer base 800, such as by sliding the rail system 840 horizontally or translationally along the at least one track portion 882A, 882B, or pivoting the rail system 840 upwards and downwards. Thus, the position of the rail system 840 can be adjusted to an optimal distance and radial position relative to the patient.

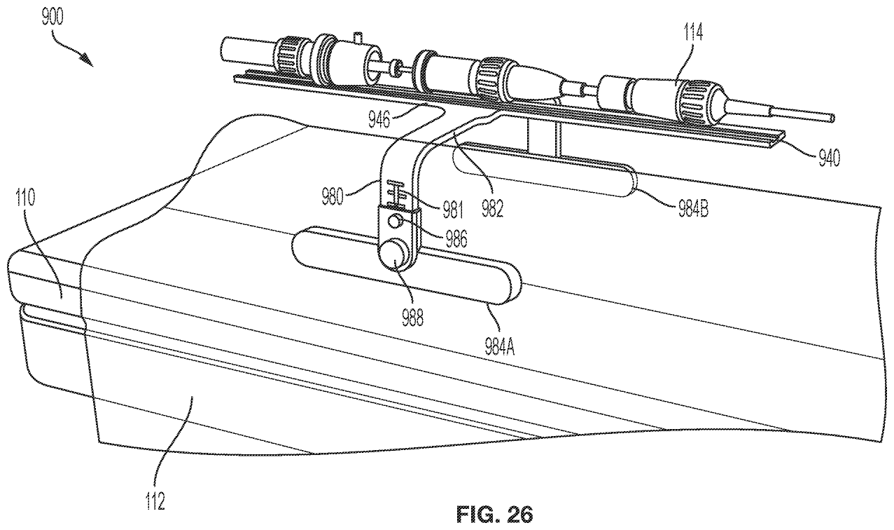

Referring now to FIG. 26, an example stabilizing base 900 for supporting a medical device/system is shown. The stabilizing base 900 can incorporate any of the features of stabilizing bases disclosed herein and can be made from any suitable material, such as metal or plastic.

The stabilizing base 900 includes a frame 980 and a rail system 940. The rail system 940 is movably attached to the top of the frame 980. Clamps can be attached to the rail system 940 to secure the medical device/system, such as a catheter assembly 114, to the rail system 940. The clamps are not shown in FIG. 26 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 900 illustrated by FIG. 26.

The frame 980 has at least one track portion 982 and at least one support member 984A, 984B. The support members 984A, 984B support and stabilize the frame 980 on a surface, such as a table 110. The support members 984A, 984B can be any suitable shape and configuration, such as posts, bars, triangular or rectangular frames, and the like. The support members 984A, 984B support the at least one track portion 982 which extends parallel to the surface on which the frame 980 is resting, and at a sufficient height above the surface such that a body of a patient can fit between the surface and the at least one track portion 982. The support members 984A, 984B can be attached to the at least one track portion 982 by any suitable means, either permanently, such as by welding, an adhesive, or the like, or removably, such that the position of the track portion 982 can be adjusted relative to the support members 984A, 984B.

In some implementations, the frame 980 has a first support member 984A, a second support member 984B, and a track portion 982. In some implementations, the first and second support members 984A, 984B are horizontal bars that rest on the surface (e.g. table 110) at opposite ends of the frame 980. One end of the track portion 982 attaches to the first support member 984A, and the other end of the track portion 982 attaches to the second support member 984B. In some implementations the track portion 982 attaches to the centers of the first and second support members 984A, 984B. In some implementations, the ends of the track portion 982 are recessed within the first and second support members 984A, 984B, such that the track portion 982 can be raised and lowered relative to the first and second support members 984A. In some implementations, the vertical position of the track portion 982 is locked in place unless acted on by a user. In some implementations, a button 986 can be acted on by a force, such as by pressing or pulling, to allow the position of the track portion 982 to be adjusted. When the button 986 is released, the position of the track portion 982 will be locked in place. In some implementations, gradations 981 along at least one side of the track portion 982 indicate the height of the track portion 982 relative to the first and second support members 984A, 984B. In some implementations, the button 986 is located on the track portion 982 or the first and second support members 984A, 984B of the frame 980. In some implementations, the radial position of the track portion 982 relative to the first and second support members 984A, 984B can be adjusted. In some implementations, a knob 988 on at least one end of at least one of the track portions 984A, 984B controls the radial position of the track portion 982. When the knob 988 is acted on by a force, such as pulling, pushing, or turning, the track portion 984 can pivot forward or backwards.

The rail system 940 can be joined to the frame 980 by any suitable means, such as welding, or the like. The rail system 940 can also be movably or removably attached to the frame 980, such that the position of the rail system 940 can be adjusted relative to the frame 980. The rail system 940 can be configured to be entirely or partially retained by features of the frame 980 of the stabilizing base 900 such that if the stabilizing base is covered by a sterile barrier (the illustrated barriers 112 can be moved from between the table 110 and the stabilizing base 900 to between the stabilizing base 900 and the rail system 940 or an additional barrier can be placed between the stabilizing base 900 and the rail system 940), such as a drape, the rail system 940 can still be attached to the stabilizing base 900. That is, the rail system 940 can be attached to the stabilizing base 900 without attachment means, such as fasteners, that would pierce or puncture the sterile barrier arranged between the frame 980 and the rail system 940. In some implementations, the stabilizing base 900 rests on top of a drape 112. In some implementations, the rail system can be attached to the stabilizing base 900 using magnetic plates. In some implementations, the rail system 940 attaches to the track portion 982 with a bracket 946. In some implementations, the horizontal position of rail system 940 can be adjusted relative to the track portion 982 of the frame 980, such as by sliding the rail system 940 forward and backwards in relation to the bracket 946 or frame 980, or sliding the bracket 946 and the rail system 940 left to right along the track portion 982 of the frame 980.

The first and second support members 984A, 984B can be placed flush against the surface of a table 110 (as shown in FIG. 26), and a patient's body can lie underneath the frame 980 of the stabilizing base 900, such that the track portion 982 is overtop the patient, and the first and second support members 984A, 984B are on either side of the patient. In some implementations, the position of the rail system 940 can be adjusted relative to the frame 980 of the stabilizer base 900, such as by sliding the rail system 940 horizontally or translationally along the track portion 982, or pivoting the track portion 982 forwards and backwards in relation to the support members 984A, 984B. Thus, the position of the rail system 940 can be adjusted to an optimal distance and radial position relative to the patient.

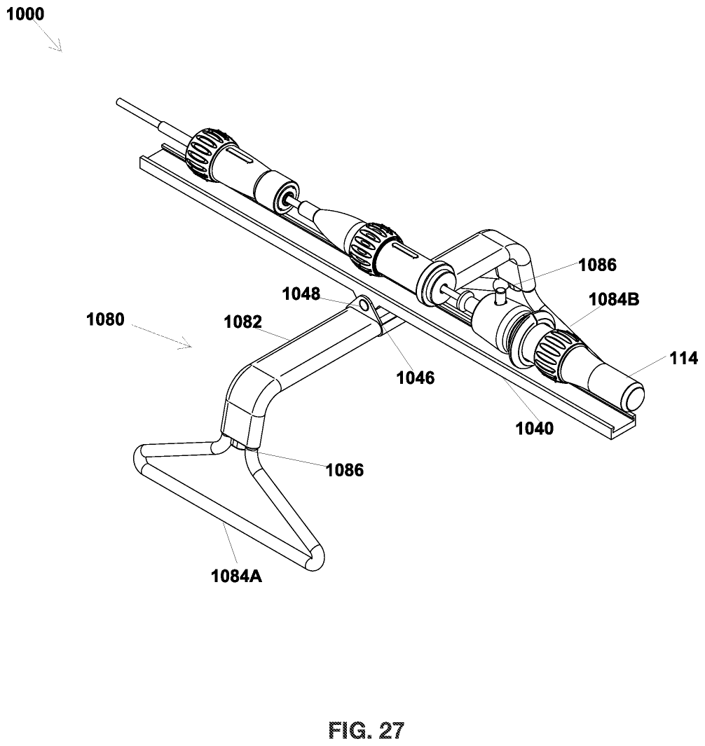

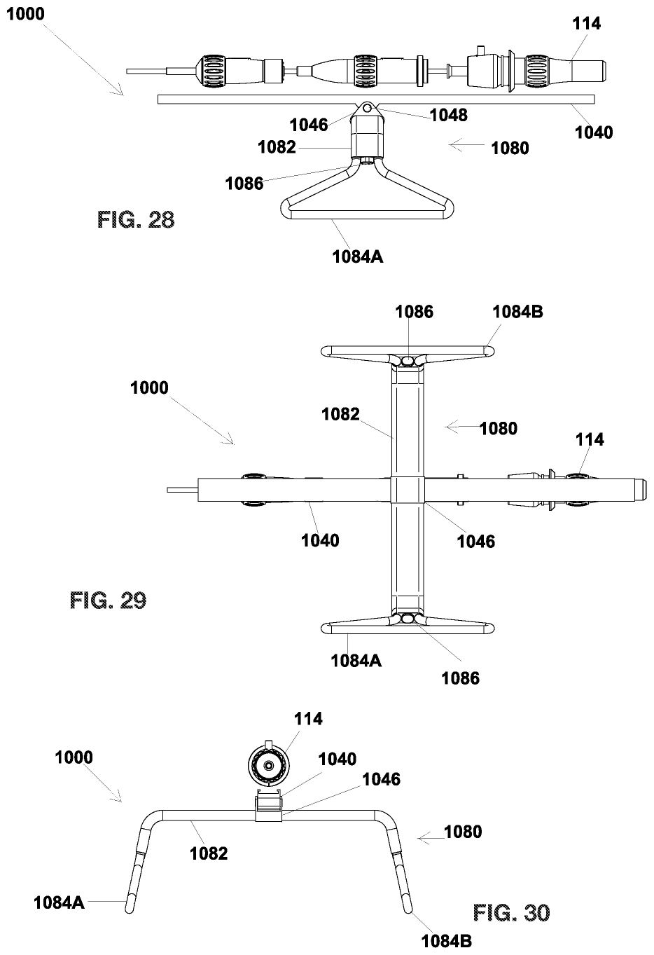

Referring now to FIGS. 27-30, an example stabilizing base 1000 for supporting a medical device/system is shown. The stabilizing base 1000 can incorporate any of the features of stabilizing bases disclosed herein and can be made from any suitable material, such as metal or plastic.

The stabilizing base 1000 includes a frame 1080 and a rail system 1040. The rail system 1040 is movably attached to the top of the frame 1080. Clamps can be attached to the rail system 1040 to secure the medical device/system, such as a catheter assembly 114, to the rail system 1040. The clamps are not shown in FIGS. 27-30 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1000 illustrated by FIGS. 27-30.

The frame 1080 has at least one track portion 1082 and at least one support member 1084A, 1084B. The support members 1084A, 1084B support and stabilize the frame 1080 on a surface, such as a table (not shown). The support members 1084A, 1084B can be any suitable shape and configuration, such as posts, bars, triangular or rectangular frames, and the like. The support members 1084A, 1084B support the at least one track portion 1082 which extends parallel to the surface on which the frame 1080 is resting, and at a sufficient height above the surface such that a body of a patient can fit between the surface and the at least one track portion 1082. The support members 1084A, 1084B can be attached to the at least one track portion 1082 by any suitable means, either permanently, such as by welding, an adhesive, or the like, or removably, such that the position of the track portion 1082 can be adjusted relative to the support members 1084A, 1084B.

In some implementations, the frame 1080 has a first support member 1084A, a second support member 1084B, and a track portion 1082. In some implementations, the first and second support members 1084A, 1084B are triangular frames that rest on the surface (e.g. table 110 in FIG. 1) at opposite ends of the frame 1080. One end of the track portion 1082 attaches to the first support member 1084A, and the other end of the track portion 1082 attaches to the second support member 1084B. In some implementations the track portion 1082 attaches to the centers of the first and second support members 1084A, 1084B. In some implementations, the ends of the first and second support members 1084A, 1084B are recessed with either end of the track portion 1082, such that the track portion 1082 can be raised and lowered relative to the first and second support members 1084A. In some implementations, the vertical position of the track portion 1082 is locked in place unless acted on by a user. In some implementations, a button 1086 can be acted on by a force, such as by pressing or pulling, to allow the position of the track portion 1082 to be adjusted. When the button 1086 is released, the position of the track portion 1082 will be locked in place. In some implementations, optional gradations, or markings along the first and/or second support members 1084A, 1084B can indicate the height of the track portion 1082 relative to the first and second support members 1084A, 1084B. In some implementations, the button 1086 is located on the track portion 1082 or the first and second support members 1084A, 1084B of the frame 1080.

The rail system 1040 can be joined to the frame 1080 by any suitable means, such as welding, or the like. The rail system 1040 can also be movably or removably attached to the frame 1080, such that the position of the rail system 1040 can be adjusted relative to the frame 1080. The rail system 1040 can be configured to be entirely or partially retained by features of the frame 1080 of the stabilizing base 1000 such that if the stabilizing base is covered by a sterile barrier (not shown), such as a drape, the rail system 1040 can still be attached to the stabilizing base 1000. In some implementations, the rail system can be attached to the stabilizing base 1000 using magnetic plates. That is, the rail system 1040 can be attached to the stabilizing base 1000 without attachment means, such as fasteners, that would pierce or puncture the sterile barrier arranged between the frame 1080 and the rail system 1040. A barrier 112 (see FIG. 1) can be placed between the between the stabilizing base 1000 and the rail system 1040. In some implementations, the stabilizing base 1000 rests on top of a drape (not shown). In some implementations, the rail system 1040 attaches to the track portion 1082 with a bracket 1046. In some implementations, the radial position of the rail system 1040 relative to the track portion 1082 can be adjusted. In some implementations, a hinge 1048 on the bracket 1046 controls the radial position of the rail system 1040. In some implementations, the hinge 1048 can be locked in place, such as by a pin, or can be biased towards a certain radial position unless acted on by an outside force. In some implementations, when the hinge 1048 is acted on by a force or when the pin is disengaged, the track portion 1082 can pivot forward or backwards.

In some implementations, the horizontal position of rail system 1040 can be adjusted relative to the track portion 1082 of the frame 1080, such as by sliding the rail system 1040 forward and backwards in relation to the bracket 1046 or frame 1080, or sliding the bracket 1046 and the rail system 1040 left to right along the track portion 1082 of the frame 1080.

The first and second support members 1084A, 1084B can be placed flush against the surface of a table 110 (as shown in FIG. 1), and a patient's body can lie underneath the frame 1080 of the stabilizing base 1000, such that the track portion 1082 is overtop the patient, and the first and second support members 1084A, 1084B are on either side of the patient. In some implementations, the position of the rail system 1040 can be adjusted relative to the frame 1080 of the stabilizing base 1000, such as by sliding the rail system 1040 horizontally or translationally along the track portion 1082, or pivoting the rail system 1040 forwards and backwards in relation to the support members 1084A, 1084B. Thus, the position of the rail system 1040 can be adjusted to an optimal distance and radial position relative to the patient.

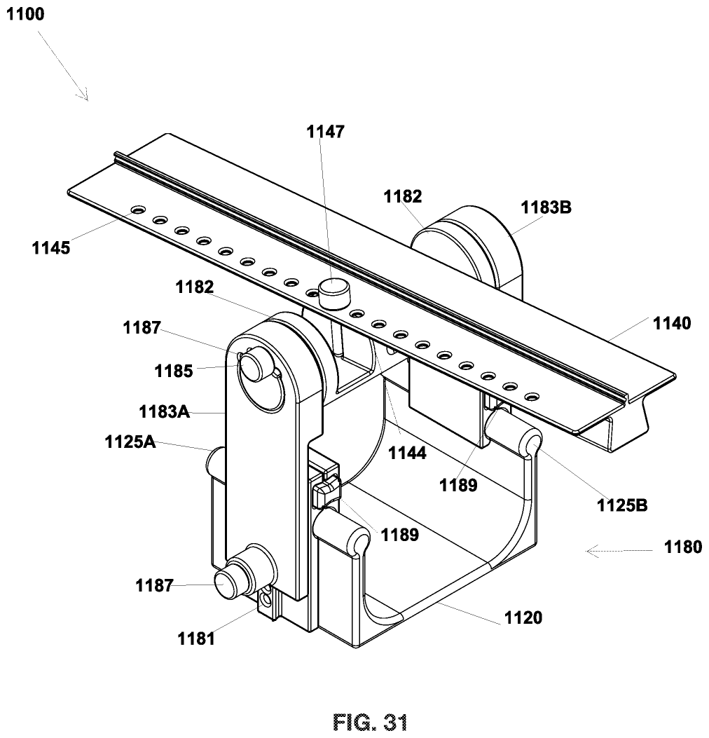

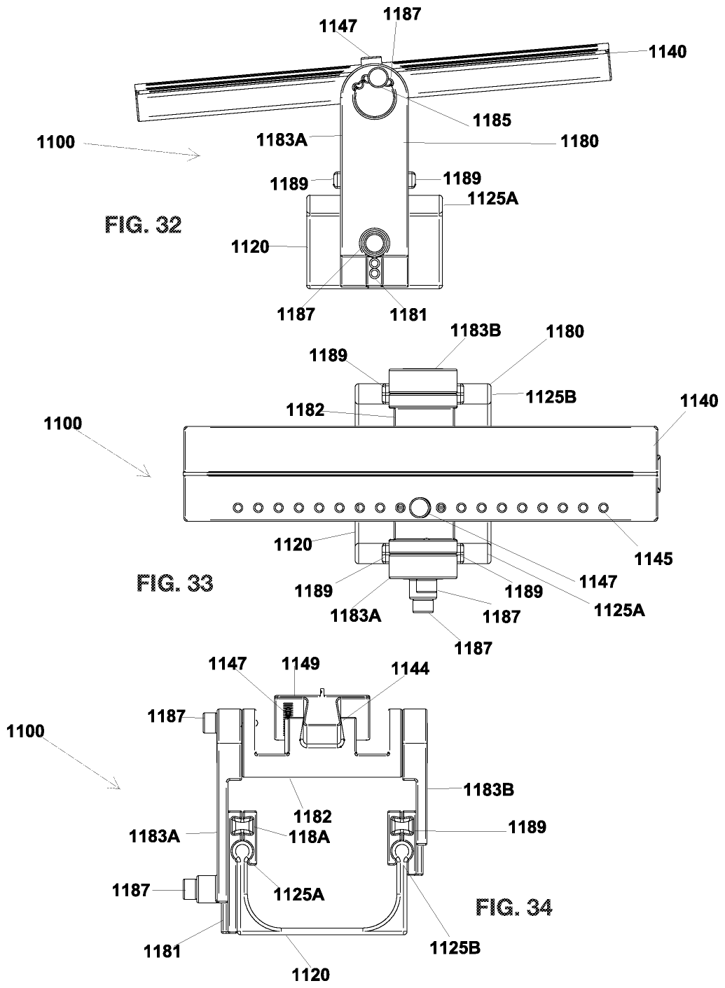

Referring now to FIGS. 31-34, an example stabilizing base 1100 for supporting a medical device/system is shown. The stabilizing base 1100 can incorporate any of the features of stabilizing bases disclosed herein and can be made from any suitable material, such as metal or plastic.

The stabilizing base 1100 includes a base 1120, a frame 1180, and a rail system 1140. The rail system 1140 is movably attached to the frame 1180, and the frame 1180 is movably attached to the base 1120. Clamps (not shown) can be attached to the rail system 1140 to secure the medical device/system, such as a catheter assembly 114 (not shown), to the rail system 1140. The clamps are not shown in FIGS. 31-34 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1100 illustrated by FIGS. 31-34.

The base 1120 can be a substantially flat plate of any suitable shape and dimension to provide counterbalance and support to the medical device/system secured to the stabilizing base 1100. The base 1120 can be square, round, hexagonal, and the like. The base 1120 can be solid or can have a void space within the center of the plate. In some implementations, the base 1120 is a substantially flat plate having a first wall 1125A and a second wall 1125B on either end of the base 1120. In some implementations, the first and second walls 1125A, 1125B attach to the frame 1180 of the stabilizing base 1100. The first and second walls 1125A, 1125B can be attached to the frame 1180 by any suitable means, either permanently, such as by welding, an adhesive, or the like, or removably, such that the position of the frame 1180 can be adjusted relative to the first and second walls 1125A, 1125B of the base 1120. In some implementations, the ends of the first and second walls 1125A, 1125B fit within a void space in the frame 1180, such that the frame 1180 can move relative to the base 1120. In some implementations, the ends of the first and second walls 1125A, 1125B are spherical and the void space within the frame 1180 is spherical such that the frame 1180 can slide forwards and backwards along the spherical edge of the first and second walls 1125A, 1125B. In some implementations, the translational position of the frame 1180 in relation to the base 1120 can be locked and unlocked using at least one actuator 1189, such as a button, switch, tab, or pin. When the at least one actuator 1189 is engaged by a user, the frame 1180 is free to slide in relation to the first and second walls 1125A, 1125B of the frame 1180. In some implementations, multiple actuators 1189 must be engaged at once to release the frame 1180. Once the actuators 1189 are disengaged, the position of the frame 1180 is locked in place relative to the base 1120.

In some implementations, the frame 1180 has at least one track portion 1182, a first arm 1183A and a second arm 1183B. The first and second arms 1183A, 1183B support and stabilize the frame 1180 by attaching to the base 1120. The first and second arms 1183A, 1183B can be any suitable shape and configuration, such as posts, bars, triangular or rectangular frames, and the like. The first and second arms 1183A, 1183B support the at least one track portion 1182 which extends parallel to the base 1120, and at a sufficient height above the base 1120 such that a body of a patient can fit between the base 1120 and the at least one track portion 1182. The first and second arms 1183A, 1183B can be attached to the at least one track portion 1182 by any suitable means, either permanently, such as by welding, an adhesive, or the like, or removably, such that the position of the track portion 1182 can be adjusted relative to the first and second arms 1183A, 1183B.

In some implementations, the first and second arms 1183A, 1183B of the frame 1180 are attached to the base 1120 in a telescoping (vertical adjustment) and/or sliding (longitudinal adjustment) manner. In the illustrated implementation, the height of the first and second arms 1183A, 1183B is adjustable. The arms 1183A, 1183B can be adjustable by any suitable means, such as by hydraulics, gas spring mechanisms, pins, buttons, or the like. In some implementations, at least one of the first and second arms 1183A, 1183B have various orifices 1181 aligned vertically on one side that correspond with incremental heights. A pin 1187 can be inserted into at least one of the first and second arms 1183A, 1183B into one of the various orifices 1181 to lock the height of the frame 1180 in place. In some implementations the track portion 1182 is also adjustable in relation to the first and second arms 1183A, 1183B. The track portion 1182 can be adjustable by any suitable means, such as by hydraulics, gas spring mechanism, pins, buttons, or the like. In some implementations the radial position of the track portion 1182 can be adjusted using various orifices 1185 within at least one of the first and second arms 1183A, 1183B.

The position of the rail system 1140 can be adjusted relative to the track portion 1182 of the frame 1180 by moving the track rail system 1140 translationally forwards and backwards relative to the track portion 1182. The translational position of the rail system 1140 can be adjusted by various means, such as by springs, pins, screws, and the like. In some implementations, the rail system 1140 can have a plurality of orifices 1145 aligned incrementally along the length of the rail system 1140. A pin 1147 can be placed in any one of the plurality of orifices 1145 to hold the rail system 1140 in place in a certain translational position. To move the rail system 1140 forward or backwards, the pin 1147 can be removed from the orifice 1145 and placed in another orifice 1145.

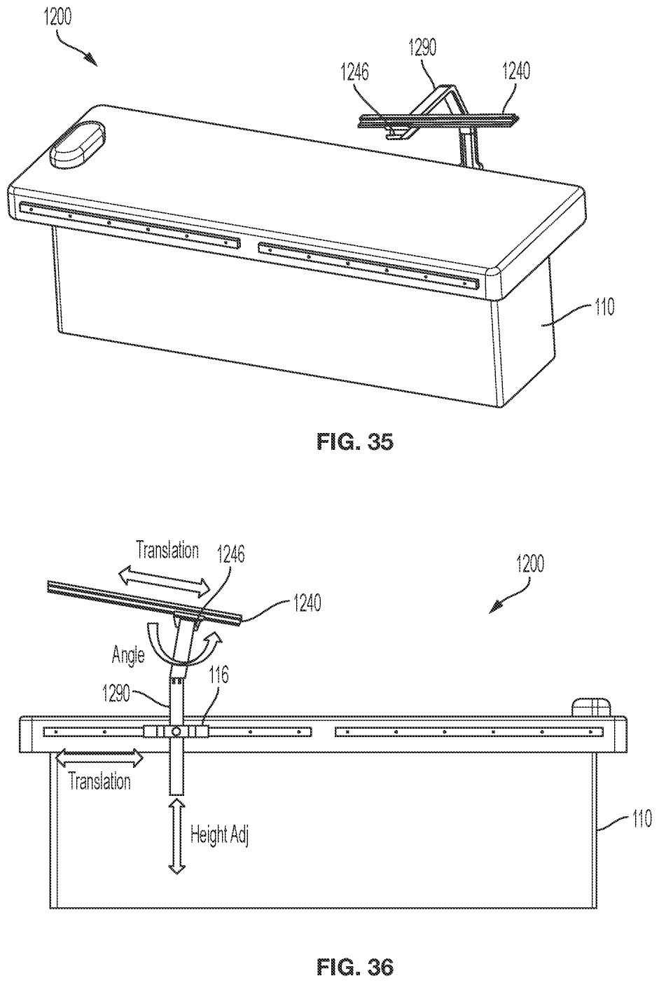

Referring now to FIGS. 35-36, an example stabilizing base 1200 for supporting a medical device/system is shown. The stabilizing base 1200 can incorporate any of the features of stabilizing bases disclosed herein and can be made from any suitable material, such as metal or plastic.

The stabilizing base 1200 includes an arm 1290 and a rail system 1240. The rail system 1240 is attached to the top of the arm 1290. Clamps (not shown) can be attached to the rail system 1240 to secure the medical device/system, such as a catheter assembly 114, to the rail system 1240. The clamps are not shown in FIGS. 35-36 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1200 illustrated by FIGS. 35-36.

The arm 1290 has at least one segment, preferably more than two segments, wherein the segments are movably attached to one another. Optionally, the segments can be attached to one another by any suitable method, such as by welding, screws, or the like. The segments of the arm 1290 can be attached to one another by any suitable method, such as hinged connections, ball joint connections, or telescopic connections, wherein the end of one segment is inserted within the end of another segment. The arm 1290 can be secured to a table 110 by various means, such as by mounting the arm 1290 to the side of the table 110 using a mount 116, or by inserting a portion of the stabilizing base 1200 underneath a portion of the table 110.

The rail system 1240 can be joined to the arm 1290 by any suitable means, such as welding, or the like. The rail system 1240 can also be movably or removably attached to the arm 1290, such that the position of the rail system 1240 can be adjusted relative to the arm 1290. The rail system 1240 can be configured to be entirely or partially retained by features of the arm 1290 of the stabilizing base 1200 such that if the stabilizing base is covered by a sterile barrier (not shown), such as a drape, the rail system 1240 can still be attached to the stabilizing base 1200. That is, the rail system 1240 can be attached to the stabilizing base 1200 without attachment means, such as fasteners, that would pierce or puncture the sterile barrier arranged between the arm 1290 and the rail system 1240. The barriers 112 illustrated herein (see FIG. 1) can be placed between the stabilizing base 1200 and the rail system 1240. In some implementations, the stabilizing base 1200 extends above the drape (not shown). In some implementations, the rail system 1240 attaches to the arm 1290 with a bracket 1246. In some implementations, the radial position of the rail system 1240 relative to the arm 1290 can be adjusted. In some implementations, the translational position of rail system 1240 can be adjusted relative to the arm 1290, such as by sliding the rail system 1240 forwards and backwards in relation to the bracket 1246. Additionally, the bracket 1246 and the rail system 1240 can slide left to right along the arm 1290.

The patient's body can lie substantially underneath the arm 1290 of the stabilizing base 1200, such that the rail system 1240 extends overtop the patient. In some implementations, the position of the rail system 1240 can be adjusted relative to the patient and the surface of the table 110, such as by sliding the rail system 1240 translationally within the bracket 1246, sliding the rail system 1240 left or right along the arm 1290, pivoting the rail system 1240 forwards and backwards. The position of the stabilizing base 1200 can be adjusted in relation to the table 110, such as by raising and lowering the height of the arm 1290 or translating the stabilizing base 1200 forward and backward using the mount 116 attached to the table 110. Thus, the position of the rail system 1240 can be adjusted to an optimal distance and radial position relative to the patient.

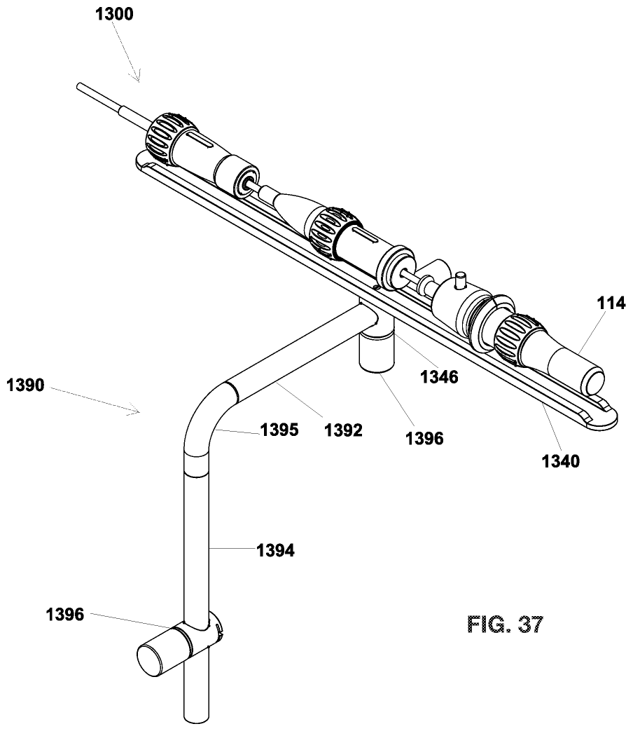

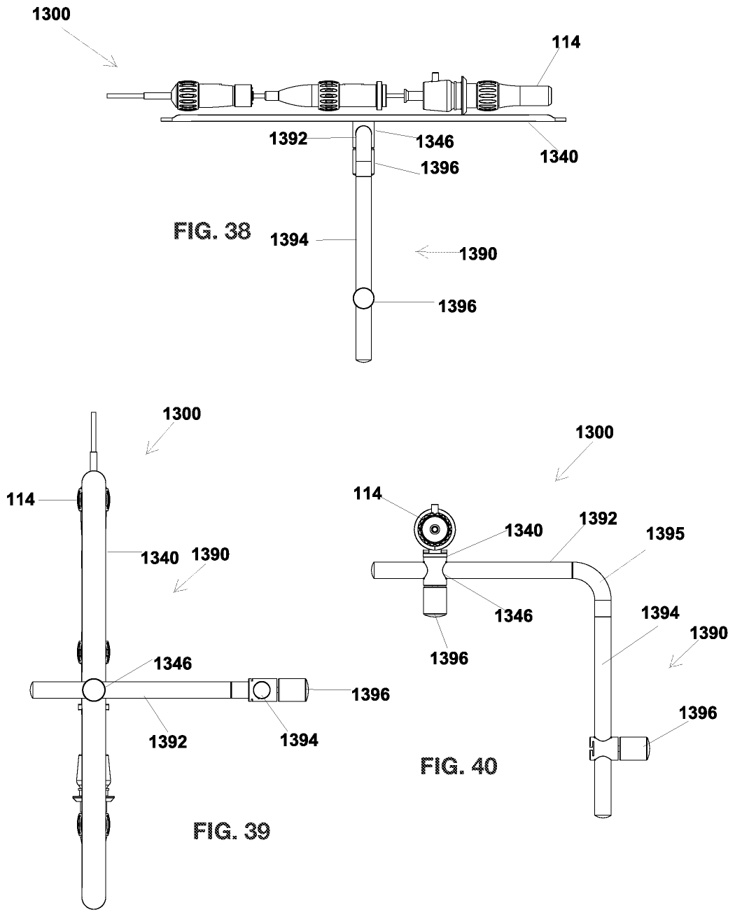

Referring now to FIGS. 37-40, an example stabilizing base 1300 for supporting a medical device/system is shown. The stabilizing base 1300 can incorporate any of the features of stabilizing bases disclosed herein and can be made from any suitable material, such as metal or plastic.

The stabilizing base 1300 includes an arm 1390 and a rail system 1340. The rail system 1340 is attached to the top of the arm 1390. Clamps can be attached to the rail system 1340 to secure the medical device/system, such as a catheter assembly 114, to the rail system 1340. The clamps are not shown in FIGS. 37-40 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1200 illustrated by FIGS. 37-40.

The arm 1390 has at least one segment, preferably more than two segments, wherein the segments are movably attached to one another. Optionally, the segments can be attached to one another by any suitable method, such as by welding, screws, or the like. The segments of the arm 1390 can be attached to one another by any suitable method, such as hinged connections, ball joint connections, or telescopic connections, wherein the end of one segment is inserted within the end of another segment. The arm 1390 can be secured to a table (not shown) by various means, such as by mounting the arm 1390 to the side of the table using a mount (not shown), or by inserting a portion of the stabilizing base 1300 underneath a portion of the table. In some implementations, the arm 1390 comprises a vertical segment 1394, a horizontal segment 1392, and a joint segment 1395. The segments can be any suitable shape or size, such as circular, rectangular, or oblong posts. In some implementations, the horizontal and vertical segments 1392, 1394 are hollow, cylindrical posts. The joint segment 1395 is substantially L-shaped, having a vertical portion and a horizontal portion. The vertical segment 1394 can be telescopically connected to the vertical portion of the joint segment 1395, such that the joint segment 1395 is received within an end of the vertical segment 1394. The horizontal segment 1392 can be telescopically connected to the horizontal portion of the joint segment 1395, such that the joint segment 1395 is received within an end of the horizontal segment 1392. In some implementations, the height of the rail system 1340 relative to the table (not shown) can be adjusted by moving the joint portion 1395 upwards or downwards within the vertical portion 1394. In some implementations, the horizontal position of the rail system 1340 relative to the table (not shown) can be adjusted by moving the joint portion 1395 inwards and outwards within the horizontal portion 1392. In yet another example implementation, the vertical segment 1394, joint segment 1395, and horizontal segment 1392 are permanently attached to one another by any suitable means, such as by screws, welding, adhesive, or the like. In said implementations, the position of the rail system 1340 relative to the table (not shown) can be adjusted using an actuator 1396 attached to the vertical segment 1394 of the arm 1390. The actuator 1396 can be a button, a pull, a knob, or the like. In some implementations, the actuator 1396 is a knob, wherein when the knob is twisted in a direction, such as clockwise, friction engagement between the actuator 1396 and the arm 1390 increases such that the arm 1390 is unable to move relative to the actuator 1396. When the actuator 1396 is twisted in the opposite direction, such as counterclockwise, friction engagement between the arm 1390 and the actuator 1396 decreases such that the arm 1390 is free to move up and down relative to the actuator 1396

The rail system 1340 can be joined to the arm 1390 by any suitable means, such as welding, or the like. The rail system 1340 can also be movably or removably attached to the arm 1390, such that the position of the rail system 1340 can be adjusted relative to the arm 1390. The rail system 1340 can be configured to be entirely or partially retained by features of the arm 1390 of the stabilizing base 1300 such that if the stabilizing base is covered by a sterile barrier (not shown), such as a drape, the rail system 1340 can still be attached to the stabilizing base 1300. That is, the rail system 1340 can be attached to the stabilizing base 1300 without attachment means, such as fasteners, that would pierce or puncture the sterile barrier arranged between the arm 1390 and the rail system 1340. The barriers 112 illustrated herein (see FIG. 1) can be placed between the stabilizing base 1300 and the rail system 1340. In some implementations, the stabilizing base 1300 extends above a drape (not shown). In some implementations, the rail system 1340 attaches to the arm 1390 with a bracket 1346. In some implementations, the radial position of the rail system 1340 relative to the arm 1390 can be adjusted. In some implementations, the translational position of rail system 1340 can be adjusted relative to the arm 1390, such as by sliding the rail system 1340 forward and backwards in relation to the bracket 1346. Additionally, the bracket 1346 and the rail system 1340 can slide left to right along the arm 1390. In some implementations, the position of the bracket 1346 can be locked and unlocked using an actuator 1396. The actuator 1396 can be a button, a pull, a knob, or the like. In some implementations, the actuator 1396 is a knob, wherein when the knob is twisted in a direction, such as clockwise, friction engagement between the bracket 1346 and the rail system 1340 increases such that the bracket 1346 is unable to move relative to the rail system 1340. When the actuator 1396 is twisted in the opposite direction, such as counterclockwise, friction engagement between the bracket 1346 and the rail system 1340 decreases such that the bracket 1346 is free to move left and right relative to the rail system 1340.

The patient's body can lie substantially underneath the arm 1390 of the stabilizing base 1300, such that the rail system 1340 extends overtop the patient. In some implementations, the position of the rail system 1340 can be adjusted relative to the patient and the surface of the table (not shown), such as by sliding the rail system 1340 translationally within the bracket 1346, sliding the rail system 1340 left or right along the arm 1390, and/or pivoting the rail system 1340 forwards and backwards. The position of the stabilizing base 1300 can be adjusted in relation to the table (not shown), such as by raising and lowering the height of the arm 1390 or translating the stabilizing base 1300 forward and backward along the table. Thus, the position of the rail system 1340 can be adjusted to an optimal distance and radial position relative to the patient.

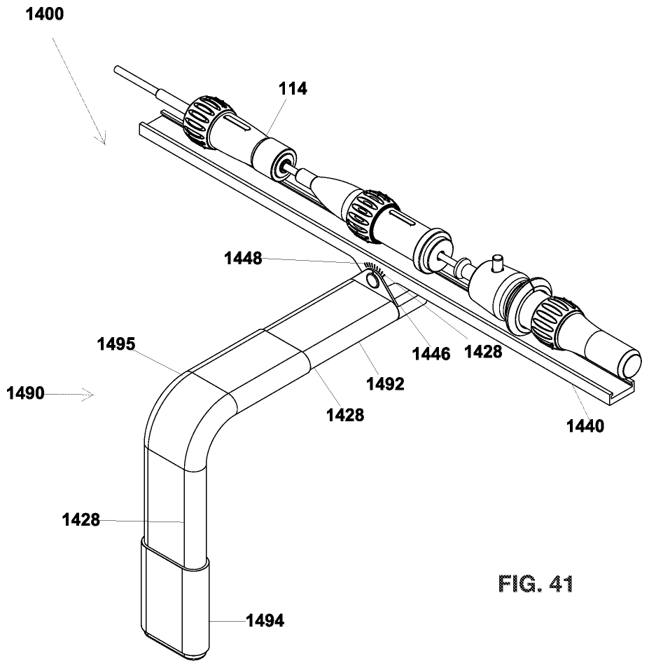

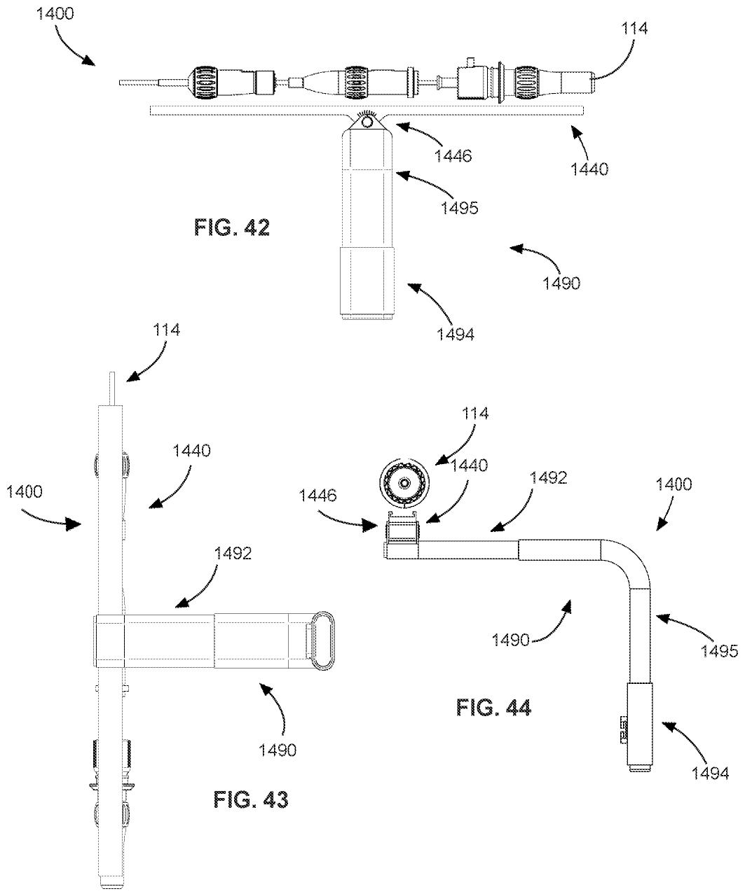

Referring now to FIGS. 41-44, an example stabilizing base 1400 for supporting a medical device/system is shown. The stabilizing base 1400 can incorporate any of the features of stabilizing bases disclosed herein and can be made from any suitable material, such as metal or plastic.

The stabilizing base 1400 includes an arm 1490 and a rail system 1440. The rail system 1440 is attached to the top of the arm 1490. Clamps can be attached to the rail system 1440 to secure the medical device/system, such as a catheter assembly 114, to the rail system 1440. The clamps are not shown in FIGS. 41-44 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1400 illustrated by FIGS. 41-44.

The arm 1490 has at least one segment, preferably more than two segments, wherein the segments are movably attached to one another. Optionally, the segments can be attached to one another by any suitable method, such as by welding, screws, or the like. The segments of the arm 1490 can be attached to one another by any suitable method, such as hinged connections, ball joint connections, or telescopic connections, wherein the end of one segment is inserted within the end of another segment. The arm 1490 can be secured to a table by various means, such as by mounting the arm 1490 to the side of the table using a mount, or by inserting a portion of the stabilizing base 1400 underneath a portion of the table. In some implementations, the arm 1490 comprises a vertical segment 1494, a horizontal segment 1492, and a joint segment 1495. The segments can be any suitable shape or size, such as circular, rectangular, or oblong posts. In some implementations, the horizontal and vertical segments 1492, 1494 are hollow, oblong posts. The joint segment 1495 is substantially L-shaped, having a vertical portion and a horizontal portion. The vertical segment 1494 can be telescopically connected to the vertical portion of the joint segment 1495, such that the joint segment 1495 is received within an end of the vertical segment 1494. The horizontal segment 1492 can be telescopically connected to the horizontal portion of the joint segment 1495, such that the joint segment 1495 is received within an end of the horizontal segment 1492. In some implementations, the height of the rail system 1440 relative to the table (not shown) can be adjusted by moving the joint portion 1495 upwards or downwards within the vertical segment 1494. In some implementations, the horizontal position of the rail system 1440 relative to the table (not shown) can be adjusted by moving the joint portion 1495 inwards and outwards within the horizontal portion 1492. In some implementations, gradation 1428 marked in increments along one or more of the segments can be used to indicate the position of the vertical and horizontal segments 1494, 1492 relative to the joint segment 1495.

The rail system 1440 can be joined to the arm 1490 by any suitable means, such as welding, or the like. The rail system 1440 can also be movably or removably attached to the arm 1490, such that the position of the rail system 1440 can be adjusted relative to the arm 1490. The rail system 1440 can be configured to be entirely or partially retained by features of the arm 1490 of the stabilizing base 1400 such that if the stabilizing base is covered by a sterile barrier (not shown), such as a drape, the rail system 1440 can still be attached to the stabilizing base 1400. That is, the rail system 1440 can be attached to the stabilizing base 1400 without attachment means, such as fasteners, that would pierce or puncture the sterile barrier arranged between the arm 1490 and the rail system 1440. The barriers 112 illustrated herein (see FIG. 1) can be placed between the stabilizing base 1400 and the rail system 1440. In some implementations, the stabilizing base 1400 extends above the drape (not shown). In some implementations, the rail system 1440 attaches to the arm 1490 with a bracket 1446. In some implementations, the radial position of the rail system 1440 relative to the arm 1490 can be adjusted. In some implementations, the bracket 1446 has a hinged portion 1448 that allows the position of the rail system 1440 to move radially forwards and backwards. In some implementations, the hinge 1448 can be unlocked, to allow for the adjustment of the radial position of the rail system 1440 relative to the arm 1490, and the hinge 1448 can be locked to prevent further movement of the rail system 1440 once the desired radial position has been established. In some implementations, the hinge 1448 can be tuned or selected, such as by a spring or counterweight mechanism, to prevent radial movement of the rail system 1440 unless the hinge 1448 is acted upon by applying a sufficient upwards or downwards force to the rail system 1440.

In some implementations, the translational position of rail system 1440 can be adjusted relative to the arm 1490, such as by sliding the rail system 1440 forwards and backwards in relation to the bracket 1446. Additionally, the bracket 1446 and the rail system 1440 can slide left to right along the arm 1490. In some implementations, the position of the bracket 1446 can be locked and unlocked using an actuator (not shown). The actuator can be a button, a pull, a knob, or the like.

The patient's body can lie substantially underneath the arm 1490 of the stabilizing base 1400, such that the rail system 1440 extends overtop the patient. In some implementations, the position of the rail system 1440 can be adjusted relative to the patient and the surface of the table 110, such as by sliding the rail system 1440 translationally within the bracket 1446, sliding the rail system 1440 left or right along the arm 1490, and/or pivoting the rail system 1440 forwards and backwards. The position of the stabilizing base 1400 can be adjusted in relation to the table 110, such as by raising and lowering the height of the arm 1490 or translating the stabilizing base 1400 forward and backward along a table. Thus, the position of the rail system 1440 can be adjusted to an optimal distance and radial position relative to the patient.

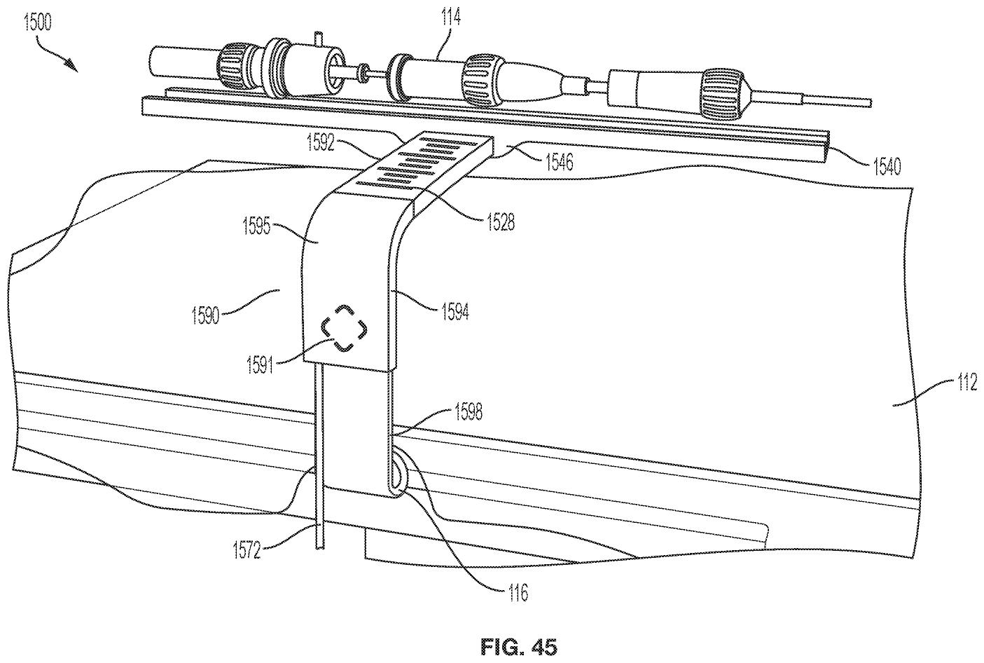

Referring now to FIGS. 45-48, an example stabilizing base 1500 for supporting a medical device/system is shown. The stabilizing base 1500 can incorporate any of the features of stabilizing bases disclosed herein and can be made from any suitable material, such as metal or plastic.

The stabilizing base 1500 includes an arm 1590 and a rail system 1540. The rail system 1540 is attached to the top of the arm 1590. Clamps can be attached to the rail system 1540 to secure the medical device/system, such as a catheter assembly 114, to the rail system 1540. The clamps are not shown in FIGS. 45-48 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1500 illustrated by FIGS. 45-48.

The arm 1590 has at least one segment, preferably more than two segments, wherein the segments are movably attached to one another. The segments can be attached to one another by any suitable method, such as by welding, screws, or the like. Additionally, the segments of the arm 1590 can be attached to one another by any suitable method, such as hinged connections, ball joint connections, or telescopic connections, wherein the end of one segment is inserted within the end of another segment. The arm 1590 can be secured to a table 110 by various means, such as by mounting the arm 1590 to the side of the table using a mount 116, or by inserting a stabilizing portion 118 of the stabilizing base 1500 underneath a portion of the table 110. In some implementations, the arm 1590 comprises a vertical segment 1594, a horizontal segment 1592, and a joint segment 1595.

The segments can be any suitable shape or size, such as circular, rectangular, or oblong posts. In some implementations, the horizontal and vertical segments 1592, 1594 are solid, flat rectangular posts. The joint segment 1595 is substantially L-shaped, having a vertical portion and a horizontal portion. The vertical segment 1594 can be telescopically connected to the vertical portion of the joint segment 1595, such that the joint segment 1595 receives an end of the vertical segment 1594. The horizontal segment 1592 can be telescopically connected to the horizontal portion of the joint segment 1595, such that the joint segment 1595 receives an end of the horizontal segment 1592.

In some implementations, the height of the rail system 1540 relative to the table (not shown) can be adjusted by moving the vertical segment 1594 upwards or downwards within the joint segment 1595. In some implementations, the horizontal position of the rail system 1540 relative to the table 110 can be adjusted by moving the horizontal segment 1592 inwards and outwards within the joint segment 1595. In some implementations, gradation 1528 marked in increments along one or more of the segments can be used to indicate the position of the vertical and horizontal segments 1594, 1592 relative to the joint segment 1595. The vertical and horizontal positions of the arm segments can be controlled by various means, such as springs, gas springs, hydraulics, and the like.

The arm 1590 can house a mechanism (not shown). The mechanism can be controlled manually and/or electronically. In some implementations, the mechanism is operated by controls 1591, such as toggles, buttons, a joystick, or the like. The controls 1591 can be located anywhere on the stabilizer base 1500, such as on arm 1590. In some implementations, the controls 1591 are remotely attached to the stabilizer base 1500 via a cord 1572. The controls 1591 can also wirelessly operate the mechanism, such as through a computer, tablet, or similar electronic device. The mechanism can control the positions of the vertical segment 1594 and horizontal segment 1592 of the arm 1590 relative to the joint segment 1595 by raising and lowering the joint segment 1595, moving the horizontal segment 1592 inwards and outwards. The mechanism can also change the pitch of the rail system 1540 relative to the arm 1590 or shift the rail system 1540 forwards and backwards.

The rail system 1540 can be joined to the arm 1590 by any suitable means, such as welding, or the like. The rail system 1540 can also be movably or removably attached to the arm 1590, such that the position of the rail system 1540 can be adjusted relative to the arm 1590. The rail system 1540 can be configured to be entirely or partially retained by features of the arm 1590 of the stabilizing base 1500 such that if the stabilizing base is covered by a sterile barrier 112, such as a drape, the rail system 1540 can still be attached to the stabilizing base 1500. That is, the rail system 1540 can be attached to the stabilizing base 1500 without attachment means, such as fasteners, that would pierce or puncture the sterile barrier arranged between the arm 1590 and the rail system 1540. The barriers 112 illustrated herein (see FIG. 1) can be placed between the stabilizing base 1500 and the rail system 1540. In some implementations, the stabilizing base 1500 extends above the drape 112. In some implementations, the rail system 1540 attaches to the arm 1590 with a bracket 1546. In some implementations, the radial position of the rail system 1540 relative to the arm 1590 can be adjusted. In some implementations, the bracket 1546 has a hinged portion that allows the position of the rail system 1540 to move radially forward and backwards. In some implementations, the hinge can be tuned or selected, such as by a spring or counterweight mechanism, to prevent radial movement of the rail system 1540 unless the hinge is acted upon by applying a sufficient upward or downward force to the rail system 1540.

In some implementations, the translational position of rail system 1540 can be adjusted relative to the arm 1590, such as by sliding the rail system 1540 forward and backwards in relation to the bracket 1546. Additionally, the bracket 1546 and the rail system 1540 can slide left to right along the arm 1590. In some implementations, the position of the bracket 1546 can be locked and unlocked using an actuator. The actuator can be a button, a pull, a knob, or the like.

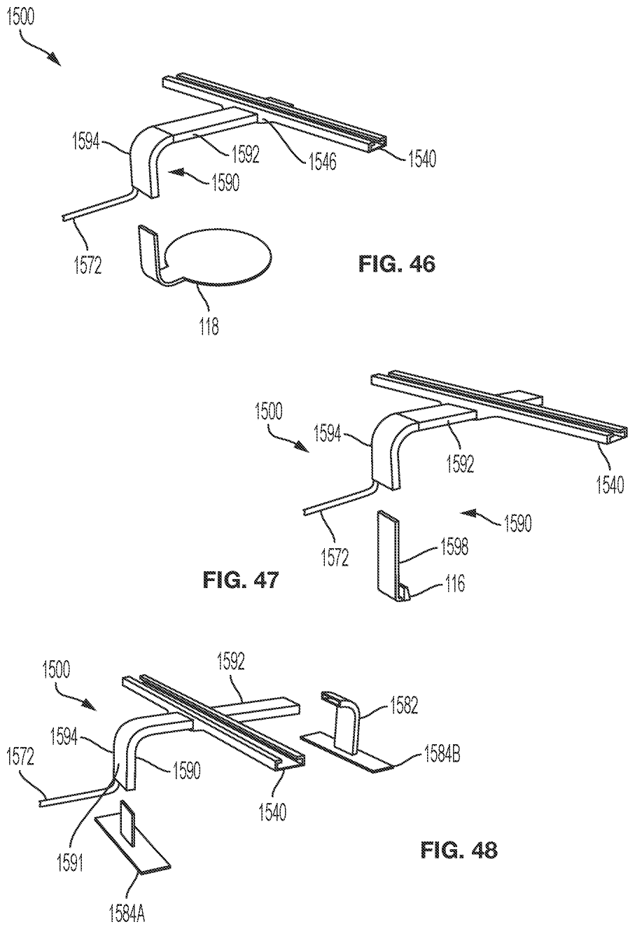

In some implementations, the stabilizing base 1500 can include interchangeable base portions or mounting devices, such that the stabilizing base 1500 can be secure to or on a surface, such as a table 110, in a variety of ways. FIG. 46 shows a stabilizing base 1500 with an optional stabilizer 118, which can be placed in between surfaces, such as upper and lower portions of a table 110. The weight of the upper portion of a table 110 in combination with the weight of a patient lying thereon, will create a sufficient downward force to secure the stabilizing base 1500 in place.

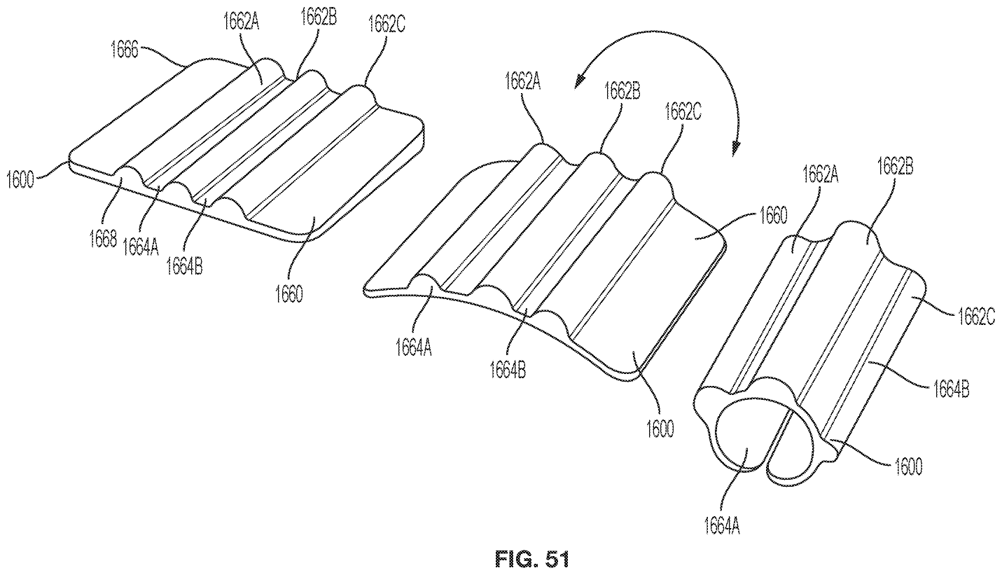

FIG. 47 shows the stabilizing base 1500 with a mount 116 as shown in FIG. 51 and described above. FIG. 48 depicts the stabilizing base 1500 with a first support member 1584A and a second support member 1584B. The first support member 1584A can be removably attached to the vertical segment 1594 of the arm 1590 and the second support member 1584B can be removably attached to the horizontal segment 1592 of the arm 1590. In this implementation, the stabilizing base 1500 can rest on the surface of a table 110.

The patient's body can lie substantially underneath the arm 1590 of the stabilizing base 1500, such that the rail system 1540 extends overtop the patient. In some implementations, the position of the rail system 1540 can be adjusted relative to the patient and the surface of the table 110, such as by sliding the rail system 1540 translationally within the bracket 1546, sliding the rail system 1540 left or right along the arm 1590, and/or pivoting the rail system 1540 forward and backwards. The position of the stabilizing base 1500 can be adjusted in relation to the table 110, such as by raising and lowering the height of the arm 1590 or translating the stabilizing base 1500 forward and backward along the table 110. Thus, the position of the rail system 1540 can be adjusted to an optimal distance and radial position relative to the patient.

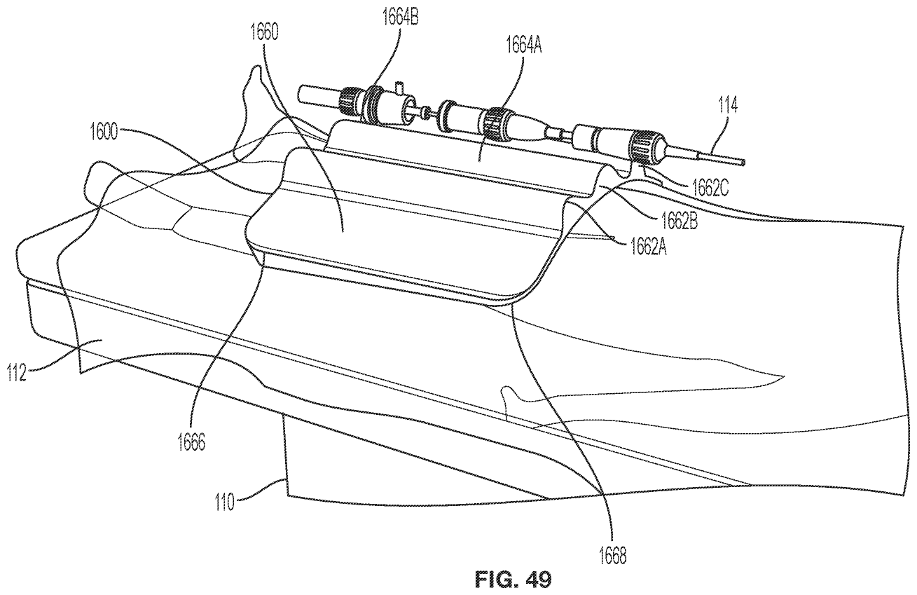

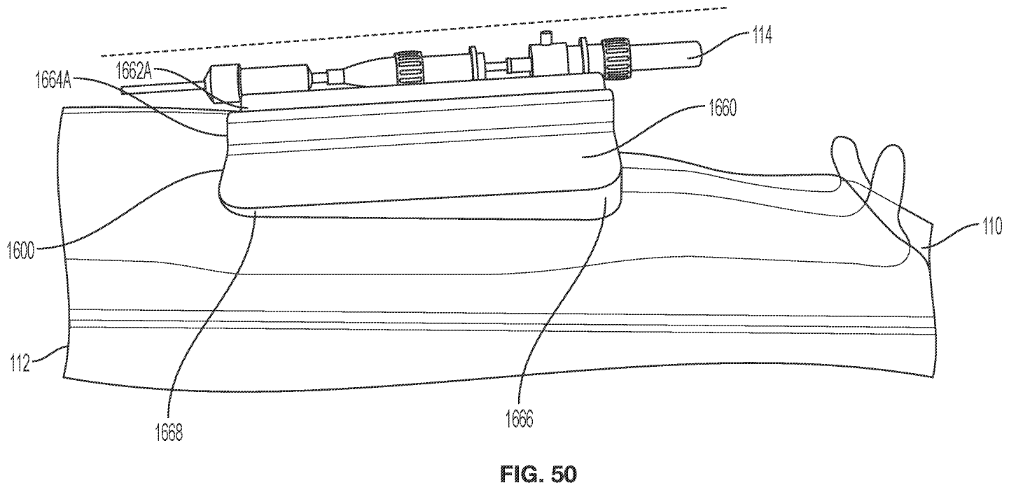

Referring now to FIGS. 49-51, an example stabilizing base 1600 for supporting a medical device/system is shown. The stabilizing base 1600 can incorporate any of the features of stabilizing bases disclosed herein and can be made from any suitable material, such as metal, plastic, or silicon.

The stabilizing base 1600 comprises a mat 1660. A medical device/system, such as a catheter assembly 114, can be placed directly on top of the mat 1660 or be secured by some other means, such as to a rail system (not shown) attached to the mat 1660. In some implementations, the mat 1660 has a distal end 1666 and a proximal end 1668, wherein the proximal end 1668 is closest to the point of entry into a patient. The mat 1660 can be tapered or angled such that the thickness of the distal end 1666 is greater than that of the proximal end 1668. Thus, when the delivery system/catheter assembly 114 engages with the mat 1660 of the stabilizing base 1600, the delivery system/catheter assembly 114 will be angled towards the point of entry into a patient.

In some implementations, the surface of the mat 1660 can have features, such as indentations, ridges, valleys, and the like, to secure the position of the catheter assembly 114 relative to the stabilizing base 1600. In some implementations, the mat 1660 has a plurality of ridges 1662A, 1662B, 1662C extending parallel to one another from the proximal end 1668 to the distal end 1666 of the mat 1660. The ridges 1662A, 1662B, 1662C form at least one valley 1664A, 1664B therebetween. The catheter assembly 114 can be placed within a valley 1664A, 1664B, in between two of the ridges 1662A, 1662B, 1662C wherein the ridges 1662A, 1662B, 1662C will prevent the catheter assembly 114 from moving left or right in relation to the stabilizing base 1600.

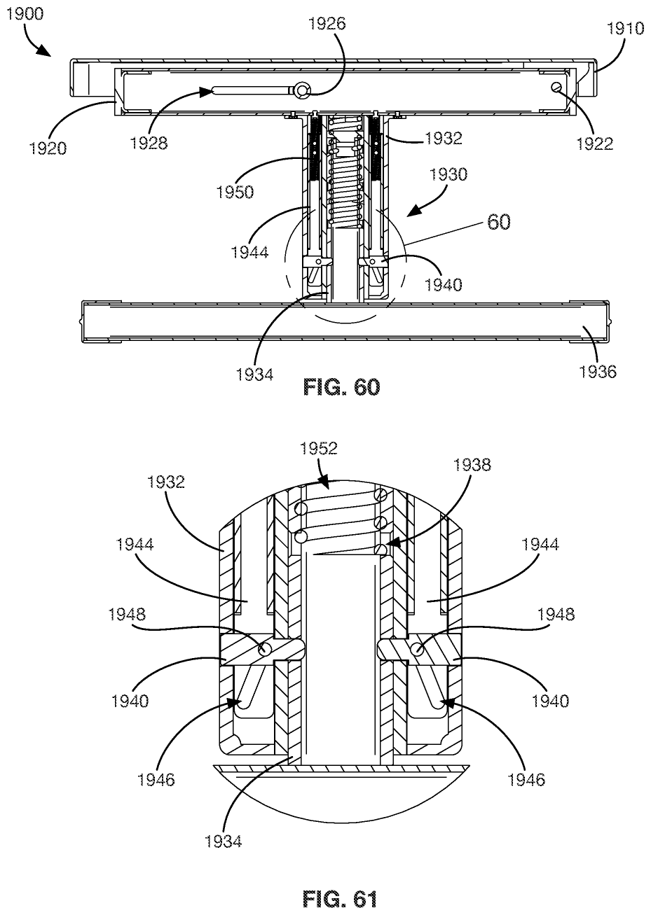

The mat 1660 can be made of any suitable material, such as plastic, rubber, or silicone. As shown in FIG. 60, In some implementations the mat is made of a flexible material, such as silicone, that can bend and roll for easy storage and placement on a variety of different shaped surfaces and patients. The mat 1660 material, such as silicone, can also increase the friction engagement between the catheter assembly 114 and the stabilizing base 1600 such that the friction engagement prevents the catheter assembly 114 from moving forwards and backwards in relation to the stabilizing base.

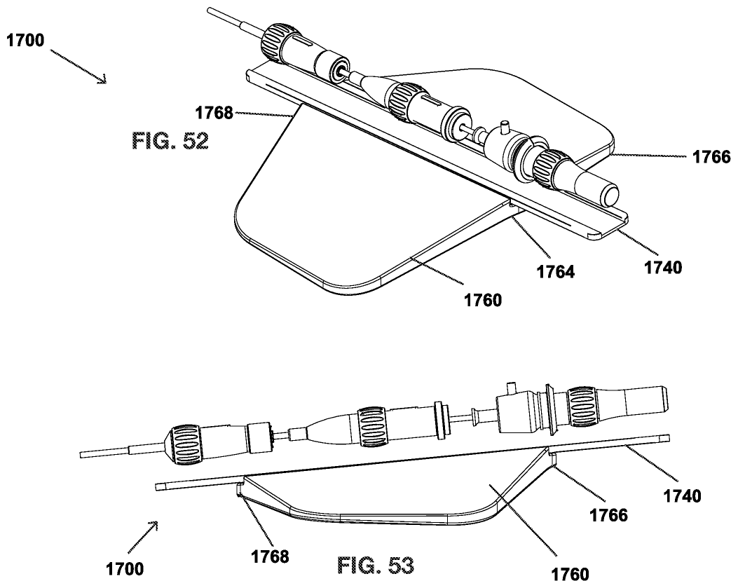

Referring now to FIGS. 52-54, an example stabilizing base 1700 for supporting a medical device/system is shown. The stabilizing base 1700 can incorporate any of the features of stabilizing bases disclosed herein and can be made from any suitable material, such as metal, plastic, or silicon.

The stabilizing base 1700 comprises a mat 1760. A medical device/system, such as a catheter assembly 114, can be placed directly on top of the mat 1760 or be secured by some other means, such as to a rail system 1740 attached to the mat 1760. In some implementations, the mat 1760 has a distal end 1766 and a proximal end 1768, wherein the proximal end 1768 is closest to the point of entry into a patient. The mat 1760 can be tapered or angled such that the thickness of the distal end 1766 is greater than that of the proximal end 1768. Thus, when the delivery system/catheter assembly 144 engages with the mat 1760 of the stabilizing base 1700, the delivery system/catheter assembly 114 will be angled towards the point of entry into a patient.

In some implementations, the surface of the mat 1760 can have features, such as indentations, ridges, valleys, and the like, to secure the position of the catheter assembly 114 relative to the stabilizing base 1700. In some implementations, the mat 1760 has a valley 1764 extending from the proximal end 1768 to the distal end 1766 of the mat 1760. In some implementations, the catheter assembly 114 can be placed directly within the valley 1764 to prevent the catheter assembly 114 from moving left or right in relation to the stabilizing base 1700. Optionally, a rail system 1740 can be inserted into the valley 1764, and the catheter assembly 114 can be secured to the rail system 1740 by any suitable means, such as clamps. The clamps are not shown in FIGS. 52 and 53-48 to simplify the drawings, but can be the clamps of any of the implementations described herein. Any of the clamps, features, and/or rails disclosed by U.S. Provisional Patent Application Ser. No. 63/073,392 or other applications incorporated herein can be used on the stabilizing base 1700 illustrated by FIGS. 52-54.

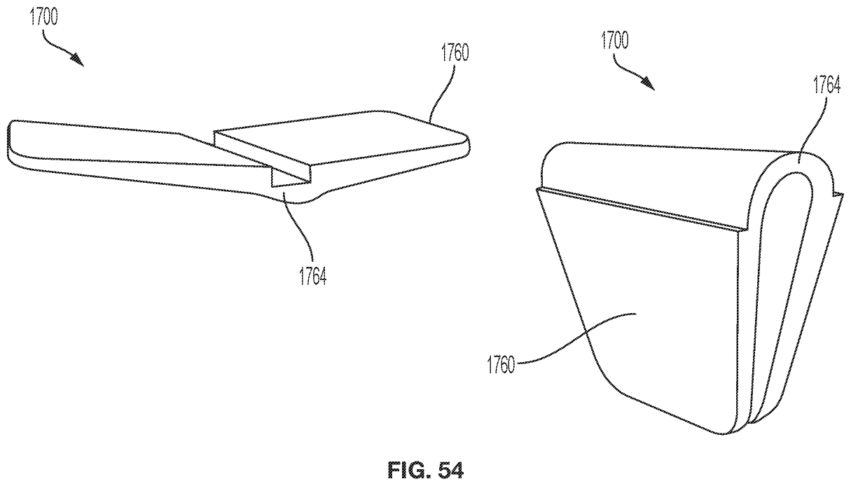

The mat 1760 can be made of any suitable material, such as plastic, rubber, or silicone. As shown in FIG. 54, In some implementations the mat is made of a flexible material, such as silicone, that can bend or roll for easy storage and placement on a variety of different shaped surfaces and patients. The mat 1760 material, such as silicone, can also increase the friction engagement between the catheter assembly 114 and the stabilizing base 1700 such that the friction engagement prevents the catheter assembly 114 from moving forward and backwards in relation to the stabilizing base.

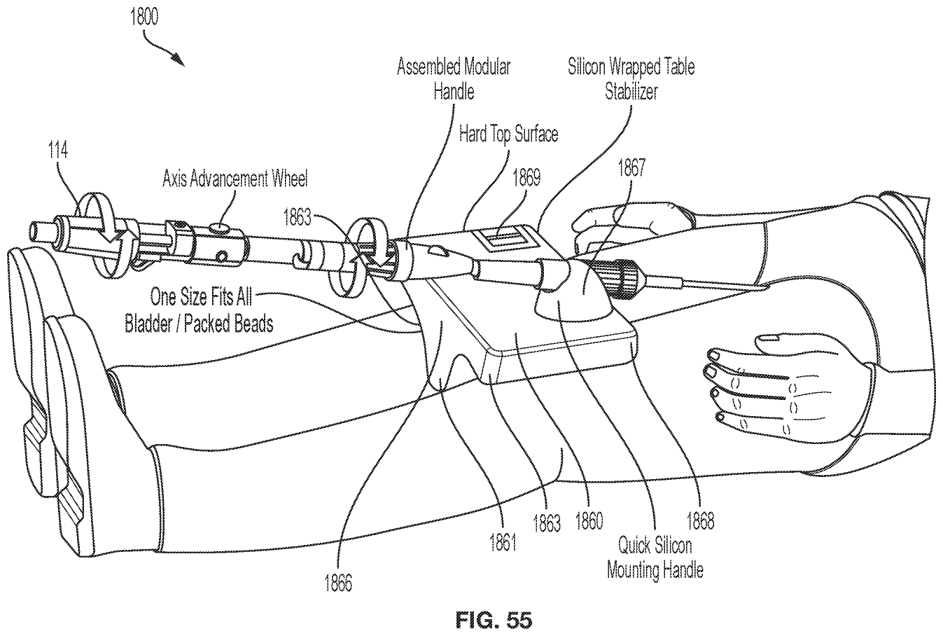

Referring now to FIG. 55, an example stabilizing base 1800 for supporting a medical device/system is shown. The stabilizing base 1800 can incorporate any of the features of stabilizing bases disclosed herein and can be made from any suitable material, such as metal, plastic, or silicon.

The stabilizing base 1800 comprises a mat 1860. A medical device/system, such as a catheter assembly 114, can be placed directly on top of the mat 1860 or be secured by some other means, such as to a rail system (not shown) attached to the mat 1860, or by a handle mount 1867. The illustrated handle mount is sized and shaped to hold the handle of the delivery system/catheter assembly in place, but allow the handle to be positioned (e.g. advanced, retracted, rotated). Once the handle is released again, the handle mount 1867 maintains the new position of the handle.

In some implementations, the mat 1860 has a distal end 1866 and a proximal end 1868, wherein the proximal end 1868 is closest to the point of entry into a patient. The mat 1860 can also have a ridge 1861 along a bottom surface 1863 extending from the distal end 1866 to the proximal end 1868 of the mat 1860. The ridge 1861 can rest between a patient's legs in order to hold the mat 1860 in place relative to the patient. The mat 1860 can be solid or it can have an outer material and an inner material. The inner material can be pliable, such as packed beads, air, liquid, or a semi-solid material, that allows the mat to conform to a variety of surfaces and patients. The outer surface of the mat 1860 can be made of silicon, rubber, or another flexible material. In some implementations, the mat 1860 can have a solid top 1869 to better support the delivery system/catheter assembly 114 secured thereon.

In some implementations, the delivery system or catheter assembly 114 is secured to the mat 1860 using a handle mount 1867. The handle mount 1867 can be made of any suitable material, such as metal, rubber, silicone, plastic, or the like. The handle mount 1867 can be a clasp, bracket, or similar configuration. In some implementations, the handle mount 1867 has a semi-annular shape having a gap or opening for receiving a portion of the catheter assembly 114, such as a body of the handle. The handle of the catheter assembly 114 can rotate, move translationally proximally and distally towards and away from the patient within the handle mount 1867. Optionally, the handle mount 1867 can move relative to the mat 1860, such that the mounting handle 1867 can rotate frontward and backwards, left to right, and side to side relative to the mat 1860. Thus, the position of the catheter assembly 114 can be adjusted to an optimal distance and radial position relative to the patient.

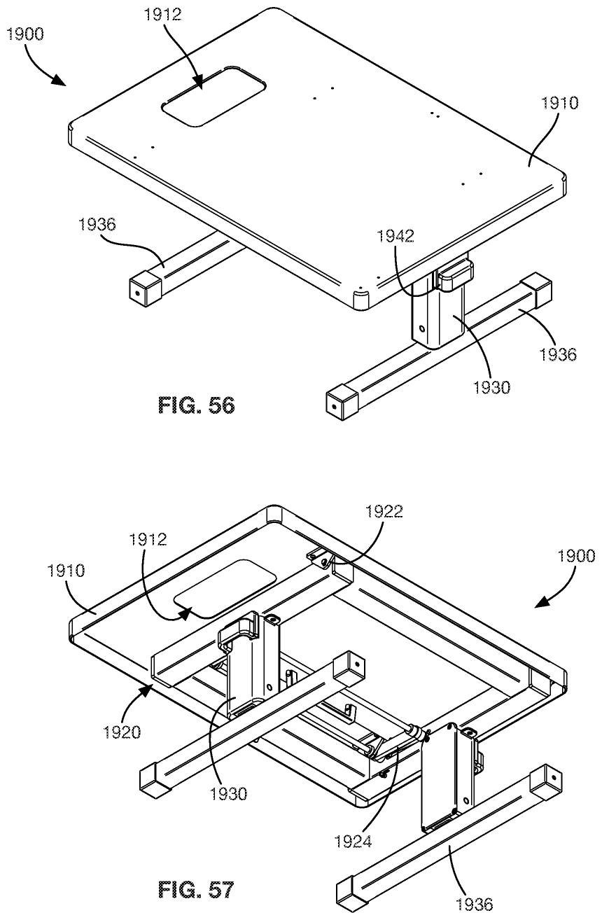

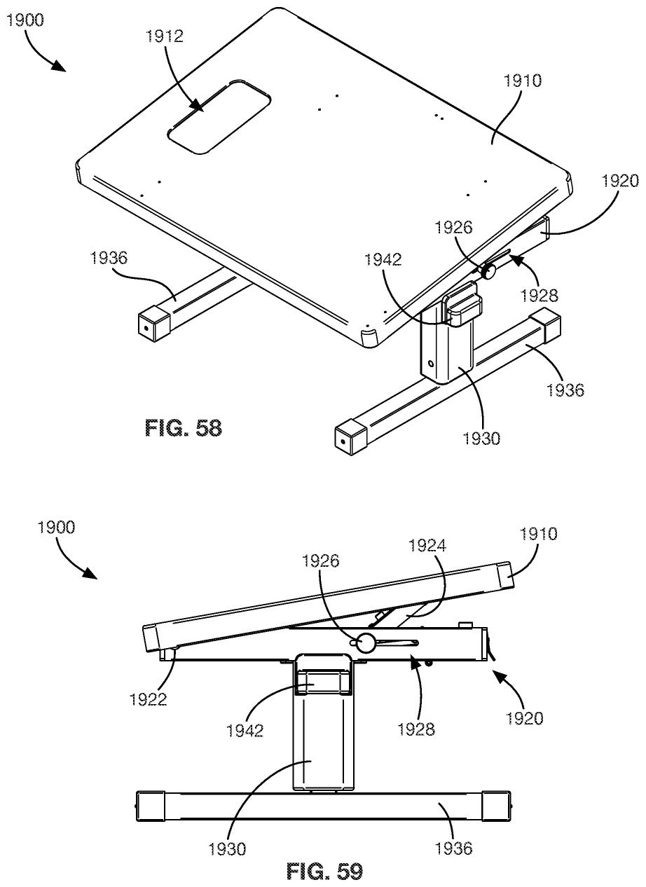

Referring now to FIGS. 56-66 an example stabilizing base 1900 for supporting a medical device/system is shown. The stabilizing base 1900 can incorporate any of the features of stabilizing bases disclosed herein and can be made from any suitable material, such as metal or plastic. The stabilizing base 1900 includes a platform 1910 that is hingeably attached to a frame 1920. Support legs 1930 extend downward from the frame 1920 to an operating table (not shown) to elevate the platform 1910 above a patient laying on the operating table. In some implementations, a rail system (not shown) attaches to the platform 1910 (or, in some implementations, is integrally formed on or as part of the platform) and can receive clamps (not shown) for securing a medical device or system, such as the delivery system or catheter assembly 114, to the rail system. The rail system can be attached to or combined with the platform 1910 in any suitable way, such as, for example, with fasteners, threaded fasteners, snaps, clamps, latches, friction fit, spring-loaded clamps, hook and loop fasteners, magnets, or the like. In some implementations, the rail system and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 1900 maintains stability by having a broad base for each of the support legs 1930 that rest on a surface such as a patient's table.

The support legs 1930 are spaced apart to allow the stabilizing base 1900 to straddle the right leg of the patient so that the rail and delivery systems can be aligned with the right inner thigh and femoral artery of the patient. The platform 1910 extends further beyond the frame 1920 and support leg 1930 on one side so that the delivery system can be repositioned to align with the left femoral artery of the patient without having to reposition the stabilizing base 1900. This is useful during an operation when a drape has been installed over the stabilizing base 1900 and repositioning the stabilizing base 1900 may not be easy or possible. An opening 1912 in the platform 1910 is provided near the support leg 1930 that is further inward from the edge of the platform 1910 so that the height adjusting mechanism of the support leg 1930, described in detail below, can be accessed by the user.

In some implementations, the platform 1910 is hingeably attached to the frame 1920 by hinges 1922 attached near one edge of the platform 1910 so that the platform 1910 can be tilted upwards to about 10 degrees, or about 15 degrees, or about 20 degrees above the frame 1920. Arranging the hinges 1922 near the front edge of the platform 1910 enables the platform 1910 to tilt upward without reducing the height between the platform 1910 and the patient. A pair of struts 1924 are hingeably attached to the platform 1910 on an upper end and are slideably attached to the frame 1920 on a lower end. A locking member or knob 1926 fixes the sliding end of the struts 1924 at a desired position within a slot 1928 to hold the platform 1910 in a tilted condition and at a desired angle relative to the frame 1920.

Referring now to FIGS. 60-63, the internal mechanism for adjusting the height of the support legs 1930 is shown in section and enlarged detail views according to some implementations. Each of the support legs 1930 includes an extendable post 1932 that is attached to the frame 1920 and a fixed post 1934 that is attached to a base or foot 1936 configured to rest on the table 110. The fixed post 1934 includes a plurality of vertically spaced apart holes 1938 for receiving latch pins 1940 of the extendable post 1932. The height of the platform 1910 can be adjusted by moving the latch pins 1940 to the unlatched position, lifting the platform 1910, and then moving the latch pins 1940 into the latched position in a new pair of holes 1938 at the desired height.

The extendable post 1932 includes handles 1942 at the upper end of each of the support legs 1930 that can be depressed to move the latch pins 1940 between the latched and unlatched positions. The handles 1942 are connected to latch rails 1944 that include slanted slots 1946 for engaging and moving protrusions 1948 of the latch pins 1940 to cause the latch pins 1940 to move laterally in outward (unlatching) or inward (latching) directions as the latch rails 1944 move vertically up and down, respectively. Both handles 1942 must be depressed by the user to unlatch and raise or lower the platform 1910.

The latch rails 1944 are in a lowered position when the handles 1942 are not engaged by the user so that the protrusions 1948 of the latch pins 1940 are moved to the upper and inward most ends of the slanted slots 1946 so that the latch pins 1940 are inserted through a pair of latch holes 1938 of the fixed post 1934, as can be seen in FIGS. 60-61. The handles 1942 can be biased to the lowered or unengaged position by handle biasing members 1950 to prohibit accidental unlatching of the latch pins 1940. That is, the latch rails 1944 are in a lowered position when the handles 1942 are not engaged by the user so that the protrusions 1948 of the latch pins 1940 are moved to the upper and inward most ends of the slanted slots 1946 so that the latch pins 1940 are inserted through the latch holes 1938 of the fixed post 1934.

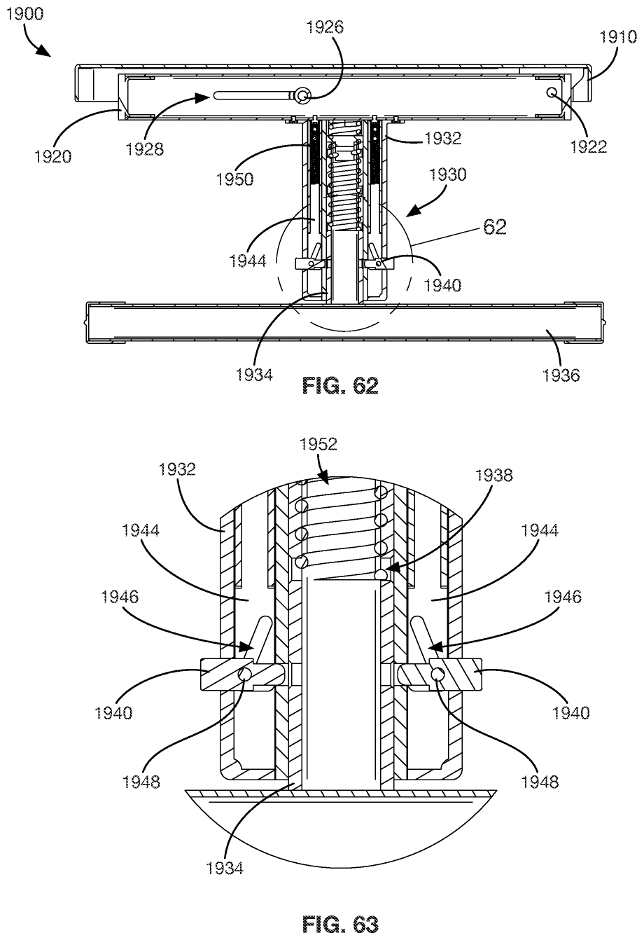

When the user depresses and lifts the handles 1942, as is shown in FIGS. 62-63, the latch rails 1944 are moved upwards so that the slanted slots 1946 engage the protrusions 1948 of the latch pins 1940 to move the latch pins 1940 outward to disengage the latch holes 1938 of the fixed post 1934 so that the extendable post 1932 and platform 1910 can be moved vertically by the user. Main biasing members 1952, such as, for example, springs shown in FIGS. 60-63, support or balance the weight of the platform 1910, the frame 1920, and any attached rail or delivery system to reduce the force required to move the platform 1910 to the desired height. In some implementations, the main biasing members 1952 are configured to provide an upward biasing force that must be overcome to push the platform 1910 downward, thereby ensuring that the platform 1910 only moves downward when intended by the user and cannot drop if the user loses their grip on the platform 1910.

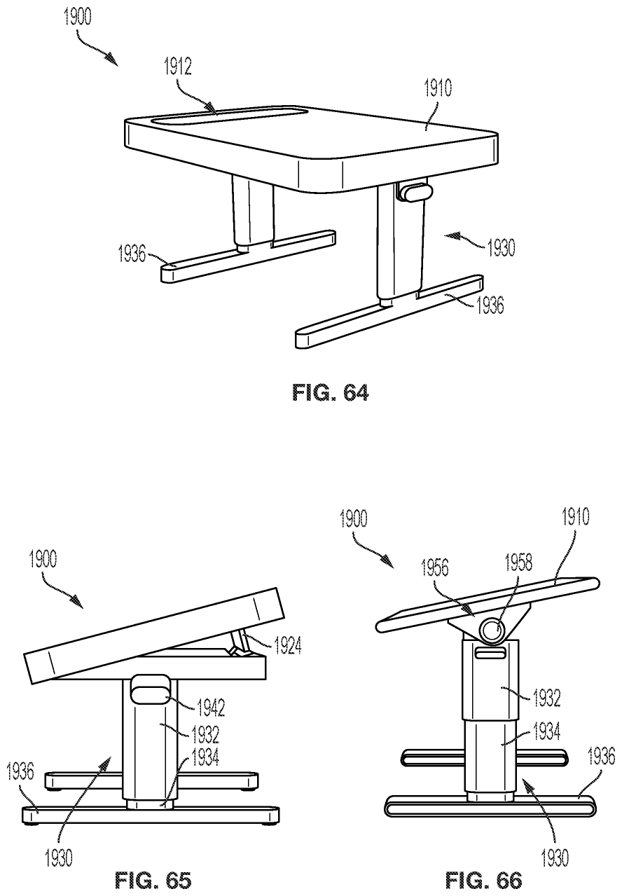

Referring now to FIGS. 64-66, the stabilizing base 1900 is shown with a different visual appearance but with the same mechanisms for raising and lower the platform 1910. The platform 1910 can be tilted at the front edge, as shown in FIG. 65, or can pivot at a central pivot point 1956 that can include a button 1958 for releasing and locking the pivot point 1956 to allow the user to adjust the tilt angle of the platform 1910 to a desired inclination. In the implementation shown in FIG. 66, a frame can be optional as the support legs 1930 can be hingeably attached directly to the platform 1910.

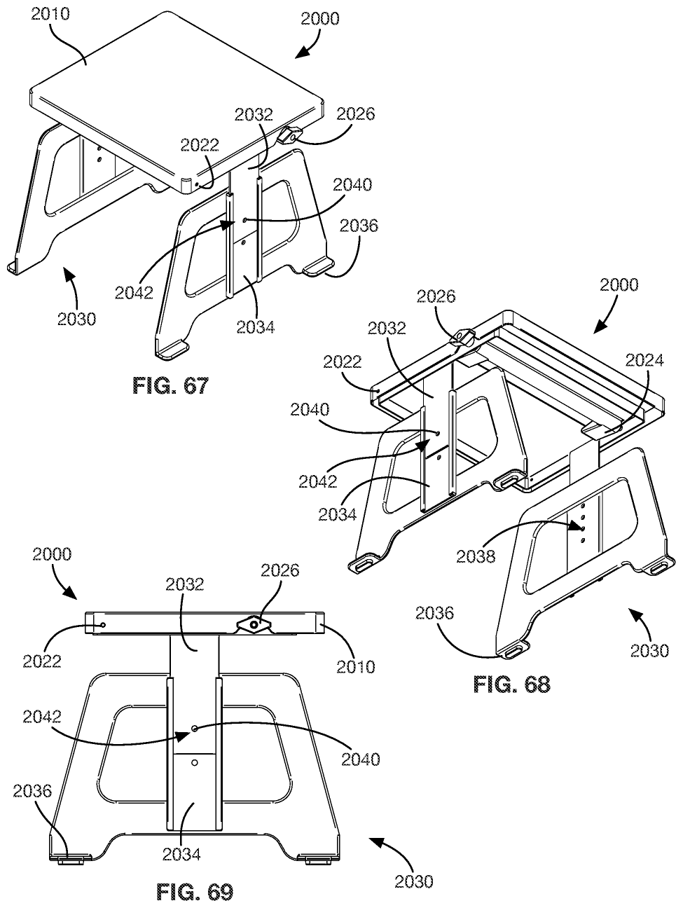

Referring now to FIGS. 67-72 an example stabilizing base 2000 for supporting a medical device/system is shown. The stabilizing base 2000 can incorporate any of the features of stabilizing bases disclosed herein and can be made from any suitable material, such as metal, plastic, etc. The stabilizing base 2000 includes a platform 2010 that is hingeably attached to a frame 2020. Support legs 2030 extend downward from the frame 2020 to an operating table (not shown) to elevate the platform 2010 above a patient laying on the operating table. In some implementations, a rail system (not shown) attaches to the platform 2010 (or, in some implementations, can be integrally formed on or as part of the platform) and can receive clamps (not shown) for securing a medical device or system, such as the delivery system or catheter assembly (not shown), to the rail system. In some implementations, the rail system can be attached to the platform 2010 in any suitable way, such as, for example, with fasteners, threaded fasteners, snaps, clamps, latches, friction fit, spring-loaded clamps, hook and loop fasteners, magnets, or the like. In some implementations, the rail system and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 2000 maintains stability by having a broad base for each of the support legs 2030 that rest on a surface such as a patient's table. The support legs 2030 are spaced apart to allow the stabilizing base 2000 to straddle the right leg of the patient so that the rail and delivery systems can be aligned with the right inner thigh and femoral artery of the patient.

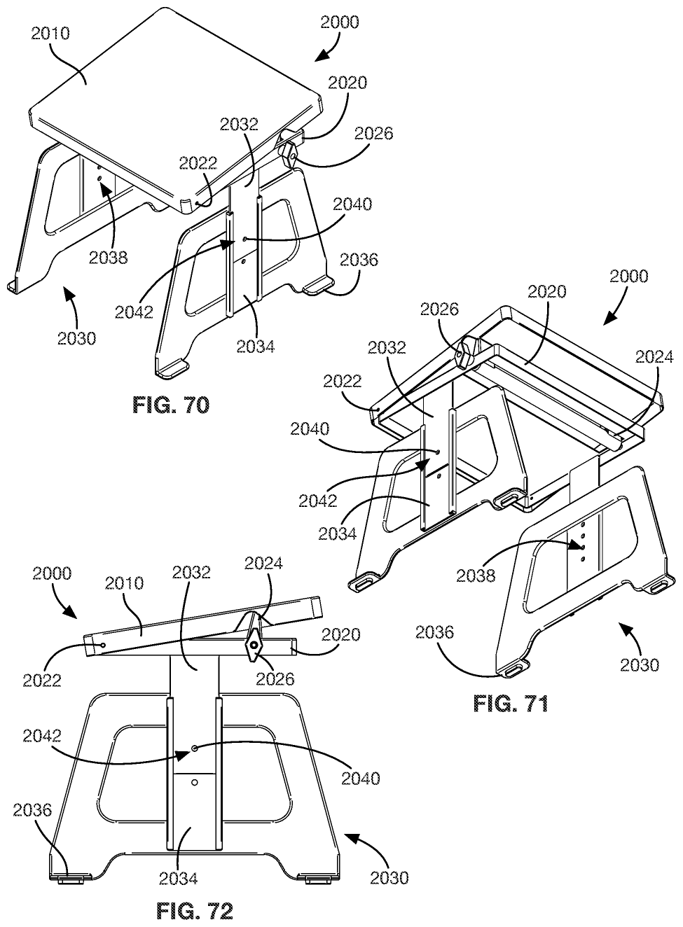

In some implementations, the platform 2010 is hingeably attached to the frame 2020 by hinges 2022 attached near one edge of the platform 2010 so that the platform 2010 can be tilted upwards from the flat position shown in FIGS. 67-69 to about 10 degrees, or about 15 degrees, or about 20 degrees above the frame 2020 as can be seen in FIGS. 70-72. Arranging the hinges 2022 near the front edge of the platform 2010 enables the platform 2010 to tilt upward without reducing the height between the platform 2010 and the patient. A pair of cams 2024 can be rotated from a stowed to a deployed position by turning a knob 2026 on the side of the frame 2020. The cams 2024 engage the platform 2010 to lift the platform 2010 from a flat to a tilted position. In some implementations, the cams 2024 can be locked in place in an intermediate position between the stowed and deployed position to provide additional granularity in the tilt position of the platform 2010.

Each of the support legs 2030 includes an extendable post 2032 that is attached to the frame 2020 and a fixed post 2034 that is attached to a base or feet 2036 configured to rest on the table 110. The fixed post 2034 includes a plurality of vertically spaced apart holes 2038 for receiving a fastener 2040 that is inserted through the holes 2038 of the fixed post 2034 and a positioning hole 2042 of the extendable post 2032. The height of the platform 2010 can be adjusted by removing the fasteners 2040 in each support leg 2030, repositioning the moveable posts 2032, and re-installing the fasteners 2040 through a different hole 2038.

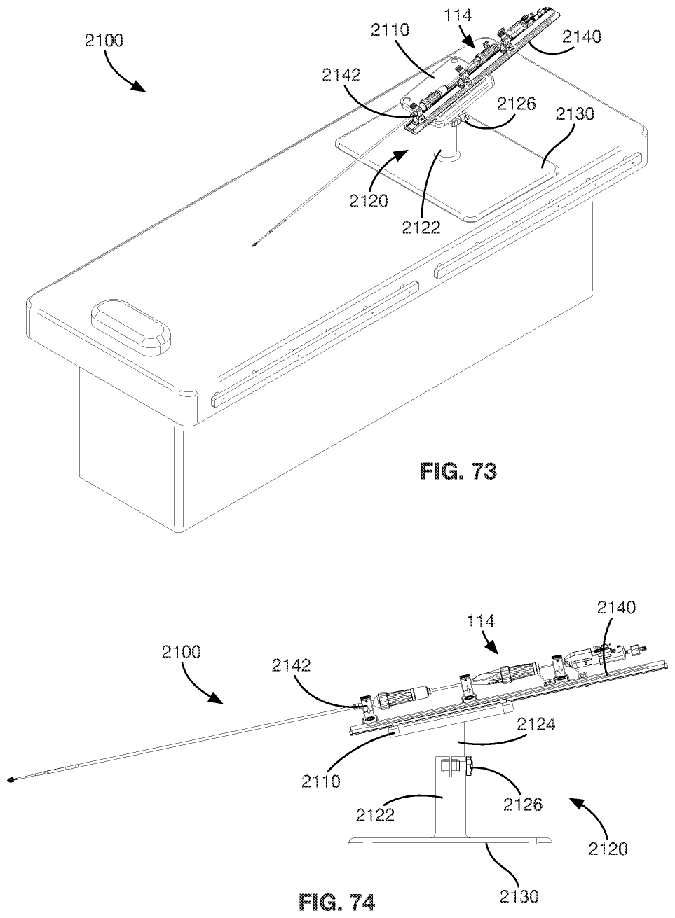

Referring now to FIGS. 73-74 an example stabilizing base 2100 for supporting a medical device/system is shown. The stabilizing base 2100 can incorporate any of the features of stabilizing bases disclosed herein and can be made from any suitable material, such as metal, plastic, etc. The stabilizing base 2100 includes a platform 2110, a post 2120, and abase plate 2130. The platform 2110 is supported above the base plate 2130 by the post 2120 that extends vertically between the platform 2110 and the base plate 2130. In some implementations, a rail system 2140 attaches to the platform 2110 (or, in some implementations, is integrally formed on or as part of the platform) and can receive clamps 2142 for securing a medical device or system, such as the delivery system or catheter assembly 114, to the rail system 2140. The rail system 2140 can be attached to the platform 2110 in any suitable way, such as, for example, with fasteners, threaded fasteners, snaps, clamps, latches, friction fit, spring-loaded clamps, hook and loop fasteners, magnets, or the like. In some implementations, the rail system 2140 and/or the clamps 2142 are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 2100 maintains stability by having a broad base plate 2130 to rest on a surface such as a patient's table.

The base plate 2130 can be a substantially flat plate of any suitable shape and dimension to provide counterbalance and support to the medical device/system secured to the stabilizing base 2100. The base plate can be square, round, hexagonal, and the like. The base plate 2130 can be solid or can have a void space within the center of the plate. In some implementations, the base plate 2130 can be a solid rectangular plate. The base plate 2130 can be placed flush against the surface of a table 110 as shown in FIG. 1. A patient's legs can rest atop the base plate 2130 such that the post 2120 extends vertically upwards in between the patient's legs. The position of the delivery system/catheter assembly 114 relative to the patient can be optimized by adjusting the height of the platform 2110.

The platform 2110 to which the rail system 2140 and/or delivery system 114 are attached is supported vertically above the base plate 2130 by the post 2120. The platform 2110 can be attached to the post 2120 by any suitable means, such as welding, fasteners, and/or an adhesive, or the like. The platform 2110 is attached to the post 2120 at a fixed angle relative to the base plate 2130 that is a desirable angle for deploying the implantable device.

In some implementations, the post 2120 includes a fixed portion 2122 and a moveable portion 2124 and can be any suitable shape, such as square, circular, or oblong. The fixed portion 2122 of the post 2120 is fixedly attached to the base plate 2130 by any suitable means, such as, for example, with welding, adhesives, fasteners, a clamp, or the like. The moveable portion 2124 slides vertically in a telescoping manner within the fixed portion 2122 to enable vertical translation of the platform 2110 to a desired height. When the platform 2110 has been raised or lowered to a desired height, a clamp 2126 on the fixed portion 2122 of the post 2120 is tightened to squeeze the end of the fixed portion 2122 against the moveable portion 2124 to prohibit further movement of the moveable portion 2124 during use of the stabilizing base 2100. An optional mechanical or gas spring mechanism (not shown) within the post 2120 can be used to balance out the weight of the delivery system 114 and the platform 2110 so that the height of the platform 2110 can be more easily adjusted by the user.

The rail system 2140 can be configured to be entirely or partially retained by features of the platform 2110 of the stabilizing base 2100 so that when the stabilizing base 2100 is covered by a sterile barrier (not shown), such as a drape, the rail system 2140 can still be attached to the stabilizing base 2100. The barriers 112 illustrated by FIG. 1 can be placed between the stabilizing base 2100 and the rail system 2140. The rail system 2140 can be attached to the stabilizing base 2100 without attachment means, such as fasteners, that would pierce or puncture the sterile barrier arranged between the platform 2110 and the rail system 2140. In some implementations, the rail system 2140 can be attached to the stabilizing base 2100 using magnetic plates or snap connections that do not puncture a sterile barrier. In some implementations, the horizontal position of the rail system 2140 can be adjusted relative to the platform 2110, such as by sliding the rail system 2140 forwards and backwards in relation to the platform 2110.

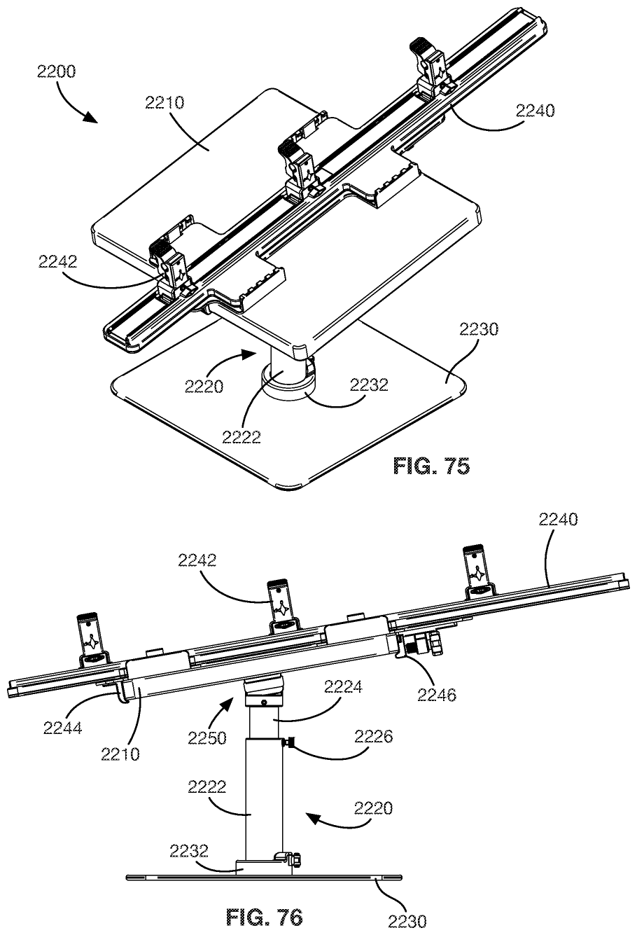

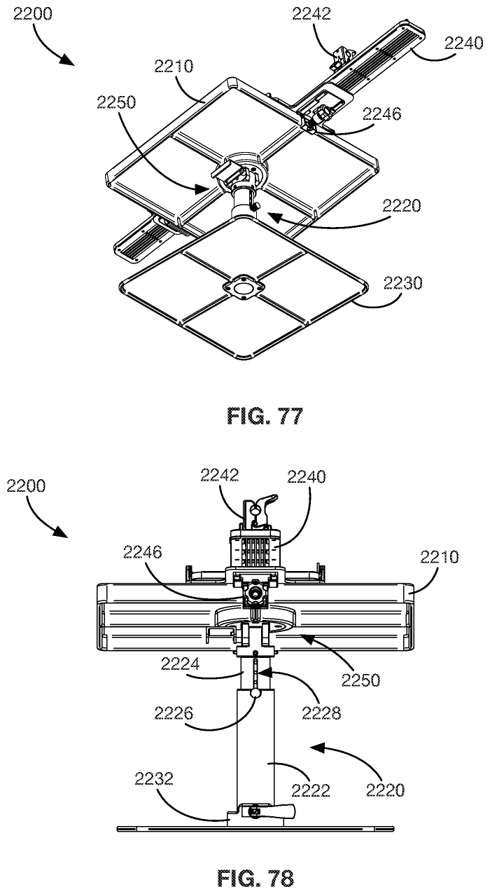

Referring now to FIGS. 75-84 an example stabilizing base 2200 for supporting a medical device/system is shown. The stabilizing base 2200 can incorporate any of the features of stabilizing bases disclosed herein and can be made from any suitable material, such as metal, plastic, etc. The stabilizing base 2200 includes a platform 2210, a post 2220, and a base plate 2230. The platform 2210 is supported above the base plate 2230 by the post 2220 that extends vertically between the platform 2210 and the base plate 2230. In some implementations, a rail system 2240 attaches to the platform 2210 (or, in some implementations, is integrally formed on or as part of the platform) and can receive clamps 2242 for securing a medical device or system, such as the delivery system or catheter assembly (not shown), to the rail system 2240. The rail system 2240 can be attached to the platform 2210 in any suitable way, such as, for example, with fasteners, threaded fasteners, snaps, clamps, latches, friction fit, spring-loaded clamps, hook and loop fasteners, magnets, or the like. In some implementations, the rail system 2240 and/or the clamps 2242 are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 2200 maintains stability by having a broad base plate 2230 to rest on a surface such as a patient's table.

The base plate 2230 can be a substantially flat plate of any suitable shape and dimension to provide counterbalance and support to the medical device/system secured to the stabilizing base 2200. The base plate can be square, round, hexagonal, and the like. The base plate 2230 can be solid or can have a void space within the center of the plate. In some implementations, the base plate 2230 can be a solid rectangular plate. The base plate 2230 can be placed flush against the surface of a table 110 as shown in FIG. 1. A patient's legs can rest atop the base plate 2230 such that the post 2220 extends vertically upwards in between the patient's legs. The position of the delivery system/catheter assembly relative to the patient can be optimized by adjusting the height of the platform 2210.

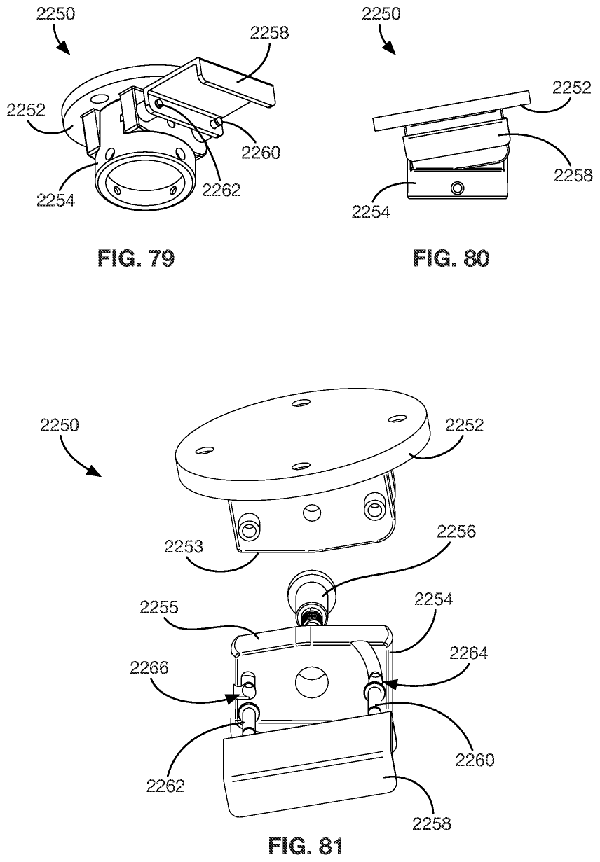

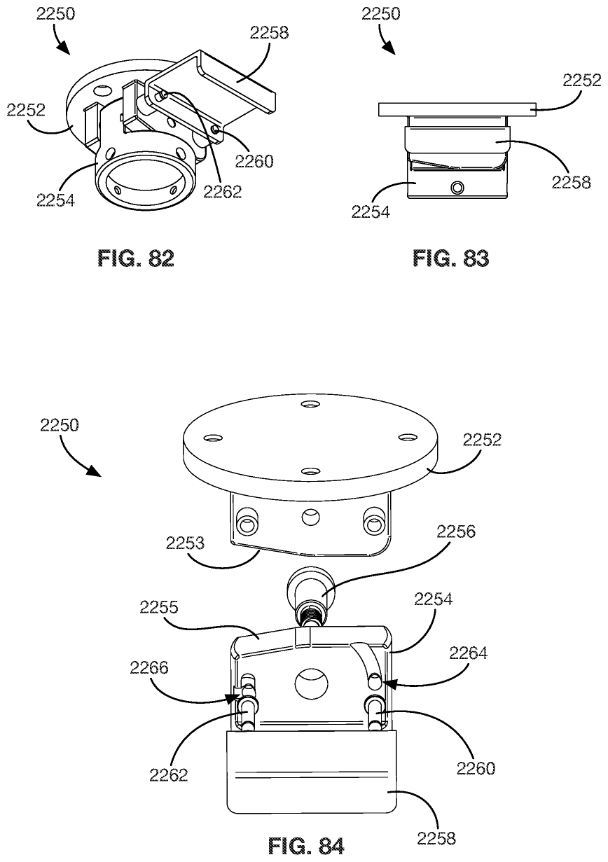

The platform 2210 to which the rail system 2240 and/or delivery system 114 are attached is supported vertically above the base plate 2230 by the post 2220. In some implementations, the platform 2210 is hingeably attached to the moveable portion 2224 of the post 2220 so that the user can pivot the platform 2210 between flat and tilted positions. A hinge assembly 2250 that connects the platform 2210 to the post 2220 is shown in the tilted position in FIGS. 79-81 and in the flat position in FIGS. 82-84. The hinge assembly 2250 includes a pivoting bracket 2252 that is pivotably connected to a stationary bracket 2254 by a pivot 2256. The pivoting bracket 2252 is connected to the platform 2210 and the stationary bracket 2254 is connected to the moveable portion 2224 of the post 2220. The pivot 2256 can be any suitable component or mechanism that allows the pivoting bracket 2252 to tilt relative to the stationary bracket 2254, such as, for example, the shoulder screw shown in FIGS. 81 and 84, a shaft, a pin, a hinge, or the like. The pivoting bracket 2252 and the stationary bracket 2254 can include angled surfaces 2253, 2255, respectively, that provide clearance for the pivoting bracket 2252 to rotate. The angled surfaces 2253, 2255 can also function to prohibit further tilting of the platform 2210 beyond the predetermined tilt angle.

The pivoting bracket 2252 can be locked in the tilted or flat position and can be pivoted by pulling on a release lever 2258 to unlock the pivoting bracket 2252 so that the platform 2210 can be tilted. A first locking pin 2260 and a second locking pin 2262 extend through the pivoting bracket 2252 into a first locking hole 2264 and a second locking hole 2266, respectively, to lock the position of the pivoting bracket 2252 relative to the stationary bracket 2254. Pulling on the release lever 2258 disengages the first and second locking pins 2260, 2262 from the first and second locking holes 2264, 2266. The first locking hole 2264 is arranged so that the first locking pin 2260 aligns with and engages the first locking hole 2264 when the platform 2210 is in the flat position. The second locking hole 2266 is arranged lower than the first locking hole 2264 so that the second locking pin 2262 aligns with and engages the second locking hole 2266 when the platform 2210 is in the tilted position. Because of the different heights of the locking holes 2264, 2266, only one of the two locking pins 2260, 2262 is engaged at a time.

In some implementations, the post 2220 includes a fixed portion 2222 and a moveable portion 2224 and can be any suitable shape, such as square, circular, or oblong. The fixed portion 2222 of the post 2220 is removably attached to the base plate 2230 by a clamp 2232 that is fixedly attached to the base plate 2230 by any suitable means. The clamp 2232 is opened to receive the fixed portion 2222 of the post 2220 and is then tightened to secure the post 2220 to the base plate 2230. The moveable portion 2224 of the post 2220 slides vertically in a telescoping manner within the fixed portion 2222 to enable vertical translation of the platform 2210 to a desired height. A locking pin 2226 is retracted to allow the moveable portion 2224 to move and the locking pin 2226 is inserted into a locking hole 2228 of the moveable portion 2224 to retain the moveable portion 2224 at the desired height. The locking pin 2226 can be biased toward the locking direction to prohibit accidental disengagement of the locking pin 2226 that may allow the movable portion 2224 to drop. An optional mechanical or gas spring mechanism (not shown) within the post 2220 can be used to balance out the weight of the delivery system 114 and the platform 2210 so that the height of the platform 2210 can be more easily adjusted by the user and to prohibit unintentional lowering of the platform 2210.

In some implementations, the rail system 2240 can be configured to be entirely or partially retained by features of the platform 2210 of the stabilizing base 2200 so that when the stabilizing base 2200 is covered by a sterile barrier (not shown), such as a drape, the rail system 2240 can still be attached to the stabilizing base 2200. The barrier 112 illustrated by FIG. 1 can be placed between the stabilizing base 2200 and the rail system 2240. The rail system 2240 can be attached to the stabilizing base 2200 without attachment means, such as fasteners, that would pierce or puncture the sterile barrier arranged between the platform 2210 and the rail system 2240. For example, the rail system 2240 can include a fixed jaw 2244 and a moveable jaw 2246 for gripping the platform 2210. The moveable jaw 2246 can be retracted to allow the rail system 2240 to be attached to the platform 2210 and then tightened against the platform 2210 via any suitable clamping mechanism, such as the illustrated screw clamp. In some implementations, the rail system 2240 can be attached to the stabilizing base 2200 using magnetic plates or snap connections that do not puncture a sterile barrier. In some implementations, the horizontal position of rail system 2240 can be adjusted relative to the platform 2210, such as by sliding the rail system 2240 laterally in relation to the platform 2210.

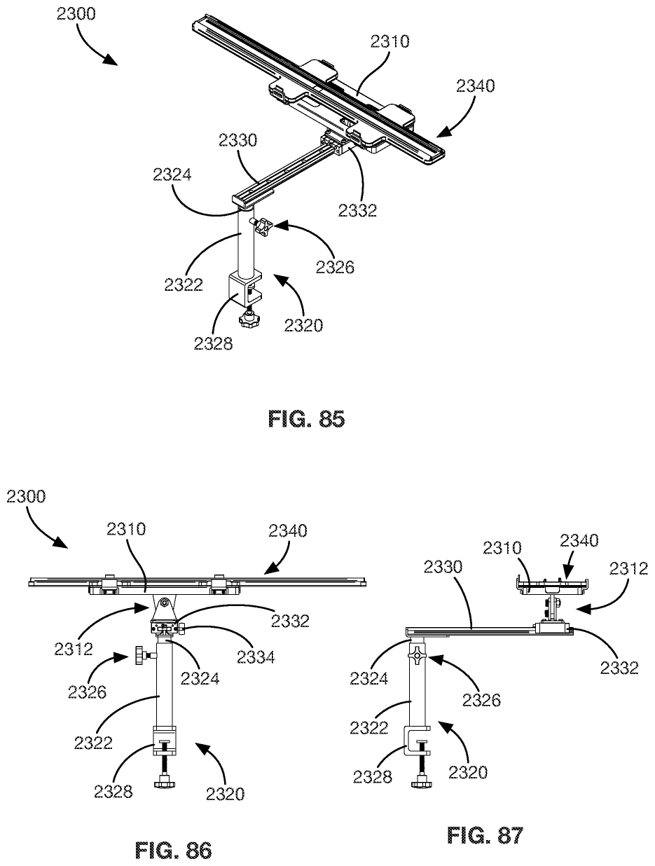

Referring now to FIGS. 85-87 an example stabilizing base 2300 for supporting a medical device/system is shown. The stabilizing base 2300 can incorporate any of the features of stabilizing bases disclosed herein and can be made from any suitable material, such as metal or plastic. The stabilizing base 2300 is configured to attach to a side rail of an operating table 110 and includes a platform 2310, a vertical post 2320, and a horizontal arm 2330. The platform 2310 is supported above the table 110 by the post 2320 that extends vertically between the horizontal arm 2330 and a side rail of the table 110.

In some implementations, a rail system 2340 attaches to the platform 2310 (or, in some implementations, is integrally formed on or as part of the platform) and can receive clamps (not shown) for securing a medical device or system, such as the delivery system or catheter assembly 114, to the rail system 2340. The clamps are not shown in FIGS. 85-87 to simplify the drawings and can be the clamps of any of the implementations described herein. The rail system 2340 can be attached to the platform 2310 in any suitable way, such as, for example, with fasteners, threaded fasteners, snaps, clamps, latches, friction fit, spring-loaded clamps, hook and loop fasteners, magnets, or the like. In some implementations, the rail system 2340 and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 2300 maintains stability by virtue of the rigidity of the vertical post 2320 and the horizontal arms 2330 that are attached to the operating table 110.

The platform 2310 to which the rail system 2340 and delivery system 114 are attached is supported vertically above the table 110 by the vertical post 2320 and the horizontal arm 2330. In some implementations, the platform 2310 is hingeably attached via a hinge 2312 to a carriage 2332 that slides along the horizontal arm 2330. The platform 2310 can be pitched fore and aft via the hinge 2312 to tilt the platform 2310 and attached rail system 2340 and delivery system 114. The carriage 2332 allows the platform 2310 to slide laterally along the horizontal arm 2330 to laterally position the delivery system 114 to align with, for example, the patient's right femoral artery. The hinge 2312 and the carriage 2332 can include locking devices (not shown), such as a set screw or the like, for locking the tilted condition of the platform 2310 relative to the top of the operating table 110. A clamp 2334 can be used to lock the lateral position of the carriage 2332 along the horizontal rail 2330.

The vertical post 2320 includes a fixed portion 2322 and a moveable portion 2324 and can be any suitable shape, such as square, circular, or oblong. The moveable portion 2324 slides vertically in a telescoping manner within the fixed portion 2322 to enable vertical translation of the horizontal arm 2330 and the platform 2310 to a desired height above the table 110. When the platform 2310 has been raised or lowered to a desired height, a clamp 2326 on the fixed portion 2322 of the post 2320 is tightened to engage and prohibit further movement of the moveable portion 2324 during use of the stabilizing base 2300. An optional mechanical or gas spring mechanism (not shown) within the post 2320 can be used to balance out the weight of the delivery system 114, the platform 2310, and the horizontal arm 2330 so that the height of the platform 2310 can be more easily adjusted by the user. The fixed portion 2322 of the vertical post 2320 is removably attached to a side rail of the operating table 110 with a clamp 2328 to enable the stabilizing base 2300 to be moved along the length of the table 110 by the user.

In some implementations, the rail system 2340 can be configured to be entirely or partially retained by features of the platform 2310 of the stabilizing base 2300 so that when the stabilizing base 2300 is covered by a sterile barrier (not shown), such as a drape, the rail system 2340 can still be attached to the stabilizing base 2300. The illustrated barriers 112 can be placed between the stabilizing base 2300 and the rail system 2340. The rail system 2340 can be attached to the stabilizing base 2300 without attachment means, such as fasteners, that would pierce or puncture the sterile barrier arranged between the platform 2310 and the rail system 2340. In some implementations, the rail system 2340 can be attached to the stabilizing base 2300 using magnetic plates or snap connections that do not puncture a sterile barrier. In some implementations, the horizontal position of rail system 2340 can be adjusted relative to the platform 2310, such as by sliding the rail system 2340 in relation to the platform 2310.

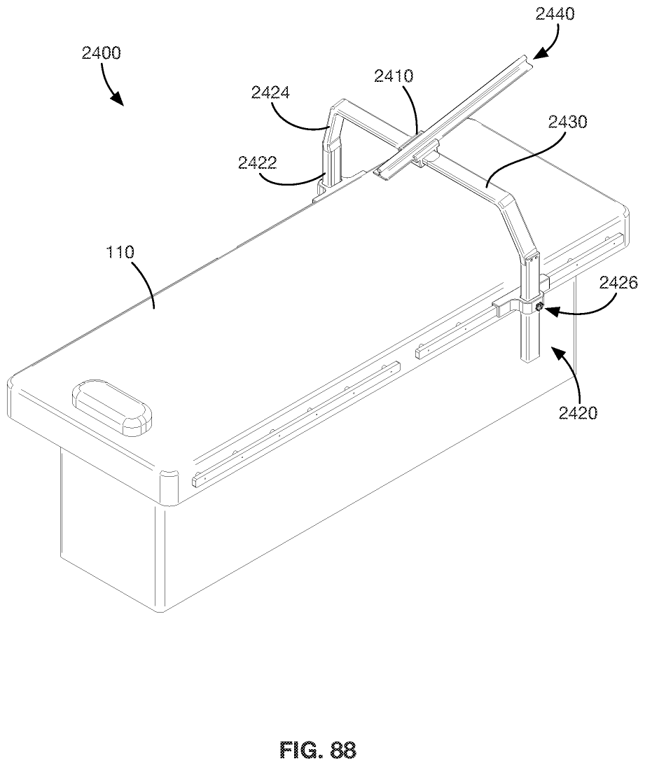

Referring now to FIG. 88 an example stabilizing base 2400 for supporting a medical device/system is shown. The stabilizing base 2400 can incorporate any of the features of stabilizing bases disclosed herein and can be made from any suitable material, such as metal or plastic. The stabilizing base 2400 is similar to the implementation shown in FIGS. 35-36, however, the stabilizing base 2400 is configured to attach to both side rails of an operating table 110. The stabilizing base 2400 includes a carriage 2410, two support posts 2420, and a horizontal arm 2430. The carriage 2410 is supported above the table 110 by the posts 2420 that extend from the side rails of the table 110 to the horizontal arm 2430.

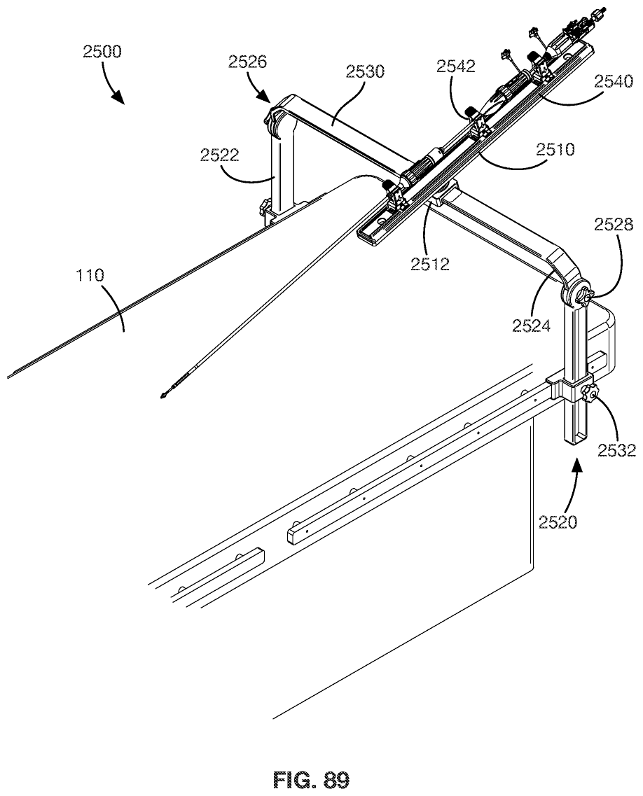

In some implementations, a rail system 2440 slideably attaches to the carriage 2410 and can receive clamps (See FIG. 89) for securing a medical device or system, such as the delivery system or catheter assembly 114, to the rail system 2440. The clamps are not shown in FIG. 88 to simplify the drawings and can be the clamps of any of the implementations described herein. In some implementations, the rail system 2440 and/or the clamps are the same as those disclosed by U.S. Provisional Patent Application Ser. No. 63/073,392, filed on Sep. 1, 2020, and/or PCT Application No. PCT/US2021/048333, filed on Aug. 31, 2021, which are incorporated herein by reference in their entireties. The stabilizing base 2400 maintains stability by virtue of the rigidity of the support posts 2420 and the horizontal arm 2430 that are attached to the side rails of the operating table 110.

The carriage 2410 to which the rail system 2440 and delivery system 114 are attached is supported vertically above the table 110 by the support posts 2420 and the horizontal arm 2430. In some implementations, the carriage 2410 slides along the horizontal arm 2430 to laterally position the delivery system 114 to align with, for example, the patient's right femoral artery. The carriage 2410 can include a locking device (not shown), such as a set screw or the like, for locking the lateral position of the carriage 2410 along the horizontal rail 2430. The carriage 2410 is tilted or pitched at a fixed angle based on a tilt or pitch angle of the horizontal rail 2430 relative to the top surface of the operating table 110.

In some implementations, the support posts 2420 each include a vertical portion 2422 and a slanted or angled portion 2424 and can be any suitable shape, such as square, circular, or oblong. The vertical portion 2422 and the slanted portion 2424 can be integrally formed from one piece or can be connected in any suitable way, such as, for example, with fasteners, welding, adhesives, mortice and tenon connections, pinned connections, or the like. The vertical portions 2422 of the support posts are slideably attached to clamps 2426. The clamps 2426 can be attached to or part of mounts that attached to the side rails of the operating table 110. The clamps 2426 can be loosened to enable vertical translation of the horizontal arm 2430 and the carriage 2410 to a desired height above the table 110. When the carriage 2410 has been raised or lowered to a desired height, the clamps 2426 are tightened to engage and prohibit further movement of the support posts 2420 during use of the stabilizing base 2400. The mounts and included clamps 2428 can be slideably attached to the side rails of the operating table 110 to enable the stabilizing base 2400 to be moved along the length of the table 110 by the user.

In some implementations, the rail system 2440 can be configured to be entirely or partially retained by features of the carriage 2410 of the stabilizing base 2400 so that when the stabilizing base 2400 is covered by a sterile barrier (not shown), such as a drape, the rail system 2440 can still be attached to the stabilizing base 2400. The barrier 112 illustrated by FIG. 1 can be placed between the stabilizing base 2400 and the rail system 2440. The rail system 2440 can be attached to the stabilizing base 2400 without attachment means, such as fasteners, that would pierce or puncture the sterile barrier arranged between the carriage 2410 and the rail system 2440. In some implementations, the rail system 2440 can be attached to the stabilizing base 2400 using magnetic plates or snap connections that do not puncture a sterile barrier. In some implementations, the horizontal position of rail system 2440 can be adjusted relative to the platform 2410, such as by sliding the rail system 2440 forwards and backwards in relation to the platform 2410.

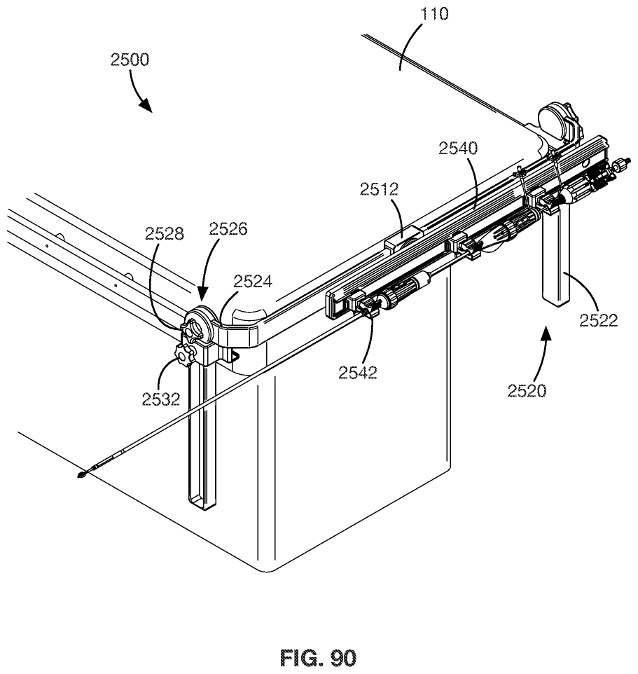

Referring now to FIGS. 89-90 an example stabilizing base 2500 for supporting a medical device/system is shown. The stabilizing base 2500 can incorporate any of the features of stabilizing bases disclosed herein and can be made from any suitable material, such as metal or plastic. Like the stabilizing base 2400 described above, the stabilizing base 2500 is configured to attach to both side rails of an operating table 110. The stabilizing base 2500 includes a platform 2510, two support posts 2520, and a horizontal arm 2530. The platform 2510 is supported above the table 110 by the posts 2520 that extend from the side rails of the table 110 to the horizontal arm 2530.

In some implementations, a rail system 2540 slideably attaches to the platform 2510 and can receive clamps 2542 for securing a medical device or system, such as the delivery system or catheter assembly 114, to the rail system 2540. In some implementations, the rail system 2540 and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. The stabilizing base 2500 maintains stability by virtue of the rigidity of the support posts 2520 and the horizontal arm 2530 that are attached to the side rails of the operating table 110.

The platform 2510 to which the rail system 2540 and delivery system 114 are attached is supported vertically above the table 110 by the support posts 2520 and the horizontal arm 2530. In some implementations, the platform 2510 is rotatably attached to a carriage 2512 that is slideably attached to the horizontal arm 2530 and can slide along the horizontal arm 2530 to laterally position the delivery system 114 to align with, for example, the patient's right femoral artery. The carriage 2512 can include a locking device (not shown), such as a set screw or the like, for locking the lateral position of the carriage 2512 and the platform 2510 along the horizontal rail 2530.

The support posts 2520 can be any suitable shape, such as square, circular, or oblong and each support post 2520 includes a vertical portion 2522 and a moveable or pivoting portion 2524 that is attached to the fixed portion 2522 with a hinge 2526. In some implementations, the hinge 2526 includes a locking member 2528 that can be actuated to lock the position of the hinge 2526. When the locking member 2528 is disengaged, the moveable portion 2524 can be pivoted relative to the fixed portion 2522 to change the orientation of the horizontal arm 2530 and, consequently, the platform 2510 and attached rail system 2540. That is, the pitch or tilt of the delivery system 114 relative to the top surface of the table 110 can be altered by pivoting the moveable portion 2524 of the support posts 2520.

In some implementations, the vertical portions 2522 of the support posts are slideably attached to clamps 2532. The clamps 2532 can be attached to or part of mounts that are coupled to the side rails of the operating table 110. The clamps 2532 can be loosened to enable vertical translation of the horizontal arm 2530 and the platform 2510 to a desired height above the table 110. When the platform 2510 has been raised or lowered to a desired height, the clamps 2532 are tightened to engage and prohibit further movement of the support posts 2520 during use of the stabilizing base 2500. The mounts and attached clamps 2532 can be slideably attached to the side rails of the operating table 110 to enable the stabilizing base 2500 to be moved along the length of the table 110 by the user.

In some implementations, the rail system 2540 can be configured to be entirely or partially retained by features of the carriage 2510 of the stabilizing base 2500 so that when the stabilizing base 2500 is covered by a sterile barrier (not shown), such as a drape, the rail system 2540 can still be attached to the stabilizing base 2500. The barrier 112 illustrated by FIG. 1 can be placed between the stabilizing base 2500 and the rail system 2540. The rail system 2540 can be attached to the stabilizing base 2500 without attachment means, such as fasteners, that would pierce or puncture the sterile barrier arranged between the platform 2510 and the rail system 2540. In some implementations, the rail system 2540 can be attached to the stabilizing base 2500 using magnetic plates or snap connections that do not puncture a sterile barrier. In some implementations, the position of rail system 2540 can be adjusted relative to the platform 2510, such as by sliding the rail system 2540 forwards and backwards in relation to the platform 2510.

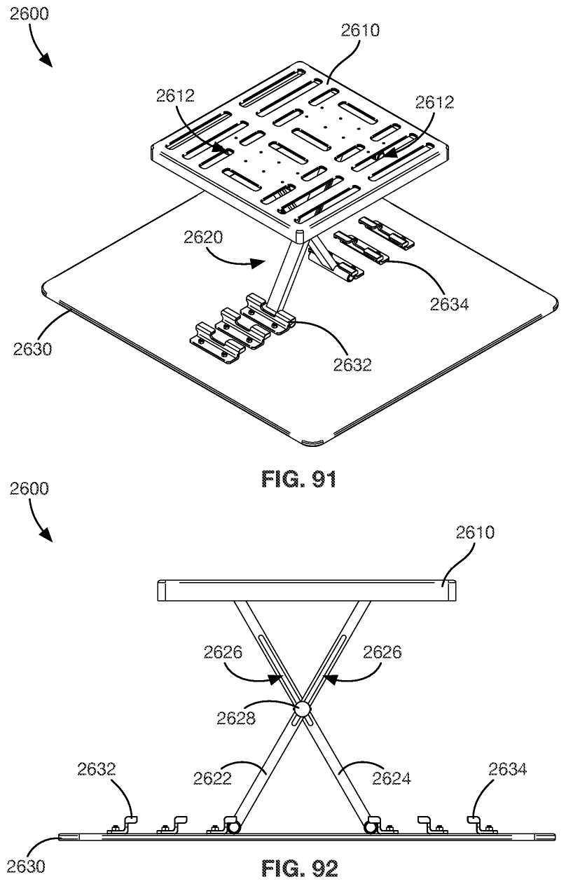

Referring now to FIGS. 91-94 an example stabilizing base 2600 for supporting a medical device/system is shown. The stabilizing base 2600 can incorporate any of the features of stabilizing bases disclosed herein and can be made from any suitable material, such as metal or plastic. The stabilizing base 2600 can incorporate any of the features of stabilizing bases disclosed herein and can be made from any suitable material, such as metal or plastic. The stabilizing base 2600 includes a platform 2610, a support frame 2620, and a base plate 2630. The platform 2610 is supported above the base plate 2630 by the support frame 2620 that extend vertically between the platform 2610 and the base plate 2630. In some implementations, a rail system (not shown) attaches to the platform 2610 (or, in some implementations, is integrally formed on or as part of the platform) and can receive clamps (not shown) for securing a medical device or system, such as the delivery system or catheter assembly, to the rail system. The rail system can be attached to the platform 2610 in any suitable way, such as, for example, with fasteners, threaded fasteners, snaps, clamps, latches, friction fit, spring-loaded clamps, hook and loop fasteners, magnets, or the like. In some implementations, the rail system and/or the clamps are the same as those disclosed by U.S. Provisional Patent Application Ser. No. 63/073,392, filed on September 1, and/or PCT Application No. PCT/US2021/048333, filed on Aug. 31, 2021, which are incorporated herein by reference in their entireties. The stabilizing base 2600 maintains stability by having a broad base plate 2630 to rest on a surface such as a patient's table.

The base plate 2630 can be a substantially flat plate of any suitable shape and dimension to provide counterbalance and support to the medical device/system secured to the stabilizing base 2600. The base plate can be square, round, hexagonal, and the like. The base plate 2630 can be solid or can have a void space within the center of the plate. In some implementations, the base plate 2630 can be a solid rectangular plate. The base plate 2630 can be placed flush against the surface of a table 110 as shown in FIG. 1. A patient's legs can rest atop the base plate 2630 such that the support frame 2620 extend vertically upwards in between the patient's legs. The position of the delivery system/catheter assembly relative to the patient can be optimized by adjusting the height of the platform 2610.

The platform 2610 to which the rail system and delivery system are attached is supported vertically above the base plate 2630 by the support frame 2620. In some implementations, the support frame 2620 includes a first support strut 2622 and a second support strut 2624. The first and second support struts 2622, 2624 are pivotably attached to the platform 2610 by upper brackets 2612. The first support strut 2622 is removably and pivotably attached to the base plate 2630 at one of a set of first lower brackets 2632. The second support strut 2622 is removably and pivotably attached to the base plate 2630 at one of a set of second lower brackets 2634. Each of the first and second support struts 2622, 2624 includes a slot 2626 through which a pivot connection 2628 extends. The moveable pivot connection 2628 can be any suitable connection-such as, for example, a through bolt with a knob on one end, as shown in FIG. 94 between the two slots 2626 of the first and second support struts 2622, 2624 that enables the position of the pivot connection 2628 to be adjusted and locked in place at any location along the length of the slots 2626.

In some implementations, the height and tilt angle of the platform 2610 can be changed by alternating the angle of one or both of the first and second support struts 2622, 2624 and the lower bracket 2632, 2634 to which the first support strut 2622 or the second support strut 2624 is attached. The first and second support struts 2622, 2624 can be moved by loosening the moveable pivot connection 2628 to allow the moveable pivot connection to slide along the slots 2626 in each of the first and second support struts 2622, 2624. When the desired orientation and height of the platform 2610 is achieved, the pivot connection 2628 is tightened to prohibit further movement of the pivot connection 2628 and the first and second support struts 2622, 2624.

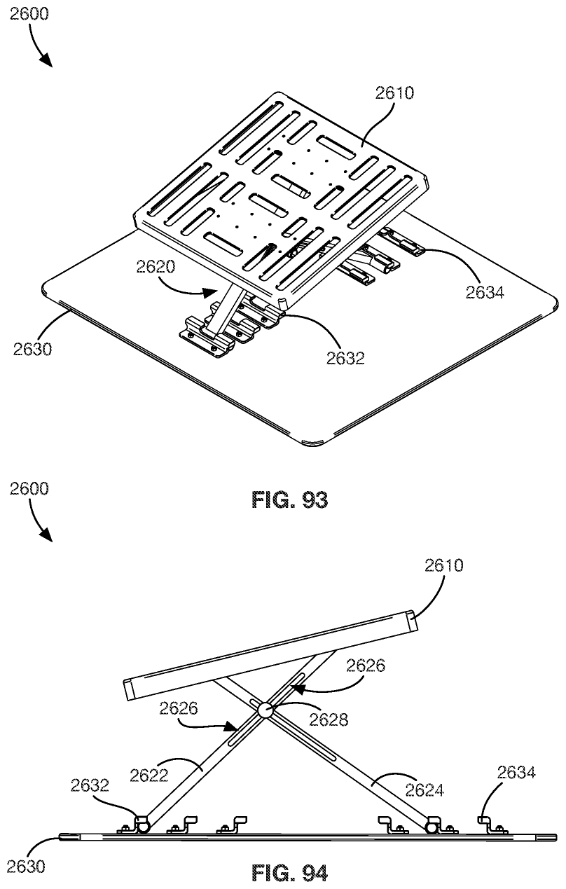

As can be seen in FIG. 92, in some implementations, when the first and second support struts 2622, 2624 are each attached to lower brackets 2632, 2634 that are equidistant from the location of the pivot connection 2628, the platform 2610 is flat—i.e., substantially parallel to the base plate 2630. The height of the platform 2610 is highest when the first and second support struts 2622, 2624 are each attached to the inner most lower bracket 2632, 2634, as is shown in FIG. 92, and can be reduced by moving the first and second support struts 2622, 2624 to brackets that are more spaced apart, such as the second or third lower brackets 2632, 2634. The platform 2610 can be tilted or pivoted away from a level or flat orientation as the first and second support struts 2622, 2624 are attached to lower brackets 2632, 2634 that are unequally spaced apart from the location of the pivot connection 2628. For example, as can be seen in FIGS. 93-94, when the first support strut 2622 is attached to the outermost first lower bracket 2632 and the second support strut 2624 is attached to the middle second lower bracket 2634 the platform 2610 is slanted in one direction.

In some implementations, the rail system (not shown) can be configured to be entirely or partially retained by features of the platform 2610 of the stabilizing base 2600 so that when the stabilizing base 2600 is covered by a sterile barrier (not shown), such as a drape, the rail system can still be attached to the stabilizing base 2600. The illustrated barriers 112 can be placed between the stabilizing base 2600 and the rail system. The rail system can be attached to the stabilizing base 2600 without attachment means, such as fasteners, that would pierce or puncture the sterile barrier arranged between the platform 2610 and the rail system. In some implementations, the rail system can be attached to the stabilizing base 2600 using magnetic plates or snap connections that do not puncture a sterile barrier. In some implementations, the horizontal position of the rail system can be adjusted relative to the platform 2610, such as by sliding the rail system forwards and backwards in relation to the platform 2610.

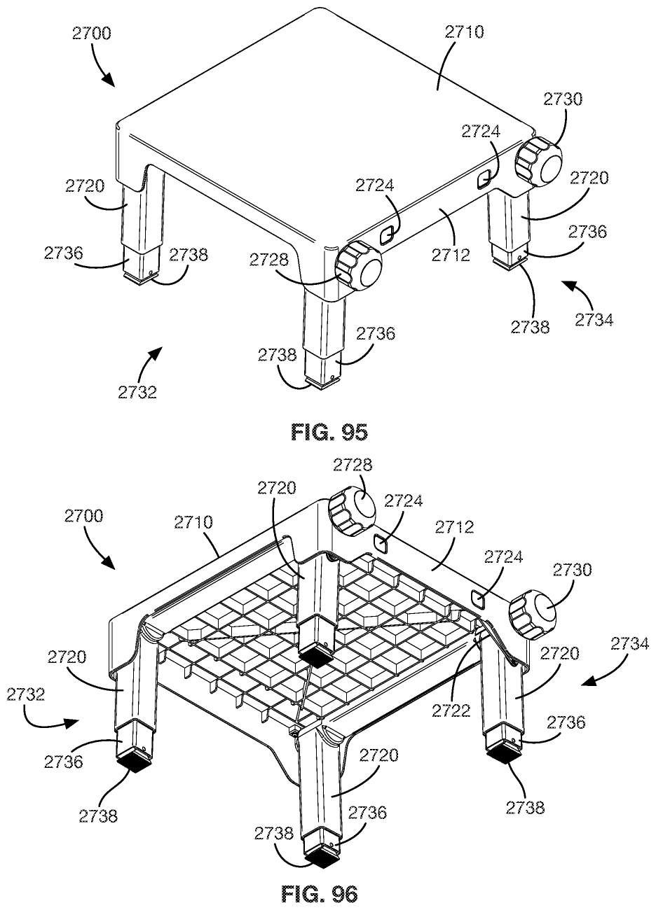

Referring now to FIGS. 95-104 an example stabilizing base 2700 for supporting a medical device/system is shown. The stabilizing base 2700 can incorporate any of the features of stabilizing bases disclosed herein and can be made from any suitable material, such as metal or plastic. The stabilizing base 2700 includes a platform 2710 that is hingeably attached to extendable support legs 2720. The support legs 2720 extend downward from the platform 2710 to an operating table (not shown) to elevate the platform 2710 above a patient laying on the operating table. In some implementations, a rail system (not shown) attaches to the platform 2710 (or, in some implementations, is integrally formed on or as part of the platform) and can receive clamps (not shown) for securing a medical device or system, such as the delivery system or catheter assembly 114, to the rail system. The rail system can be attached to the platform 2710 in any suitable way, such as, for example, with fasteners, threaded fasteners, snaps, clamps, latches, friction fit, spring-loaded clamps, hook and loop fasteners, magnets, or the like. In some implementations, the rail system and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties, which are incorporated herein by reference in their entireties. In the illustrated implementation, the platform 2710 has a rectangular shape and the support legs 2720 are arranged at the corners of the platform 2710 to spread out the points of support for the platform 2710, thereby providing stability to the stabilizing base 2700. The spacing apart of the support legs 2720 allows the stabilizing base 2700 to straddle the right or left leg of the patient so that the rail and delivery systems can be aligned with the right or left inner thigh and femoral artery of the patient.

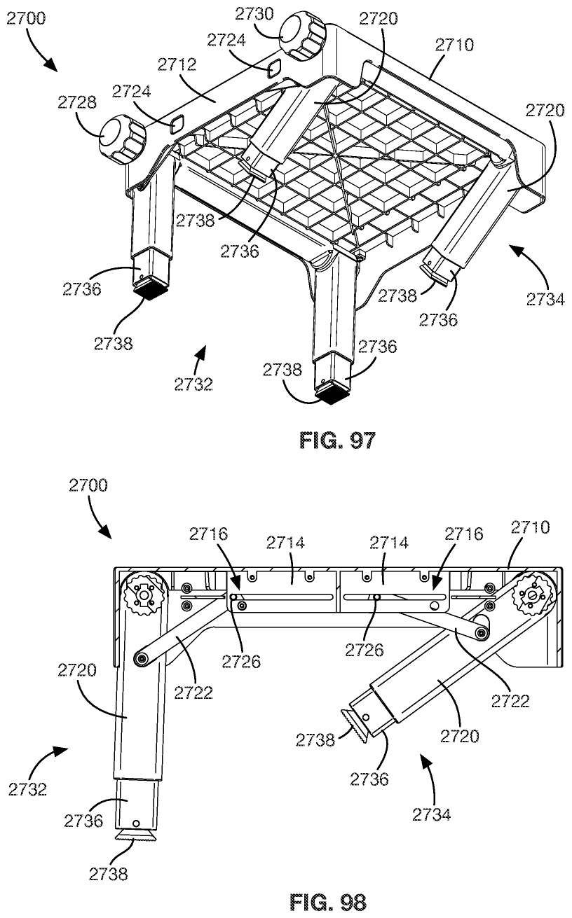

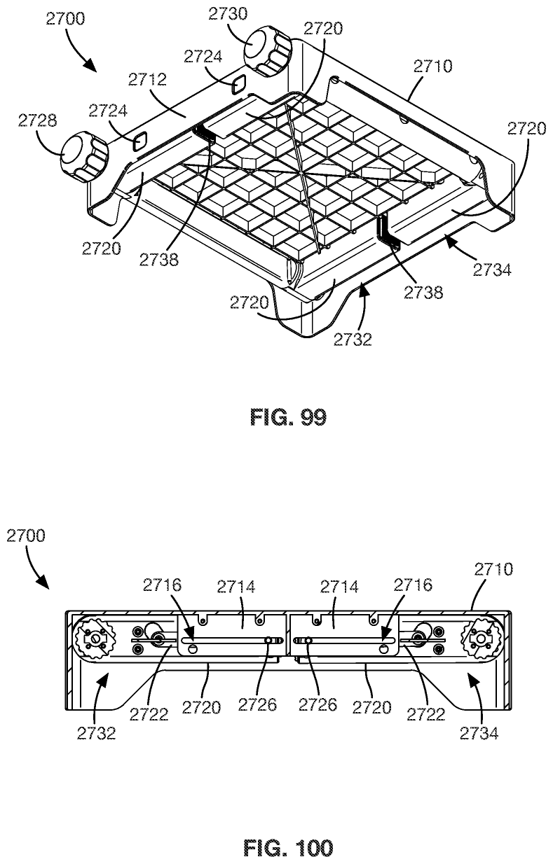

In some implementations, the support legs 2720 are each able to pivot relative to the platform 2710 between deployed (e.g., FIGS. 95-96) and stowed positions (FIGS. 99-100). The support legs 2720 are locked in the deployed position by struts 2722 and are released from the locked deployed position by actuation of a release button 2724 associated with a pair of the support legs 2720. In the stowed position the support legs 2720 are folded against the platform 2710 that can include an optional rim 2712 that extends at or beyond the support legs 2720 to conceal the support legs 2720 in the stowed position. As can be seen in FIG. 99, the support legs 2720 in the folded or stowed position are aligned end-to-end. Alternatively, support legs 2720 can be offset from one another so that the support legs 2720 attached to opposite sides of the platform 2710 overlap when folded into the stowed or folded position. Referring now to FIGS. 97-98, pressing one of the release buttons 2724 disengages a slidable end 2726 of one of the struts 2722 from a mounting plate 2714 of the platform 2710 so that the slidable end 2726 can slide laterally within a groove 2716 of the mounting plate 2714 (see FIG. 98) so that the pair of support legs 2720 that the strut 2722 is attached to can be folded upward toward the platform 2710 and into the stowed or folded position shown in FIGS. 99 and 100.

In some implementations, the height of the support legs 2720 can be adjusted by turning one of a first or a second height adjustment knob 2728, 2730 arranged at the side of the platform 2710. In the illustrated implementation, the first height adjustment knob 2728 is used to lengthen and shorten a first pair 2732 of support legs 2720 and the second height adjustment knob 2730 is used to lengthen and shorten a second pair 2734 of support legs. That is, turning the first height adjustment knob 2728 lengthens or shortens both support legs 2720 of the first pair 2732 of support legs 2720 and turning the second height adjustment knob 2730 lengthens or shortens both support legs 2720 of the second pair 2734 of support legs 2720. The support legs 2720 are lengthened or shortened by extending and retracting an extendable portion 2736 that terminates in a pivoting foot 2738. In other words, the extendable portions 2736 can be moved between a retracted position and an extended position by the rotation of the first or second height adjustment knobs 2728, 2730. The first and second height adjustment knobs 2728, 2730 can optionally include a locking device (not shown) for locking the position of the first or second height adjustment knobs 2728, 2730 to prohibit unintentional adjustment of the height of the support legs 2720.

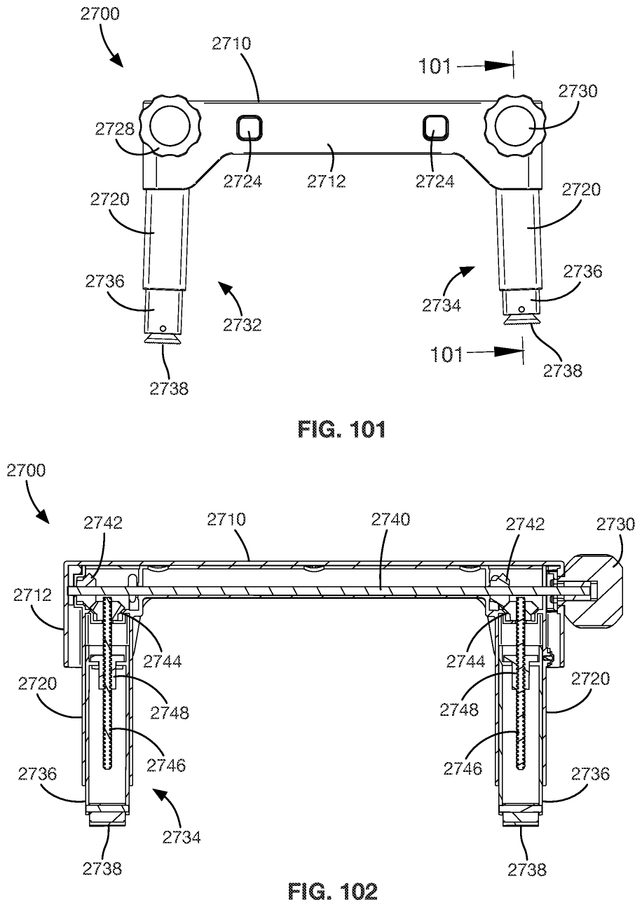

Referring now to FIG. 102, in some implementations, each of the first and second height adjustment knobs 2728, 2730 is connected to an actuation shaft 2740 that extends through both support legs 2720 of the first or second pairs 2732, 2734 of support legs 2720 that correspond to the first or second height adjustment knobs 2728, 2730. The actuation shafts 2740 extend through driving gears 2742 arranged in the platform 2710 that are engaged with driven gears 2744 that are arranged at the upper end of each support leg 2720. The driven gears 2744 are connected to threaded extension shafts 2746 that extend axially through each support leg 2720. An extension nut 2748 is threaded onto the threaded extension shaft 2746 and is attached to the extendable portion 2738 of the support leg 2720. The driving gears 2724 and the driven gears 2744 can be bevel gears or any other gear or mechanism for transferring the rotational motion of the actuation shafts 2740 to rotational motion of the threaded extension shafts 2746 through an about 90-degree angle between the actuation shafts 2740 and the threaded extension shafts 2746. It should be noted that the actuation shafts 2740 are aligned with the rotation or pivot axis of the support legs 2720 so that the support legs 2720 can be rotated between the deployed and stowed positions without affecting the extension of the support legs 2720.

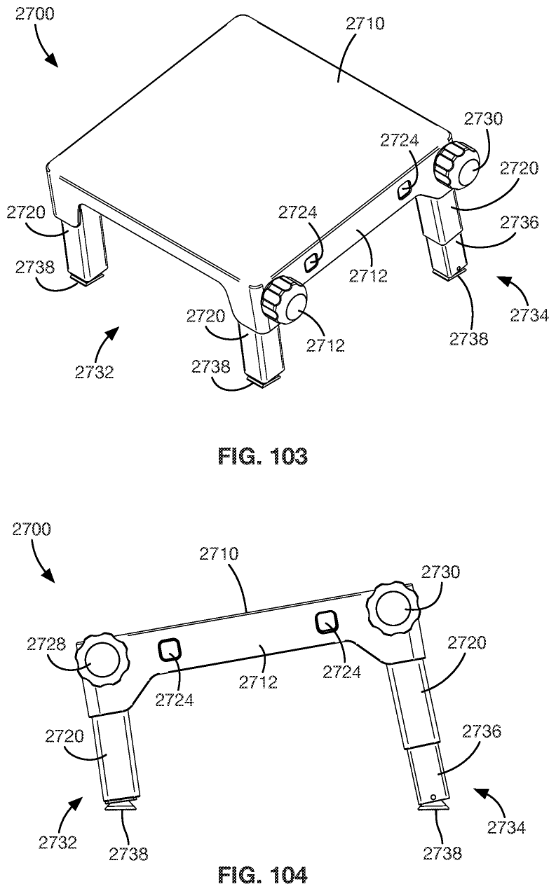

Referring now to FIGS. 103 and 104, in some implementations, the support legs 2720 in the first and second pairs 2732, 2734 can be extended and retracted to change the height and/or the tilt of the platform 2710. That is, both of the first and second pairs 2732, 2734 of support legs 2720 can be lengthened or shortened the same amount to raise or lower the platform 2710 while keeping the platform 2710 substantially parallel to the surface on which the stabilizing base 2700 is placed. The platform 2710 can be tilted by adjusting the length of the first pair 2732 of support legs 2720 to a different length than the length of the second pair 2734 of support legs 2720. As can be seen in FIG. 104, shortening the first pair 2732 of support legs 2720 causes the platform 2710 to tilt towards the shortened support legs 2720. Because the threaded extension shafts 2746 of the support legs 2720 are oriented at about a right angle to the actuation shafts 2740, forces applied to the support legs 2720 tend to not cause the driving gears 2742 or the driven gears 2744 to rotate. While the first and second height adjustment knobs 2728, 2730 can include an optional locking device (see above), the height of the support legs 2720 tends to persist until adjusted by rotation of one of the first or second height adjustment knobs 2728, 2730 because of the arrangement of the thread extension shafts 2746 and the actuation shafts 2740.

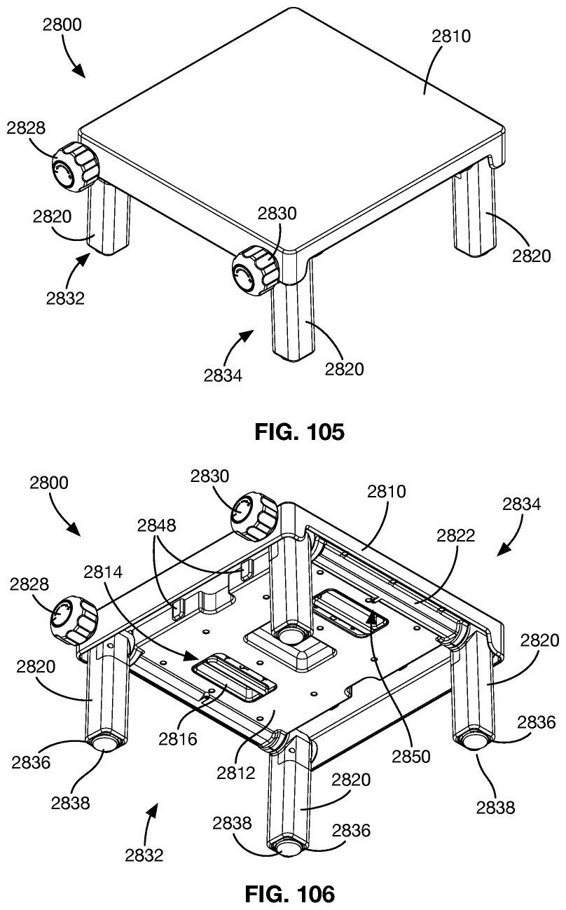

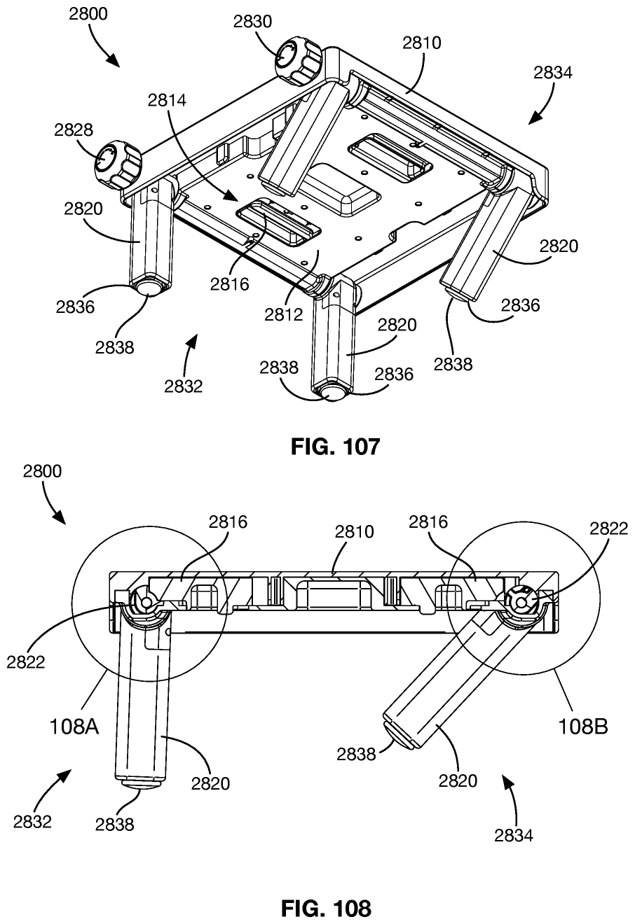

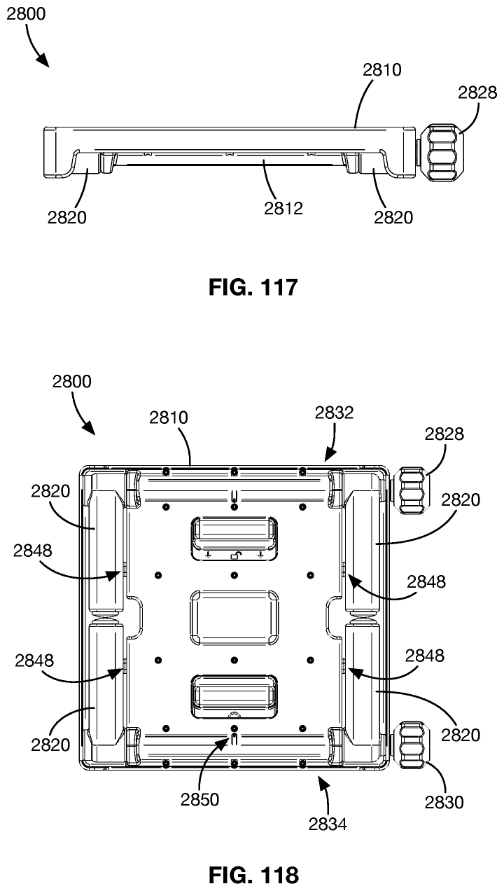

Referring now to FIGS. 105-118 an example stabilizing base 2800 for supporting a medical device/system is shown. The stabilizing base 2800 can incorporate any of the features of stabilizing bases disclosed herein and can be made from any suitable material, such as metal, plastic, etc. The stabilizing base 2800 is similar to the stabilizing base 2700 and can include any of the features of the stabilizing base 2700 described above. The stabilizing base 2800 includes a platform 2810 that is hingeably attached to extendable support legs 2820. The support legs 2820 extend downward from the platform 2810 to an operating table (not shown) to elevate the platform 2810 above a patient laying on the operating table. In some implementations, a rail system (not shown) attaches to the platform 2810 (or, in some implementations, is integrally formed on or as part of the platform) and can receive clamps (not shown) for securing a medical device or system, such as the delivery system or catheter assembly 114, to the rail system. The rail system can be attached to the platform 2810 in any suitable way, such as, for example, with fasteners, threaded fasteners, snaps, clamps, latches, friction fit, spring-loaded clamps, hook and loop fasteners, magnets, or the like. In some implementations, the rail system and/or the clamps are the same as those disclosed by U.S. Provisional Patent Application Ser. No. 63/073,392, filed on Sep. 1, 2020 and/or PCT Application No. PCT/US2021/048333, filed on Aug. 31, 2021, which are incorporated herein by reference in their entireties. In the illustrated implementation, the platform 2810 has a rectangular shape and the support legs 2820 are arranged at the corners of the platform 2810 to spread out the points of support for the platform 2810, thereby providing stability to the stabilizing base 2800. The spacing apart of the support legs 2820 allows the stabilizing base 2800 to straddle the right or left leg of the patient so that the rail and delivery systems can be aligned with the right or left inner thigh and femoral artery of the patient.

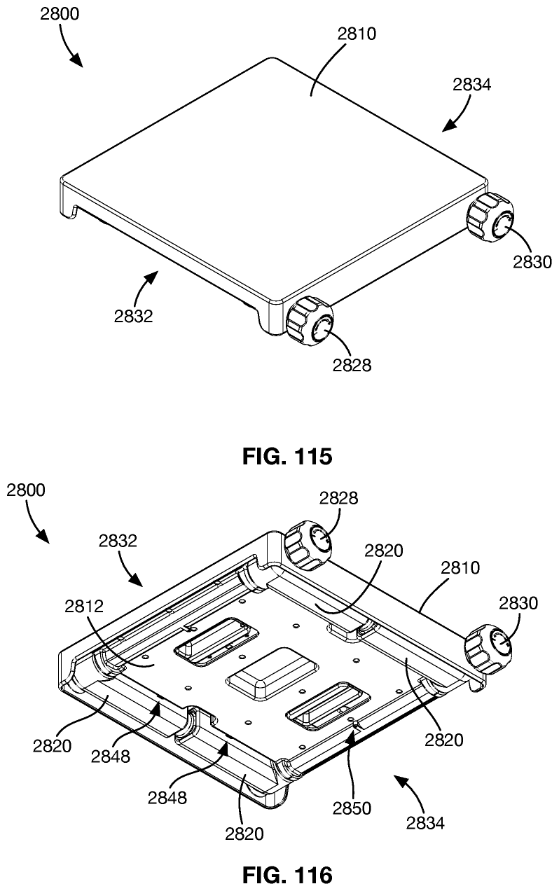

In some implementations, the support legs 2820 are each able to pivot relative to the platform 2810 between deployed (e.g., FIGS. 105-106) and stowed positions (e.g., FIGS. 115-118). A bottom cover 2812 attached to the underside of the platform 2810 includes openings 2814 that provide access to sliding latch members 2816 that can be actuated to allow the support legs 2820 to be pivoted. The bottom cover 2812 can be formed from any suitable material-such as, for example, molded or additively manufactured plastic, cast or machined metal, or the like and can be formed in one piece, multiple pieces, and/or multiple layers of varying materials. A retaining member 2848 (FIGS. 106, 109, 116, and 118) is provided between the bottom cover 2812 and each support leg 2820 to retain the support legs 2820 in the stowed position. The retaining member or pad 2848 is sized to form a tight or interference fit between the bottom cover 2812 and the support leg 2820 so that friction between the retaining member 2848 and the support leg 2820 prohibits unwanted rotation of the support leg 2820. The retaining member 2848 can be attached to the bottom cover 2812 in a wide variety of ways, such as, for example, via insertion into a slot in the bottom cover 2812, adhesive, one or more fasteners, induction welding, or the like. The retaining member 2848 can also be formed by one or more protrusions from the bottom cover 2812 that can be the same material as the bottom cover 2812 or can be coated with an elastomeric coating to provide additional friction with the support leg 2820. The retaining member 2848 can take on a wide variety of forms, such as, a strap, a latch, a ball detent, or the like.

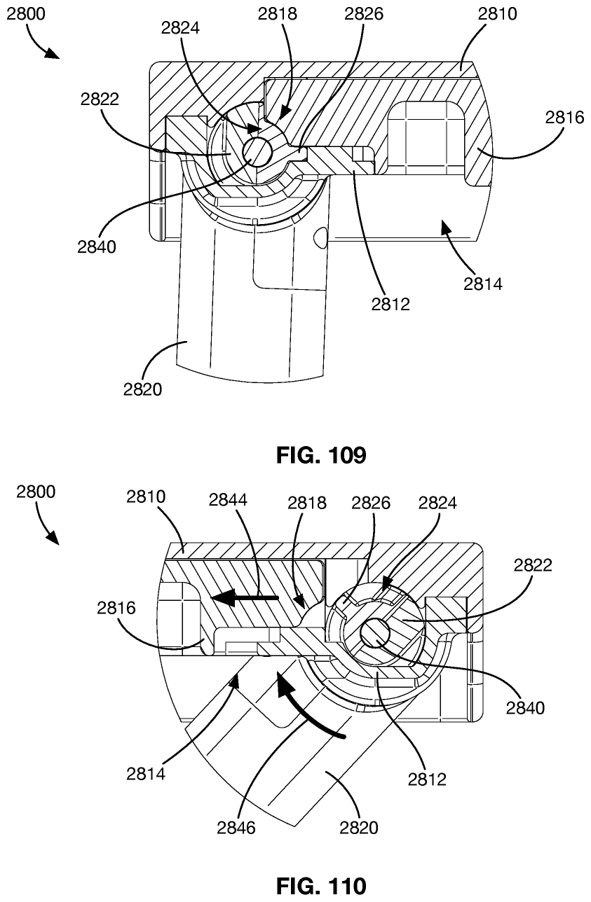

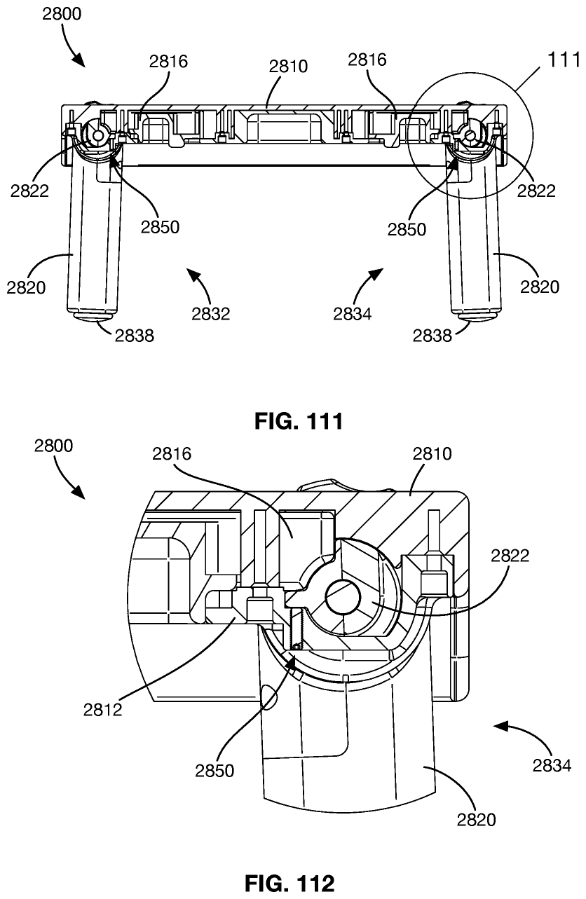

Referring now to FIG. 109 (showing an enlarged detail view of area 108A of FIG. 108) and FIG. 110 (showing an enlarged detail view of area 108B of FIG. 108), the mechanism for locking and unlocking the rotation of the supporting legs 2820 is shown. A rotating catch member 2822 extends between and is connected to the support legs 2820 in each of the first and second pairs of support legs 2828, 2830. The rotating catch member 2822 includes a recess 2824 that corresponds to an engagement end 2818 of the sliding latch member 2816. An extension member 2826 of the rotating catch member 2822 is captured between the sliding latch member 2816 and the bottom cover 2812, thereby prohibiting rotation of the support legs 2820. When the sliding latch member 2816 is retracted away from the rotating catch member 2822 in a retracting direction 2844, the extension member 2826 of the rotating catch member 2822 is free from the engagement end 2818 of the sliding latch member 2816. As a result, the support legs 2820 and the rotation catch member 2822 are able to rotate in a collapsing or stowing direction 2846 from the deployed to the stowed position. Tightening screws 2850 are shown in FIGS. 111-112

Referring now to FIGS. 111-112, a cross-sectional view of the example stabilizing base 2800 is shown to reveal tightening screws 2850. The tightening screws 2850 extend through the bottom cover 2812 to engage the extension member 2826 of the rotating catch member 2822 to take up any slop or gaps between the extension member 2826, bottom cover 2812, and the sliding latch member 2816. During manufacturing of the stabilizing base 2800, the tightening screw 2850 is tightened when the support legs 2820 are in the open or deployed position to prohibit undesired rotation of the support legs 2820 that could allow the stabilizing base 2800 to wobble or move. The extension member 2826 moves away from the tightening screw 2850 as the support legs 2820 are rotated into the closed or stowed position. When the support legs 2820 are deployed again, the tightening screw 2850 stops the rotation of the extension member 2826 in the previously determined location. Thus, the tightening screw 2850 also operates as a calibration mechanism to calibrate the outermost limit of the rotation of the support legs 2820. In some implementations, portions of the bottom cover 2812 covering the rotation and extension mechanism for the support legs 2820 have an increased stiffness as compared to the rest of the bottom cover 2812 to provide additional support to, for example, the tightening screw 2850 and the rotating catch member 2822. The additional stiffness of one or more portions of the bottom cover 2812 can be provided via adding layers of a stiff material (e.g., a metal plate or sheet) to a relatively more flexible material (e.g., injection molded plastic). As another example, the additional stiffness in a desired location can be provided by forming a separate cover piece out of a die cast metal that abuts or overlaps portions of the other pieces of the bottom cover 2812.

In the deployed position, the height of the support legs 2820 can be adjusted by turning one of a first or a second height adjustment knob 2828, 2830 arranged at the side of the platform 2810. In the illustrated implementation, the first height adjustment knob 2828 is used to lengthen and shorten a first pair 2832 of support legs 2820 and the second height adjustment knob 2830 is used to lengthen and shorten a second pair 2834 of support legs. That is, turning the first height adjustment knob 2828 lengthens or shortens both support legs 2820 of the first pair 2832 of support legs 2820 and turning the second height adjustment knob 2830 lengthens or shortens both support legs 2820 of the second pair 2834 of support legs 2820. The support legs 2820 are lengthened or shortened by extending and retracting an extendable portion 2836 that terminates in a rounded foot 2838. In other words, the extendable portions 2836 can be moved between a retracted position and an extended position by the rotation of the first or second height adjustment knobs 2828, 2830. The first and second height adjustment knobs 2828, 2830 can optionally include a locking device (not shown) for locking the position of the first or second height adjustment knobs 2828, 2830 to prohibit unintentional adjustment of the height of the support legs 2820. The mechanism that operates to extend and retract the support legs 2820 is similar to the actuation shaft 2740 and gears 2742, 2744 of the stabilizing base 2700 that is shown in FIG. 102.

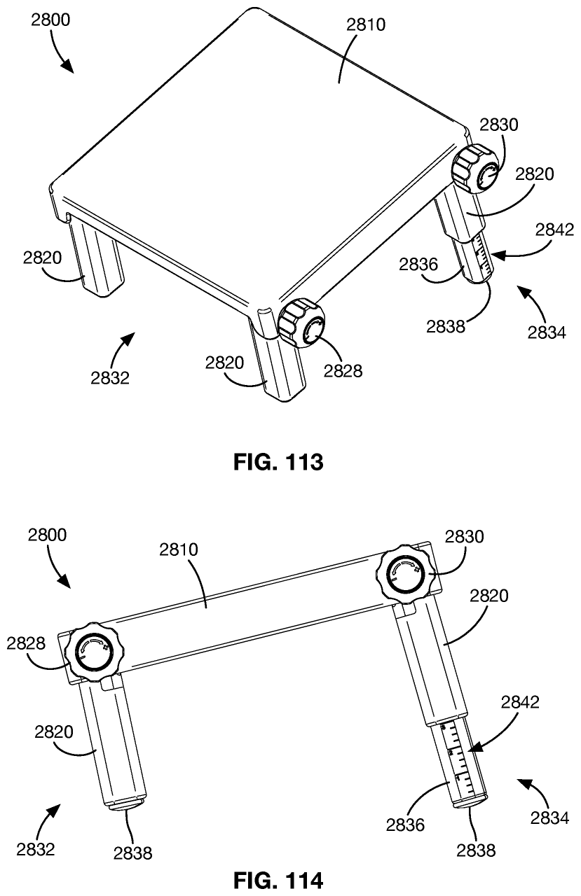

Referring now to FIGS. 113 and 114, in some implementations, the support legs 2820 in the first and second pairs 2832, 2834 can be extended and retracted to change the height and/or the tilt of the platform 2810. That is, both of the first and second pairs 2832, 2834 of support legs 2820 can be lengthened or shortened the same amount to raise or lower the platform 2810 while keeping the platform 2810 substantially parallel to the surface on which the stabilizing base 2800 is placed. The extendable portions 2836 of the support legs 2820 can optionally include a graduated scale 2842 (e.g., a ruler as shown in FIGS. 113-114) to help the user measure the amount of extension of each pair of support legs 2832, 2834 to more easily reproduce a desired tilt angle or height of the platform 2810. The platform 2810 can be tilted by adjusting the length of the first pair 2832 of support legs 2820 to a different length than the length of the second pair 2834 of support legs 2820. As can be seen in FIG. 114, shortening the first pair 2832 of support legs 2820 causes the platform 2810 to tilt towards the shortened support legs 2820. While the first and second height adjustment knobs 2828, 2830 can include an optional locking device (see above), the height of the support legs 2820 tends to persist until adjusted by rotation of one of the first or second height adjustment knobs 2828, 2830 because of the arrangement of the thread extension shafts (not shown) and the actuation shafts 2840 (see FIG. 109) used to extend and retract the support legs 2820.

While various inventive aspects, concepts and features of the disclosures can be described and illustrated herein as embodied in combination in the examples shown and described, these various aspects, concepts, and features can be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures-such as alternative materials, structures, configurations, methods, devices, and components, alternatives as to form, fit, and function, and so on—can be described herein, such descriptions are not intended to be a complete or exhaustive list of available alternative implementations, whether presently known or later developed. Those skilled in the art can readily adopt one or more of the inventive aspects, concepts, or features into additional implementations and uses within the scope of the present application even if such implementations are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the treatment techniques, methods, operations, steps, etc. described or suggested herein can be performed on a living animal or on a non-living simulation, such as on a cadaver, cadaver heart, simulator (e.g., with the body parts, tissue, etc. being simulated), etc. The words used in the claims have their full ordinary meanings and are not limited in any way by the description of the implementations in the specification.

I claim:

1. A system for supporting a medical device, the system comprising:
a stabilizing base comprising:
a plurality of legs;
a platform attached to the plurality of legs;
a stabilizing system for receiving the medical device, wherein the stabilizing system is removably attachable to the platform; and
wherein one or more of the plurality of legs is adjustable to change one or more of a vertical, horizontal, and angular position of the platform.

2. The system of claim 1, wherein one or more of the plurality of legs comprise an outer portion and an inner portion movably disposed within said outer portion.

3. The system of claim 1, further comprising a first plate and a second plate, wherein the first plate and the second plate are parallel to one another and extend vertically downward from the platform on either side of the platform.

4. The system of claim 3, further comprising a knob mechanism that extends through at least one of the first plate and the second plate.

5. The system of claim 1, further comprising a mechanism to control the height of one or more of the plurality of legs.

6. The system of claim 1, wherein the stabilizing system is a rail system for receiving the medical device, wherein the rail system is removably attachable to the platform.

7. The system of claim 1, wherein the medical device is a delivery system couplable to the stabilizing system, and the system includes the delivery system.

8. The system of claim 1, wherein the plurality of legs comprises a first pair of legs and a second pair of legs.

9. The system of claim 8, wherein the first pair of legs and the second pair of legs are foldable between a stowed position and a deployed position.

10. The system of claim 9, further comprising a first strut for retaining the first pair of legs in the deployed position and a second strut for retaining the second pair of legs in the deployed position, wherein the first strut and the second strut are slideably attached to the platform and can be released to facilitate folding of the first and second pairs of legs from the deployed to the stowed position.

11. The system of claim 8, further comprising a first height adjustment knob for adjusting the height of the legs of the first pair of legs and a second height adjustment knob for adjusting the height of the legs of the second pair of legs.

12. The system of claim 8 further comprising:
a first height adjustment knob, a second height adjustment knob, an actuation shaft, a pair of drive gears, a pair of driven gears, and a pair of extension shafts;
extendable portions of each support leg are attached to the extension shafts;
wherein the drive gears are fixedly attached to the actuation shaft and the driven gears are fixedly attached to the extension shafts; and
wherein rotating one of the first height adjustment knob and the second height adjustment knob rotates both of the extension shafts via the drive gears and driven gears to increase and decrease the height of the legs.

13. The system of claim 8, wherein the platform can be tilted by adjusting the height of the first pair of support legs to a lower height than the height of the second pair of legs.

14. The system of claim 9, further comprising a retaining member for retaining each of the plurality of legs in the stowed position.

15. The system of claim 14, wherein the retaining member is attached to a bottom cover of the platform.

16. A system for supporting a medical device, the system comprising:
a stabilizing base comprising:
a first pair of legs;
a second pair of legs;
a platform attached to the first pair of legs and the second pair of legs;
wherein each leg of the first pair of legs and the second pair of legs comprises an outer portion and an inner portion movably disposed within said outer portion;
a first knob coupled to the first pair of legs configured to adjust the height of the first pair of legs;
a second knob coupled to the second pair of legs configured to adjust the height of the second pair of legs;
wherein the first pair of legs are pivotable between a stowed position and a deployed position;
wherein the second pair of legs are pivotable between a stowed position and a deployed position;
a stabilizing system for receiving the medical device, wherein the stabilizing system is removably attachable to the platform; and
wherein the height of the first pair of legs and the height of the second pair of legs are each in independently adjustable to change a vertical position of the platform, a horizontal position of the platform, and angular position of the platform.

17. The system of claim 16, further comprising a first strut for retaining the first pair of legs in the deployed position and a second strut for retaining the second pair of legs in the deployed position, wherein the first strut and the second strut are slideably attached to the platform and can be released to facilitate folding of the first and second pairs of legs from the deployed to the stowed position.

18. The system of claim 16, wherein each of the first pair of legs and the second pair of legs comprise:
an actuation shaft, a pair of drive gears, a pair of driven gears, and a pair of extension shafts;
extendable portions of each support leg are attached to the extension shafts;
wherein the drive gears are fixedly attached to the actuation shaft and the driven gears are fixedly attached to the extension shafts; and
wherein rotating one of the first knob and the second knob rotates both of the extension shafts via the drive gears and driven gears to increase and decrease the height of the legs.

19. The system of claim 16, wherein the platform can be tilted by adjusting the height of the first pair of support legs to a lower height than the height of the second pair of legs.

20. A system for supporting a medical device, the system comprising:
a stabilizing base comprising:
an arm;
a mount configured to attach the arm to an operating table; and
a stabilizing system for receiving the medical device, wherein the stabilizing system is removably attached to the arm, and wherein the vertical, horizontal, and radial position of the stabilizing system is adjustable relative to the mount.

21. A system comprising:
a stabilizing mat comprising:
a first ridge and a second ridge;
a channel between the first ridge and the second ridge; and
a catheter assembly positionable in the channel.
